(19) United States
(12) Patent Application Publication
(10) Pub. No.: US 2025/0027130 A1
(43) Pub. Date: Jan. 23, 2025

(54) Nucleic Acid Synthesis and Sequencing Using Tethered Nucleoside Triphosphates

(71) Applicant: The Regents of the University of California, Oakland, CA (US)

(72) Inventors: Alanna Schepartz Shrader, Berkeley, CA (US); Riley Carson B. Fricke, Berkeley, CA (US); Cameron V. Swenson, Berkeley, CA (US)

(73) Assignee: The Regents of the University of California, Oakland, CA (US)

(21) Appl. No.: 18/804,140

(22) Filed: Aug. 14, 2024

Related U.S. Application Data
(63) Continuation of application No. PCT/US23/63304, filed on Feb. 26, 2023.
(60) Provisional application No. 63/314,406, filed on Feb. 27, 2022.

(51) Int. Cl. C12P 19/34; C12N 9/00

(52) U.S. Cl. CPC C12P 19/34; C12N 9/93; C12Y 601/01026

(57) ABSTRACT

Methods to generate novel acyl-tRNA species deploy an orthogonal synthetase that accepts α-hydroxy acids, α-thio acids, N-formyl-L-α-amino acids, and/or α-carboxyl acid monomers (malonic acids) that are formally precursors to polyketide natural products.

### CROSS-REFERENCES TO RELATED APPLICATIONS

This application is a continuation of PCT/US23/63304, filed Feb. 26, 2023, which claims priority to U.S. Provisional Application No. 63/314,406, filed Feb. 27, 2022, the disclosures of which are hereby incorporated by reference in its entirety for all purposes.

### GOVERNMENT SUPPORT CLAUSE

This invention was made with government support under National Science Foundation award 2002182. The government has certain rights in the invention.

### REFERENCE TO A SEQUENCE LISTING

A Sequence Listing in XML format is incorporated by reference into the specification. The name of the XML file containing the Sequence Listing is B22-095-2US.xml. The XML file is 44,024 bytes and was created on Sep. 30, 2024.

### INTRODUCTION

All extant organisms biosynthesize polypeptides in an mRNA template-dependent manner using a translation apparatus composed of ribosomes, aminoacyl-tRNA synthetases, tRNAs, and a host of ancillary factors. Repurposing this translation apparatus for the templated synthesis of mixed-sequence hetero-oligomers—especially those containing non-L-α-amino acids—would provide a biological route to sequence-defined non-peptide polymers with novel, tunable, evolvable properties and protein therapeutics with improved stability and expanded recognition potential. Chemical methods support the synthesis of sequence-defined non-peptide polymers^{1,2} but are limited to small scale and produce considerable waste. Polymerization methods support the synthesis of sequence-controlled materials, but without rigorous sequence-definition. By contrast, cellular methods based on the translation apparatus generate little waste, especially on a large scale, achieve greater chain lengths, and are scalable for industrial production.

It has been established over the past decade that many non-L-α-amino acids, including α-hydroxy acids^{3,4}, D-α-⁵, linear⁶⁻⁸ and cyclic⁹ β-, γ-^{10,11}, and long chain amino acids¹², α-aminoxy and α-hydrazino acids¹³, α-thio acids¹⁴, aramids^{15,16}—even 1,3-dicarbonyl monomers that resemble polyketide precursors¹⁶—are accepted by E. coli ribosomes in small-scale in vitro reactions. The structural and electronic diversity of these monomers reiterates the important role of proximity in promoting bond-forming reactions within the E. coli peptidyl transferase center (PTC)¹⁷. However, there are scant examples in which non-L-α-amino acids have been incorporated into proteins in vivo¹⁸⁻²³. The absence of orthogonal aminoacyl-tRNA synthetase (aaRS) enzymes that accept non-L-α-amino acid substrates is the primary bottleneck limiting the production of sequence-defined, non-peptide hetero-polymers in vivo using wild-type or engineered ribosomes.

In E. coli, two families of orthogonal aaRS enzymes have been employed widely to introduce hundreds of different non-canonical α-amino acid monomers²⁴⁻²⁸ into protein. The first includes pyrrolysyl-tRNA synthetase (PylRS) enzymes from methanogenic archaea and bacteria whose natural substrate is pyrrolysine²⁹, an L-α-amino acid found in the active sites of certain enzymes involved in methane metabolism³⁰. The second includes a large family of enzymes derived from Methanocaldococcus jannaschii tyrosyl-tRNA synthetase (MjTyrRS)^{24,31}. These two enzyme classes differ in how they recognize the α-amine of a bound substrate. While MjTyrRS recognizes the substrate α-amine via multiple, direct hydrogen bonds to the side chains of Q173, Q176, and Y151 (PDB: 1J1U)³², Methanosarcina mazei PylRS (MmPylRS) instead uses water-mediated hydrogen bonds to the N346 side chain and L301 and A302 backbone amides (PDB: 2ZCE; FIG. 1A)³³. These differences were exploited by Kobayashi et al. to acylate the cognate tRNA Mm-tRNA^{Pyl} with a series of conservative N^{ε}-Boc-L-lysine (L-BocK) analogs containing —OH, —H and —NHCH₃ in place of the α-amine (FIG. 1B)²⁰.

### SUMMARY OF THE INVENTION

The absence of orthogonal aminoacyl-tRNA synthetases that accept non-L-α-amino acids is the primary bottleneck hindering the in vivo translation of sequence-defined hetero-oligomers. Here we disclose PylRS enzymes that accept α-hydroxy acids, α-thio acids, N-formyl-L-α-amino acids, and α-carboxyl acid monomers (malonic acids) that are formally precursors to polyketide natural products.

The invention provides methods and compositions for generating novel acyl-tRNA species, including orthogonal synthetases for polyketide precursors.

In an aspect the invention provides a method to generate novel acyl-tRNA species, comprising deploying an orthogonal synthetase that accepts α-hydroxy acids, α-thio acids, N-formyl-L-α-amino acids, and/or α-carboxyl acid monomers (malonic acids) that are formally precursors to polyketide natural products.

In an aspect, the invention provides a composition or kit comprising an isolated orthogonal synthetase that accepts α-hydroxy acids, α-thio acids, N-formyl-L-α-amino acids, and/or α-carboxyl acid monomers (malonic acids) that are formally precursors to polyketide natural products.

In embodiments:
the orthogonal synthetase accepts α-hydroxy acids, α-thio acids, N-formyl-L-α-amino acids, and α-carboxyl acid monomers (malonic acids) that are formally precursors to polyketide natural products;
the orthogonal synthetase is a pyrrolysyl-tRNA synthetase (PylRS);
the orthogonal synthetase is a pyrrolysyl-tRNA synthetase (PylRS), and the PylRS is a Methanomethylophilus alvus PylRS (MaPylRS) or a MaPylRS substitution variant;
the orthogonal synthetase is a pyrrolysyl-tRNA synthetase (PylRS), and the PylRS is a Methanomethylophilus alvus PylRS (MaPylRS) substitution variant comprising substitutions at N166 and V168;
the orthogonal synthetase is a pyrrolysyl-tRNA synthetase (PylRS), and the PylRS is a Methanomethylophilus alvus PylRS (MaPylRS) substitution variant comprising MaFRS1 (N166A, V168L), MaFRS2 (N166A, V168K), or MaFRSA (N166A, V168A);
the method further comprising providing the acyl-tRNA species in a translation system, wherein the non-L-α-amino acid is incorporated into a protein; or
the method further comprising providing the acyl-tRNA species in a translation system, wherein the non-L-α-amino acid is incorporated into a sequence-defined non-protein hetero-polymer.

The invention encompasses all combinations of the particular embodiments recited herein, as if each combination had been laboriously recited.

### BRIEF DESCRIPTION OF THE DRAWINGS

FIGS. 1A-1E. Promiscuous activity of wild-type MaPylRS. FIG. 1A, The α-amines of L-α-amino acids are recognized differently by M. mazei PylRS (MmPylRS, left)³⁴ and M. jannaschii TyrRS (MjTyrRS, right)³³. FIG. 1B, N^{ε}-Boc-L-lysine (L-BocK, 1) analogs evaluated as substrates for MaPylRS. FIG. 1C, Ribonuclease A (RNAse A) assay used to detect acylation of Ma-tRNA^{Pyl} with BocK analogs shown in FIG. 1B. FIG. 1D, LC-HRMS analysis of Ma-tRNA^{Pyl} acylation reactions after RNAse A digestion. Peak masses correspond to adenosine nucleoside 6 acylated on the 2′ or 3′ hydroxyl with the indicated monomer. Two isobaric peaks are observed because the sample consists of adenosine that is acylated on either the 2′ or the 3′ hydroxyl group. Although there is evidence that PylRS from Methanosarcina barkeri and Desulfitobacterium hafniense add Pyl to only the 3′-hydroxyl group⁴⁵, isomerization between 2′ and 3′-isomers occurs rapidly⁴¹ and we are not able to identify which peak corresponds to which isomer. FIG. 1E, Heat map illustrating the relative activities of substrates 1-5 for MaPylRS as determined by intact tRNA analysis as described in Methods. Reported yields are percentages based on intact tRNA analysis. Percent yields are calculated as the ratio of tRNA acylated with the monomer of interest divided by the total tRNA detected by the LC-MS analysis. Black indicates no reaction product detected; X indicates the substrate was not investigated.

FIGS. 2A-2D. MaFRS1 and MaFRS2 process phenylalanine analogs with substitutions at the α-amine. FIG. 2A, Phenylalanine analogs evaluated as substrates for MaFRS1 and MaFRS2. FIG. 2B, Adenosine nucleoside formed during RNAse A digestion of acyl-tRNA. FIG. 2C, LC-HRMS analysis of Ma-tRNA^{Pyl} acylation reactions after RNAse A digestion. Adenine nucleoside 12 acylated on the 2′- or 3′-hydroxyl of the 3′ terminal ribose of Ma-tRNA^{Pyl} could be detected in MaFRS1 and MaFRS2 reactions with L-Phe 7 and substrate 8; substrates 9 and 10 (Z=—H, —NHCH₃) showed more modest reactivity. FIG. 2D, Heat map illustrating the relative yields with substrates 7-11 for MaFRS1 and MaFRS2 as determined by intact tRNA analysis as described in Methods. Reported yields are percentages based on intact tRNA analysis. Black indicates no reaction product detected.

FIGS. 3A-3D. MaFRS1 and MaFRS2 process substrates bearing novel α-substituents. FIG. 3A, LC-HRMS analysis of Ma-tRNA^{Pyl} acylation reactions using MaFRS1 or MaFRS2 following RNAse A digestion. Adenosine nucleoside 12 acylated on the 2′- or 3′-hydroxyl of the 3′ terminal ribose of Ma-tRNA^{Pyl} could be detected in MaFRS1 and MaFRS2 reactions with α-thio acid 13, α-carboxyl acid 14, and N-formyl-L-Phe 15. FIG. 3B, LC-MS analysis of intact tRNA products confirms that monomers 13-15 are substrates for MaFRS1 and MaFRS2. Reported yields are percentages based on intact tRNA analysis. We note that intact tRNAs acylated with 2-benzylmalonate 14 showed evidence of decarboxylation (indicated by a D). No evidence for decarboxylation was observed when the same acyl-tRNAs were evaluated using the RNAse A assay, suggesting that decarboxylation occurs either during workup or during the LC-MS run. FIG. 3C, Heat map illustrating the relative activities of substrates 13-15 with MaFRS1 and MaFRS2 as determined by intact tRNA analysis as described in Methods. Black indicates no reaction product detected. Initially no acyl-tRNA was detected when MaFRS1 was incubated with 13, but when the enzyme concentration was increased 5-fold, acyl-tRNA was detected with mono- and diacyl yields of 0.7 and 9.7%, respectively. FIG. 3D, Turnover of MaFRS1 over time with L-Phe 7 and 2-benzylmalonate 14 using the malachite green assay. Data from three replicates are shown.

FIGS. 4A-4D. MaFRSA selectively acylates Ma-tRNA^{Pyl} with meta-substituted 2-benzylmalonate derivatives. FIG. 4A, LC-HRMS analysis of Ma-tRNA^{Pyl} acylation products after digestion with RNAse A. FIG. 4B, LC-MS analysis of intact tRNA products confirms that meta-substituted 2-benzylmalonates 17-19 are substrates for MaFRSA. We note that intact tRNAs acylated with meta-substituted 2-benzylmalonates 17-19 showed evidence of decarboxylation (indicated by a D). No evidence for decarboxylation was observed when the same acyl-tRNAs were evaluated using the RNAse A assay, suggesting that decarboxylation occurs either during workup or during the LC-MS run. FIG. 4C, Heat map illustrating the relative activities of L-Phe 7 and substrates 17-19 with MaFRS1, MaFRS2, and MaFRSA. Black indicates no reaction product detected. FIG. 4D, Turnover of MaFRSA over time with meta-CF₃-L-Phe and meta-CF₃-2-BMA 18 using the malachite green assay. Data from three replicates are shown.

FIGS. 5A-5F. Structure of MaFRSA bound to meta-CF₃-2-BMA and AMP-PNP reveals basis for distinct reactivity at pro-R and pro-S substrate carboxylates. FIG. 5A, MaFRSA dimer containing two non-identical chains in the asymmetric unit. FIG. 5B, Alignment of the active sites of chains A (light purple) and B (dark purple) reveals meta-CF₃-2-BMA (grays) bound in two alternate conformations. FIG. 5C, In chain A, meta-CF₃-2-BMA is coordinated by an extensive hydrogen bond network (orange dashes) that positions the pro-R carboxylate oxygen for nucleophilic attack (blue dashes); interatomic distances are shown over dashed lines in Å. FIG. 5D, In chain B, meta-CF₃-2-BMA is coordinated by similar hydrogen bonds, but in this case the pro-S carboxylate is rotated away from AMP-PNP with a loss of the hydrogen bond to R150 (red dashes) and a longer distance between the pro-S carboxylate nucleophile and the α-phosphate of AMP-PNP. FIG. 5E, Alignment of active site A with WT MmPylRS bound to Pyl and AMP-PNP (PDB: 2ZCE, blue)³⁴ illustrates the difference between the water-mediated hydrogen bonds (yellow dashes) to the α-amine of Pyl in PylRS versus the direct carboxyl to backbone hydrogen bonding of m-CF₃-2-BMA bound to MaFRSA. FIG. 5F, Comparison of active site B with MmBtaRS (N346G/C348Q) bound to Bta (PDB: 4ZIB, red)⁵⁷ reveals similar binding modes with hydrogen bonds (yellow dashes) between the substrate carboxylate and amide backbone of the enzyme when N166/N346 (Ma/Mm numbering) is mutated.

FIGS. 6A-6E. In vitro and in vivo incorporation of novel monomers. FIG. 6A, Workflow for in vitro translation via codon skipping of the peptide VDYKDDDDK (SEQ ID NO:1). FIG. 6B, Extracted ion chromatograms (EICs) and mass spectra of peptide products obtained using Ma-tRNA^{Pyl}-ACC charged with monomers 7, 13-15 by MaFRS1 (7 and 15) or MaFRS2 (13 and 14). Insets show mass spectra for major ions used to generate the EIC of the translated peptide initiated with the indicated monomer. Expected (exp) and observed (obs) m/z peaks in mass spectra are as follows: L-Phe 7 (M+3H), exp: 420.51906, obs: 420.52249; α-SH 13 (M+2H), exp: 638.75554, obs: 638.75614; N-fPhe 15 (M+2H), exp: 644.27242, obs: 644.27167; and 2-BMA 14 (M+2H), exp: 644.76442, obs: 644.76498. FIG. 6C, Workflow for in vivo incorporation of monomers 1, 2, 20, and 21 at position 200 of sfGFP. FIG. 6D, Intact protein mass spectra of sfGFP variants purified from DH10B cells co-expressing MaPylRS (top) or MaFRSA (bottom) in the presence of 1 mM BocK (1), α-OH BocK (2), m-trifluoromethyl phenylalanine (20), or α-OH m-trifluoromethyl phenylalanine (21). FIG. 6E, Fidelity (%) of sfGFP containing the indicated residue at position 200 when expressed in E. coli DH10B (columns i-vii) or DH10B ΔaspC ΔtyrB (columns viii-ix) using either TB (columns i-v, viii-ix) or a defined media lacking glutamate (columns vi-vii).

FIGS. 7A-7C. FIG. 7A, Structural alignment of the M. mazei PylRS (MmPylRS) catalytic domain (PDB 2ZCE) and M. alvus PylRS (MaPylRS) (PDB 6JP2). The two active site residues substituted in FRS1, FRS2, and FRSA are shown explicitly. FIG. 7B, Sequence alignment of MmPylRS (SEQ ID NO:3) and MaPylRS (SEQ ID NO:2) using the EMBOSS Needle software⁹³. FIG. 7C, Sequences of the four enzymes (SEQ ID NOS:3, 4-6) used in this study with differences highlighted in blue.

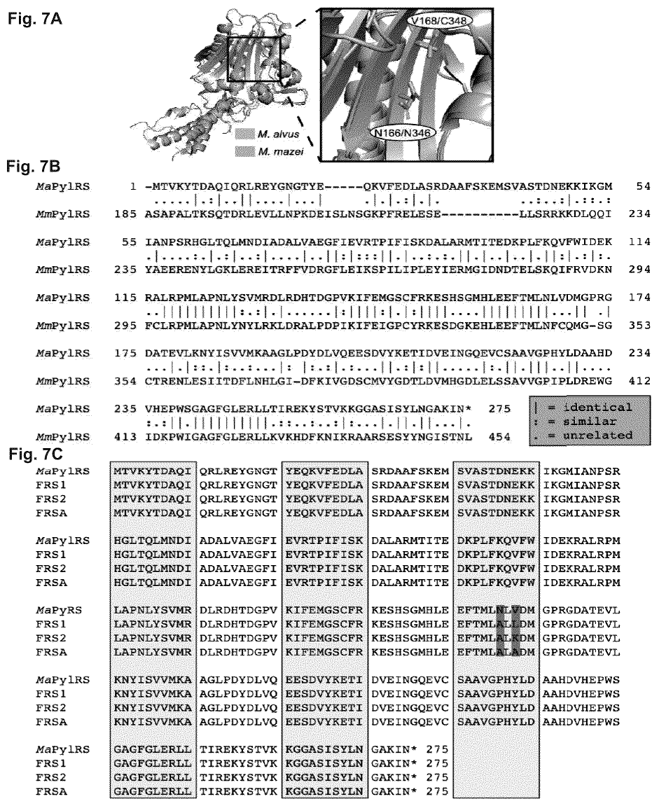

FIGS. 8A-8G. FIG. 8A, SDS-PAGE; FIG. 8B, LC-MS; and FIG. 8C, analytical FPLC chromatograms of purified MaPylRS, MaFRS1, MaFRS2, and MaFRSA used in biochemical experiments. FIG. 8D, Urea-PAGE; and FIG. 8E, LC-MS analysis of Ma-tRNA^{Pyl}. FIG. 8F, SDS-PAGE and FIG. 8G, LC-MS analysis of MaFRSA used for crystallography. We note that the MaFRSA in FIG. 8B is extended by an N-terminal His-tag and linker (GSSHHHHHHSSGLVPRGSH-) (SEQ ID NO:7), whereas the MaFRSA used for crystallography in FIG. 8G only contains an N-terminal GSH scar.

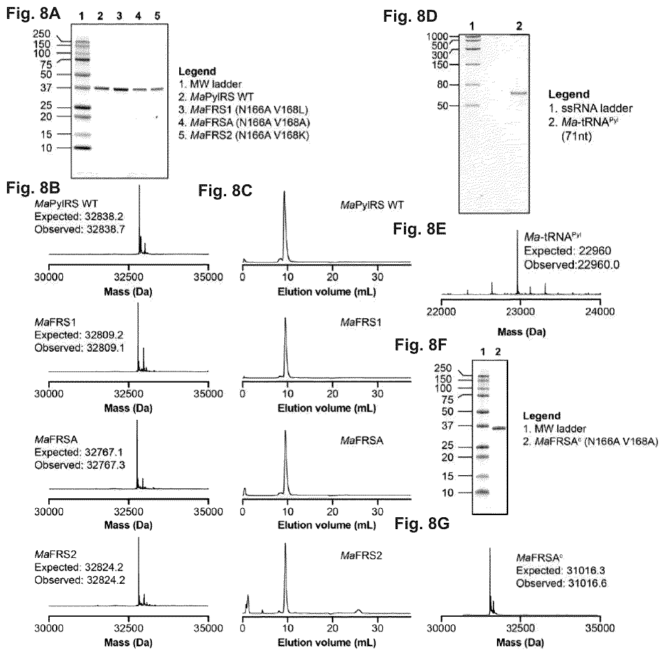

FIGS. 9A-9E. Analysis of tRNA acylation product mixtures obtained using MaPylRS, Ma-tRNA^{Pyl} and monomer 1 as described. FIG. 9A, Total ion count and FIG. 9B, UV absorbance (260 nm) as a function of elution time. FIG. 9C, The raw MS deconvolution range represents the subset of the raw MS data used to determine the deconvoluted mass spectrum of each tRNA species (unacylated or monoacylated). The major ion identified with an asterisk is the most abundant charge state of the tRNA species used for quantification. FIG. 9D, Deconvoluted mass spectra generated from the data in (FIG. 9C). FIG. 9E, Extracted ion chromatograms of the major ions of each tRNA species. The peak corresponding to each tRNA species is noted with an asterisk. The EICs were integrated and the area under the curve (A) was used to determine the overall tRNA acylation yield according to the equation: yield=[(A_{mono-acylated}+A_{di-acylated})/(A_{unacylated}+A_{mono-acylated}+A_{di-acylated}). The yield shown in this figure is from a representative sample. Average yields from three technical replicates are displayed in Extended Data Table 3.

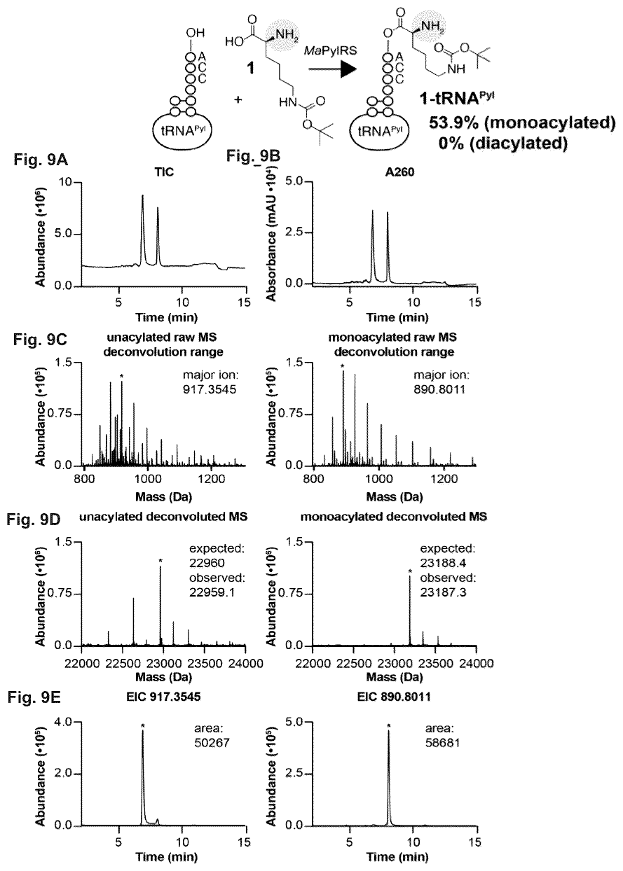

FIGS. 10A-10E. Analysis of tRNA product mixtures obtained using MaPylRS, Ma-tRNA^{Pyl} and monomer 2 as described. Please refer to the legend for Extended Data FIGS. 9A-9E for descriptions of FIGS. 10A-10E.

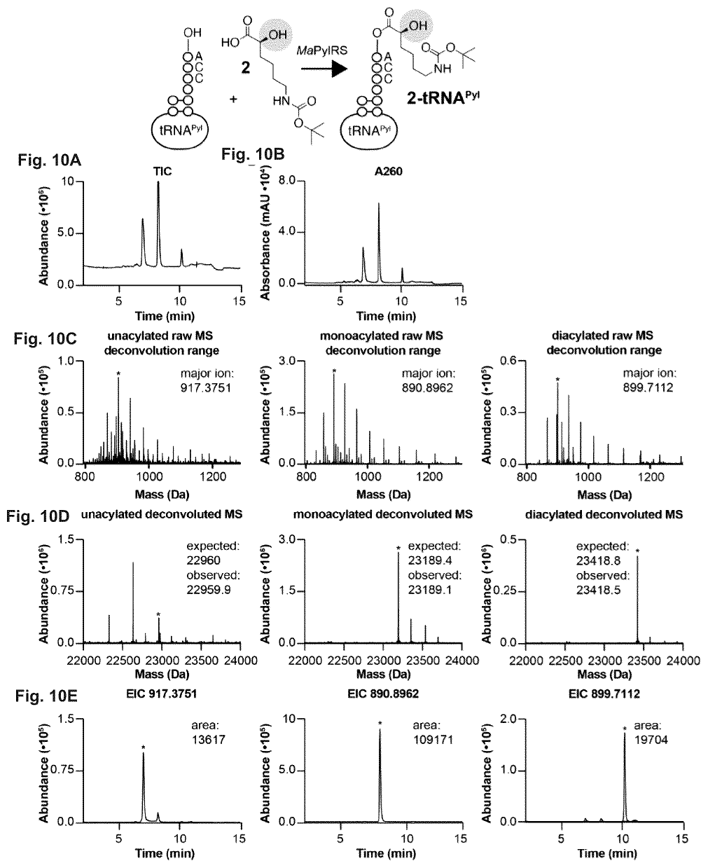

FIGS. 11A-11E. Analysis of tRNA product mixtures obtained using MaPylRS, Ma-tRNA^{Pyl} and monomer 3 as described. Please refer to the legend for Extended Data FIGS. 9A-9E for descriptions of FIGS. 11A-11E.

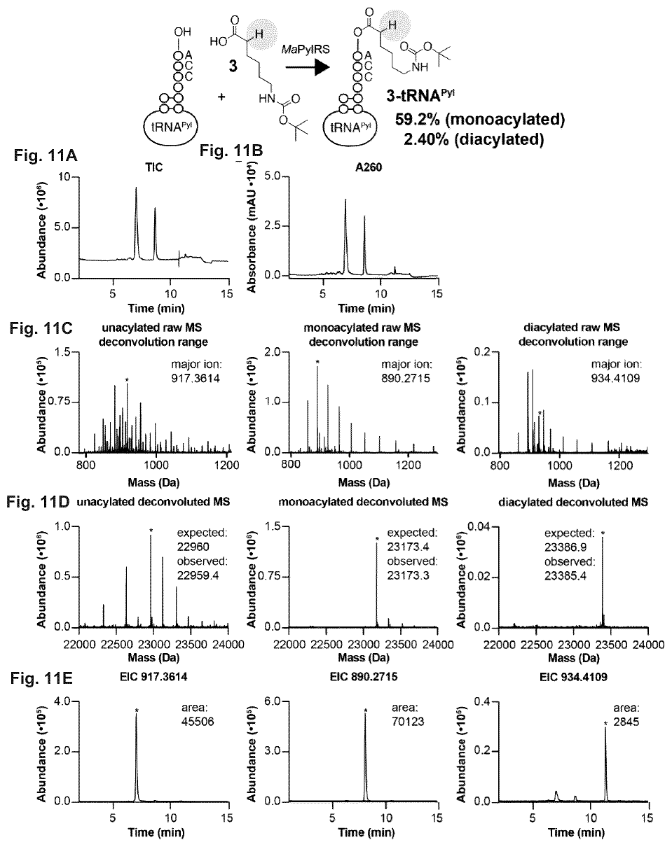

FIGS. 12A-12E. Analysis of tRNA product mixtures obtained using MaPylRS, Ma-tRNA^{Pyl} and monomer 5 as described. Please refer to the legend for Extended Data FIGS. 9A-9E for descriptions of FIGS. 12A-12E.

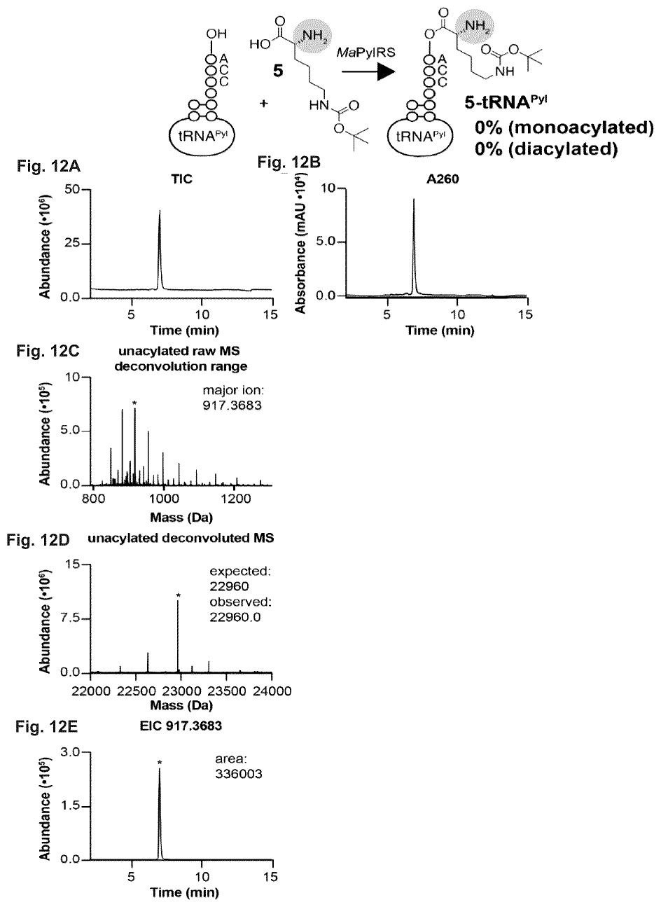

FIGS. 13A-13E. Analysis of tRNA product mixtures obtained using MaPylRS, Ma-tRNA^{Pyl} and monomer 16 as described. Please refer to the legend for Extended Data FIGS. 9A-9E for descriptions of FIGS. 13A-13E. In FIG. 13C, the major ion for both the base mass and the decarboxylation product are listed. In FIG. 13D, the decarboxylation product mass is denoted by a D. The areas under the curve in FIG. 13E for the base and decarboxylation product masses were combined to calculate the overall acylation yield.

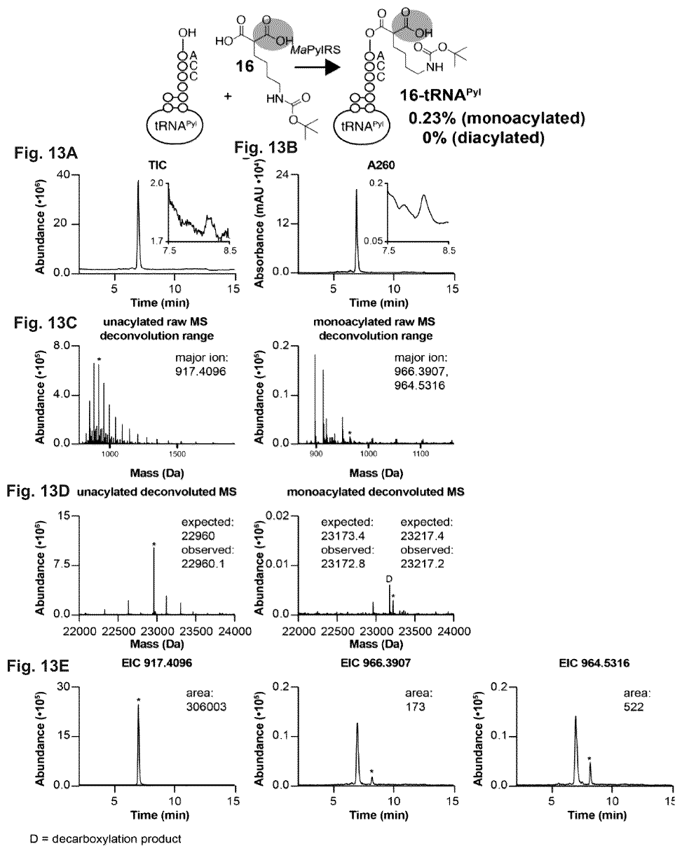

FIGS. 14A-14E. Analysis of tRNA product mixtures obtained using MaFRS1, Ma-tRNA^{Pyl} and monomer 7 as described. Please refer to the legend for Extended Data FIGS. 9A-9E for descriptions of FIGS. 14A-14E.

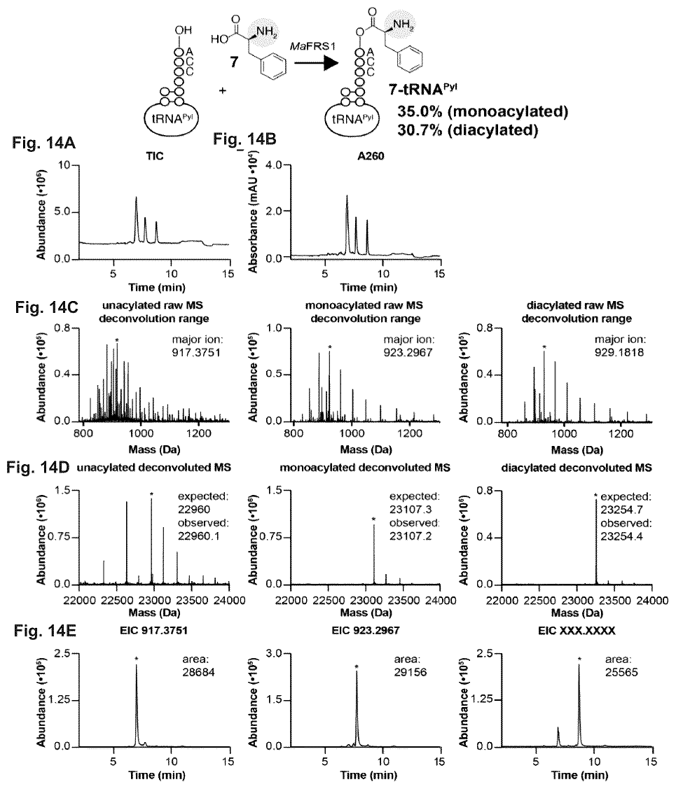

FIGS. 15A-15E. Analysis of tRNA product mixtures obtained using MaFRS1, Ma-tRNA^{Pyl} and monomer 8 as described. Please refer to the legend for Extended Data FIGS. 9A-9E for descriptions of FIGS. 15A-15E.

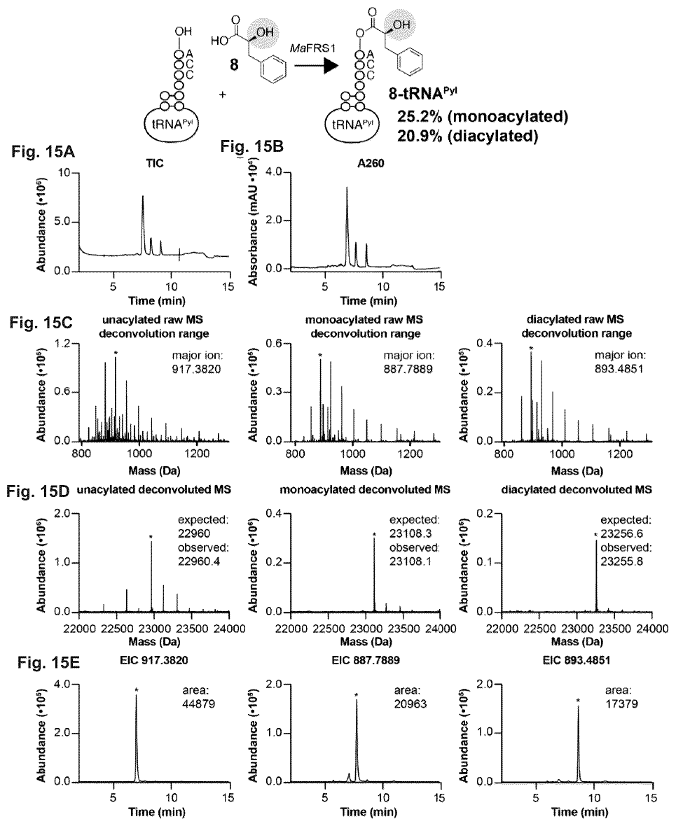

FIGS. 16A-16E. Analysis of tRNA product mixtures obtained using MaFRS1, Ma-tRNA^{Pyl} and monomer 9 as described. Please refer to the legend for Extended Data FIGS. 9A-9E for descriptions of FIGS. 16A-16E.

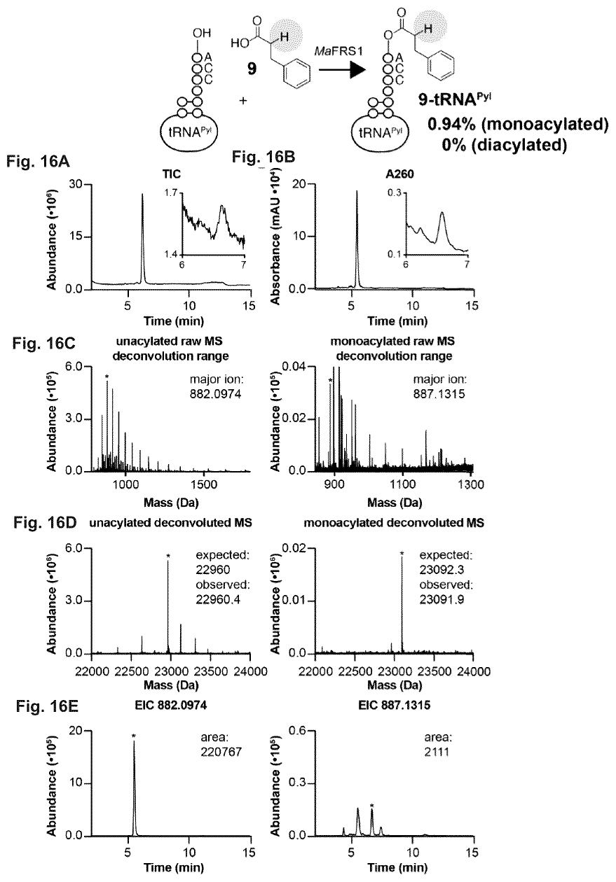

FIGS. 17A-17E. Analysis of tRNA product mixtures obtained using MaFRS1, Ma-tRNA^{Pyl} and monomer 10 as described. Please refer to the legend for Extended Data FIGS. 9A-9E for descriptions of FIGS. 17A-17E.

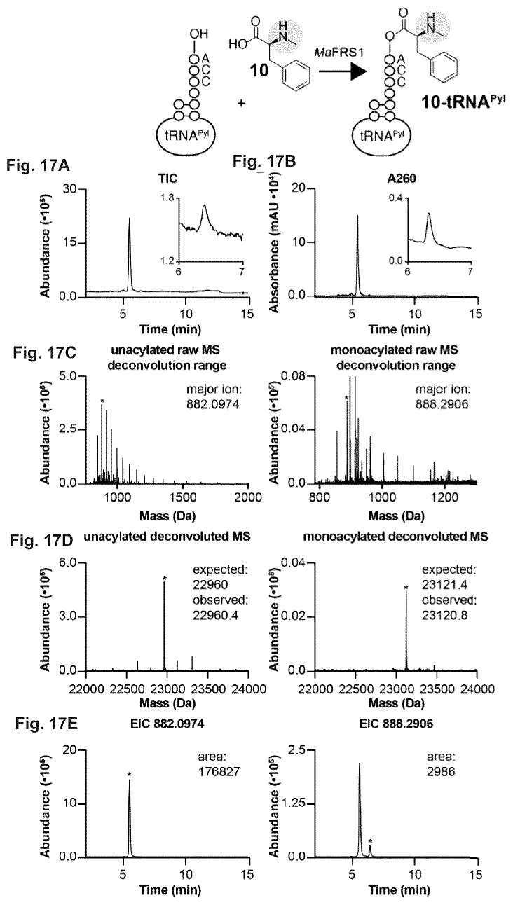

FIGS. 18A-18E. Analysis of tRNA product mixtures obtained using MaFRS1, Ma-tRNA^{Pyl} and monomer 11 as described. Please refer to the legend for Extended Data FIGS. 9A-9E for descriptions of FIGS. 18A-18E.

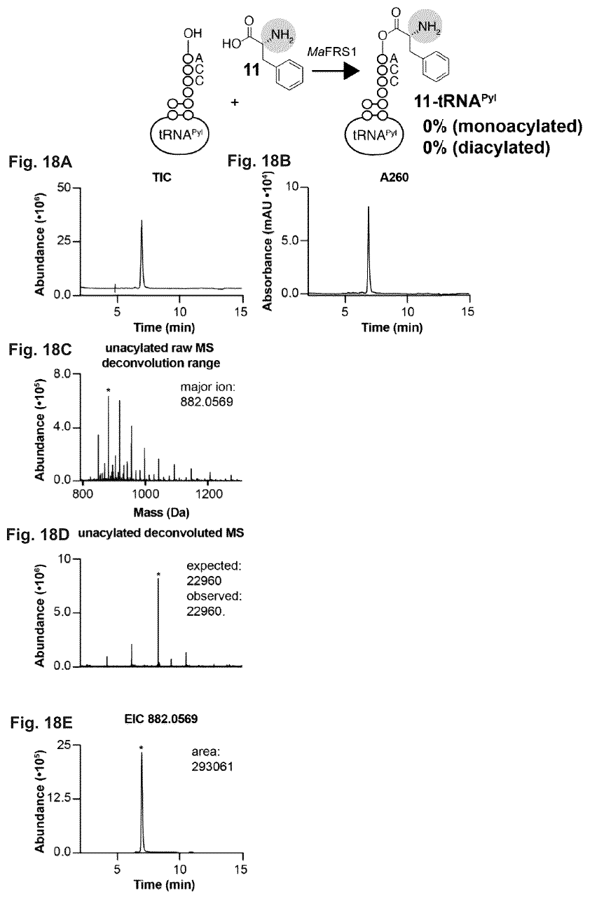

FIGS. 19A-19E. Analysis of tRNA product mixtures obtained using MaFRS1, Ma-tRNA^{Pyl} and monomer 13 as described. Please refer to the legend for Extended Data FIGS. 9A-9E for descriptions of FIGS. 19A-19E.

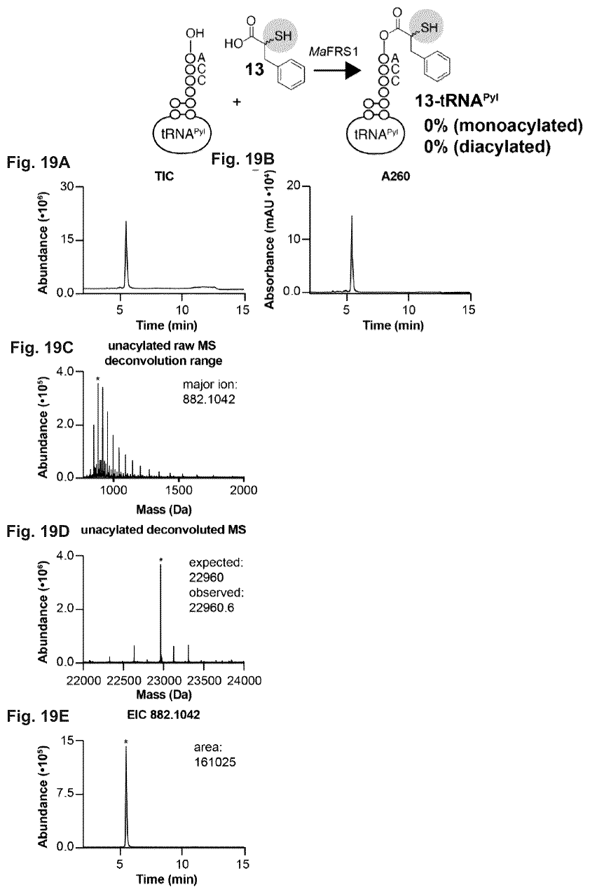

FIGS. 20A-20E. Analysis of tRNA product mixtures obtained using MaFRS1, Ma-tRNA^{Pyl} and monomer 13 as described. Please refer to the legend for Extended Data FIGS. 9A-9E for descriptions of FIGS. 20A-20E. In this case, acylation was performed using MaFRS1:Ma-tRNA^{Pyl} ratio of 1:2.

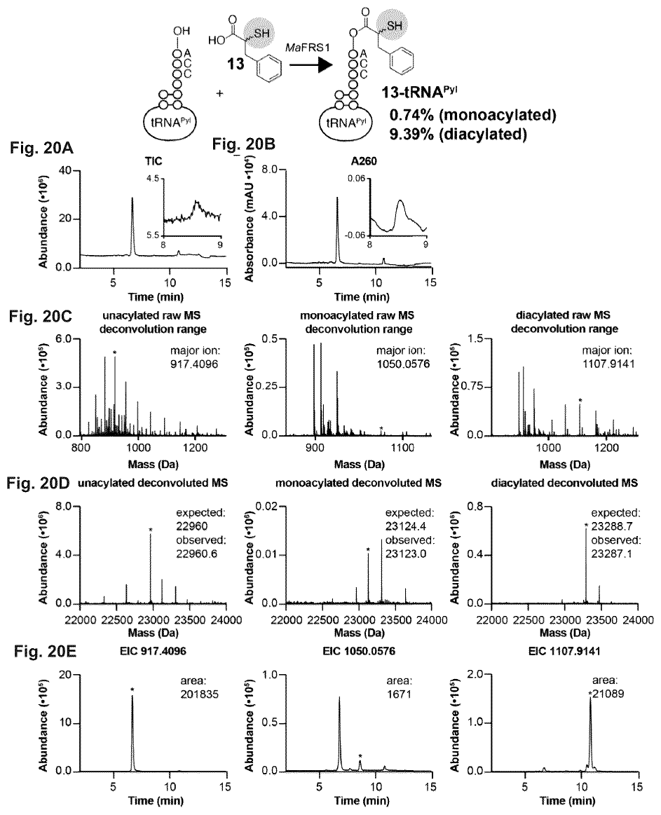

FIGS. 21A-21E. Analysis of tRNA product mixtures obtained using MaFRS1, Ma-tRNA^{Pyl} and monomer 14 as described. Please refer to the legend for Extended Data FIGS. 9A-9E for descriptions of FIGS. 21A-21E and the legend of S3E for a note on the decarboxylation products observed in the mass spectra.

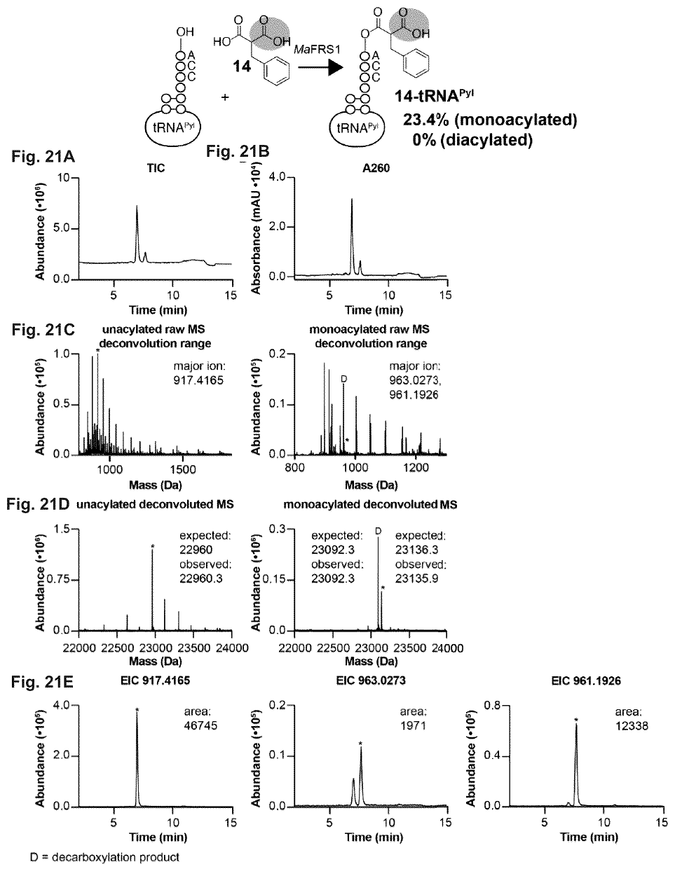

FIGS. 22A-22E. Analysis of tRNA product mixtures obtained using MaFRS1. Ma-tRNA^{Pyl} and monomer 15 as described. Please refer to the legend for Extended Data FIGS. 9A-9E for descriptions of FIGS. 22A-22E.

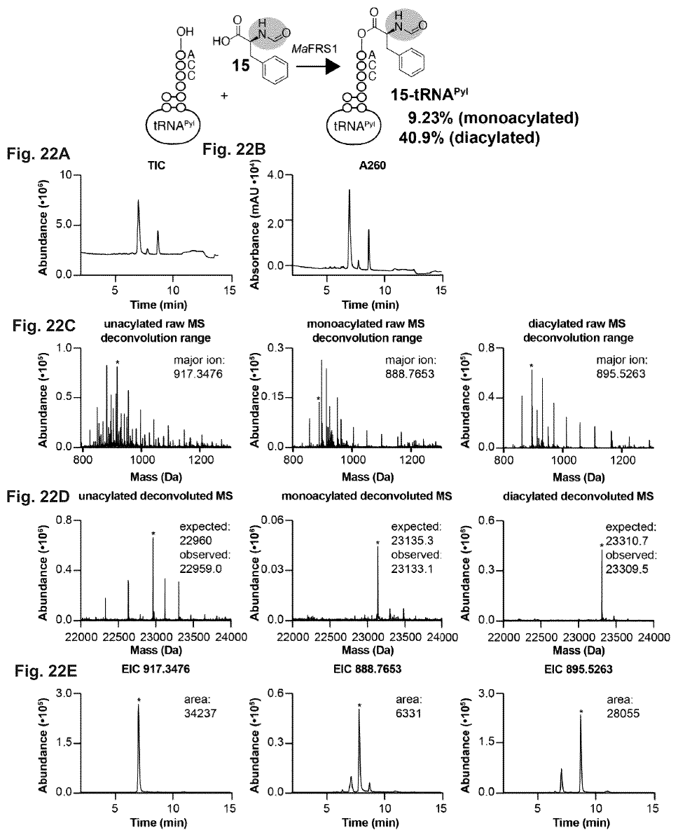

FIGS. 23A-23E. Analysis of tRNA product mixtures obtained using MaFRS1, Ma-tRNA^{Pyl} and monomer 17 as described. Please refer to the legend for Extended Data FIGS. 9A-9E for descriptions of FIGS. 23A-23E and the legend of S3E for a note on the decarboxylation products observed in the mass spectra.

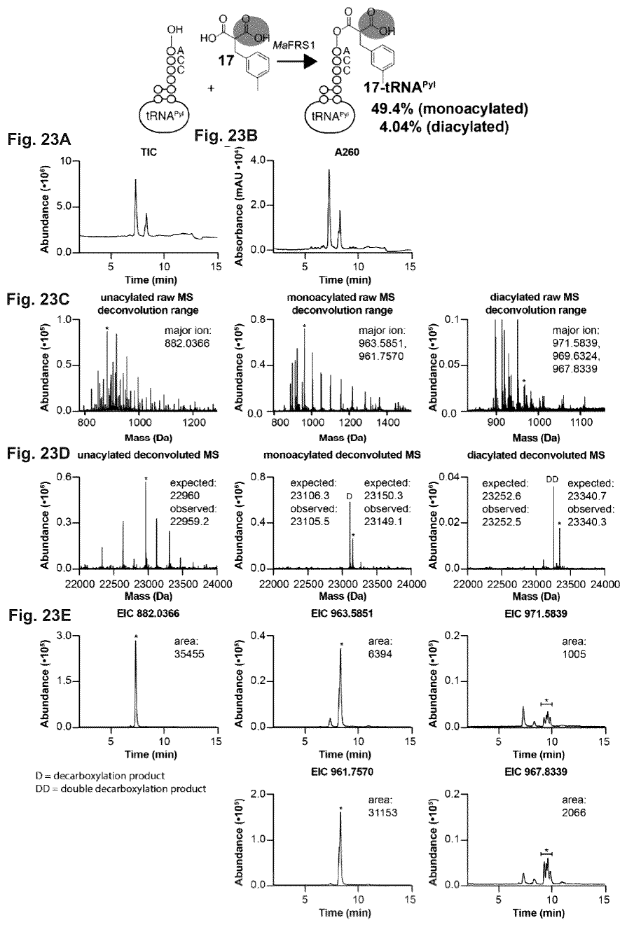

FIGS. 24A-24E. Analysis of tRNA product mixtures obtained using MaFRS1, Ma-tRNA^{Pyl} and monomer 18 as described. Please refer to the legend for Extended Data FIGS. 9A-9E for descriptions of FIGS. 24A-24E and the legend of S3E for a note on the decarboxylation products observed in the mass spectra.

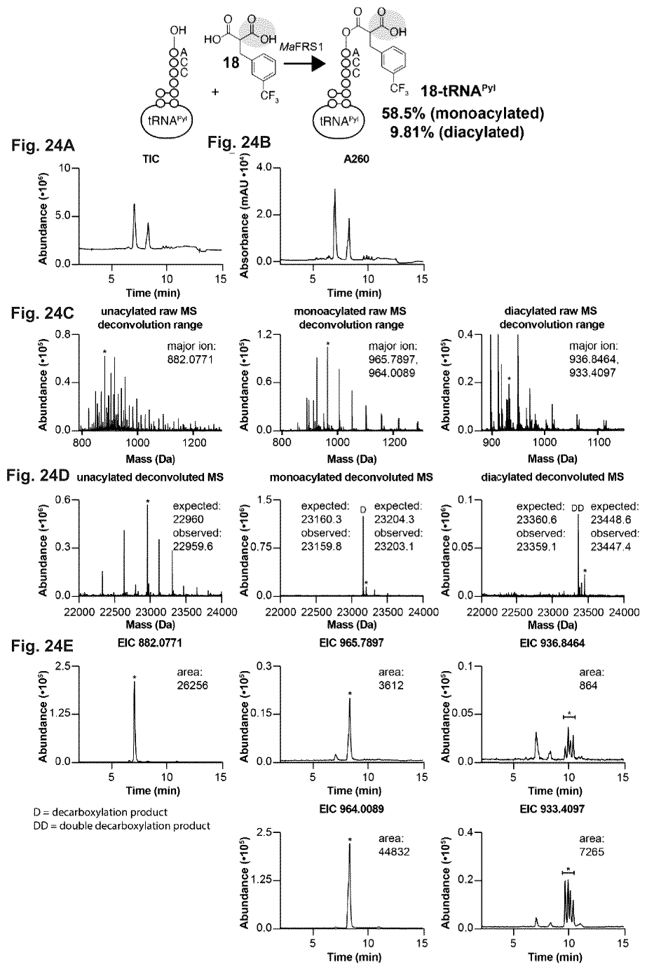

FIGS. 25A-25E. Analysis of tRNA product mixtures obtained using MaFRS1, Ma-tRNA^{Pyl} and monomer 19 as described. Please refer to the legend for Extended Data FIGS. 9A-9E for descriptions of FIGS. 25A-25E and a note on the decarboxylation products observed in the mass spectra.

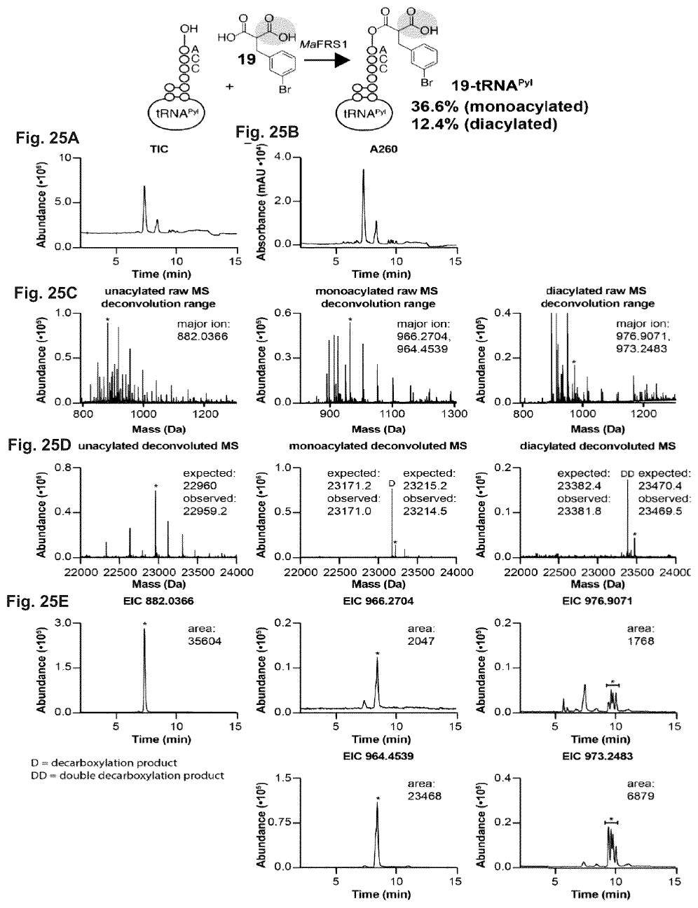

FIGS. 26A-26E. Analysis of tRNA product mixtures obtained using MaFRS2. Ma-tRNA^{Pyl} and monomer 7 as described. Please refer to the legend for Extended Data FIGS. 9A-9E for descriptions of FIGS. 26A-26E.

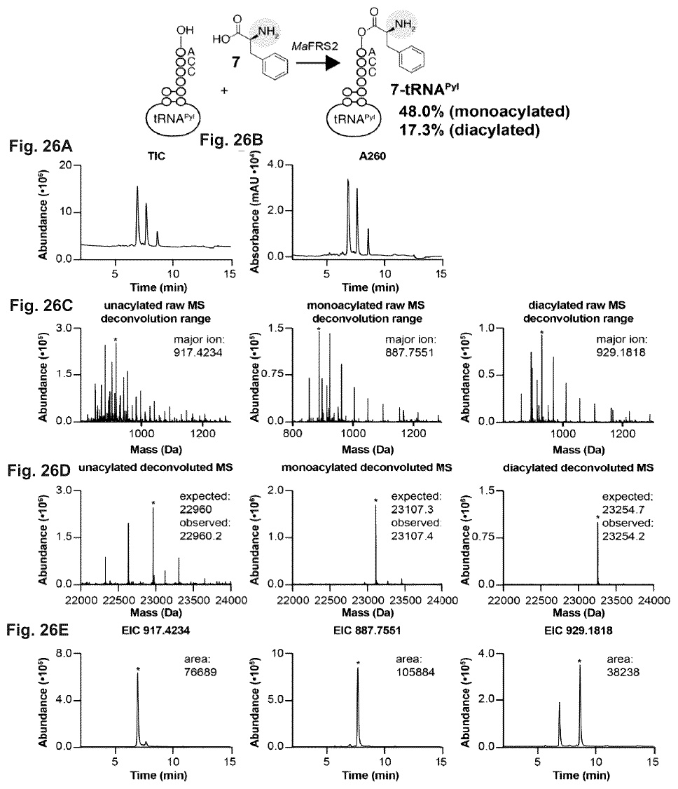

FIGS. 27A-27E. Analysis of tRNA product mixtures obtained using MaFRS2, Ma-tRNA^{Pyl} and monomer 8 as described. Please refer to the legend for Extended Data FIGS. 9A-9E for descriptions of FIGS. 27A-27E.

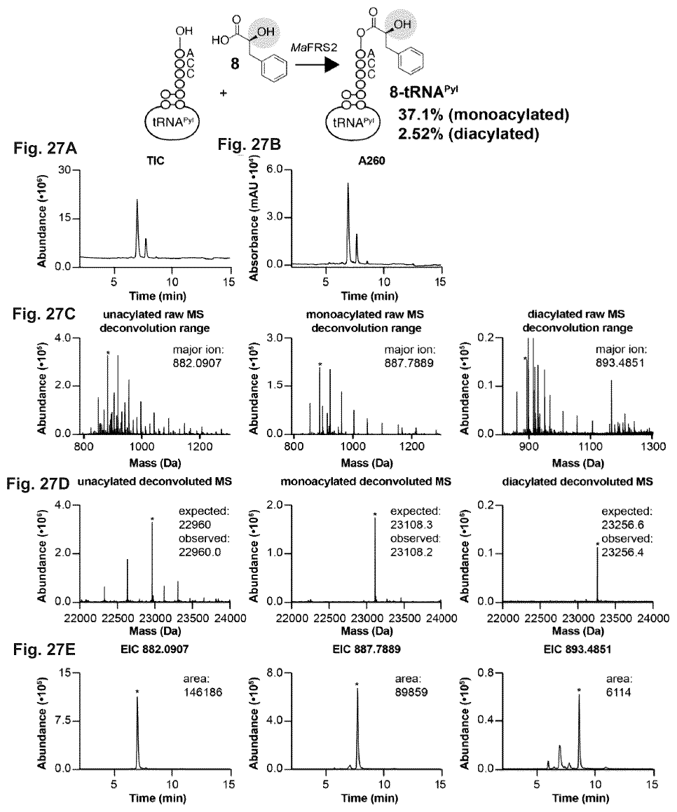

FIGS. 28A-28E. Analysis of tRNA product mixtures obtained using MaFRS2, Ma-tRNA^{Pyl} and monomer 9 as described. Please refer to the legend for Extended Data FIGS. 9A-9E for descriptions of FIGS. 28A-28E.

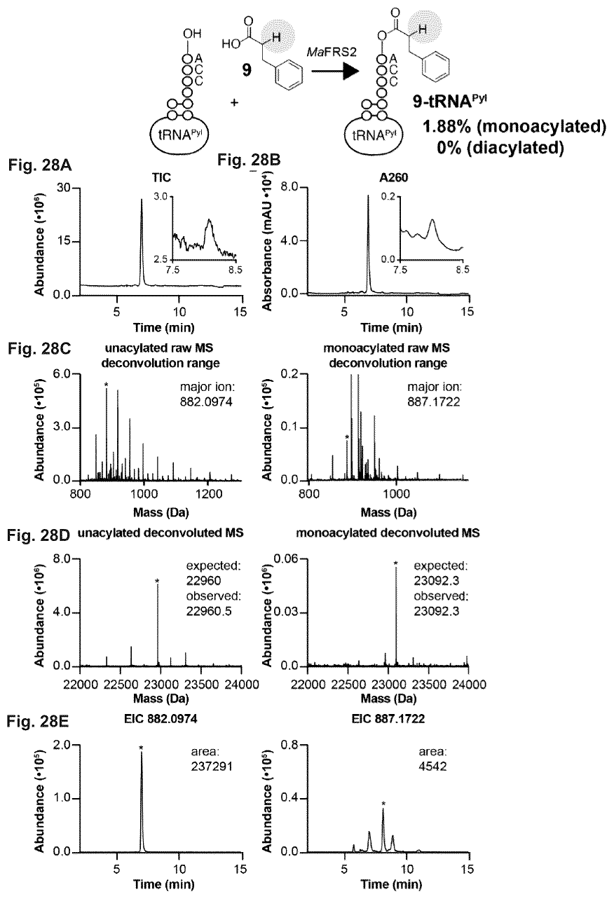

FIGS. 29A-29E. Analysis of tRNA product mixtures obtained using MaFRS2, Ma-tRNA^{Pyl} and monomer 10 as described. Please refer to the legend for Extended Data FIGS. 9A-9E for descriptions of FIGS. 29A-29E.

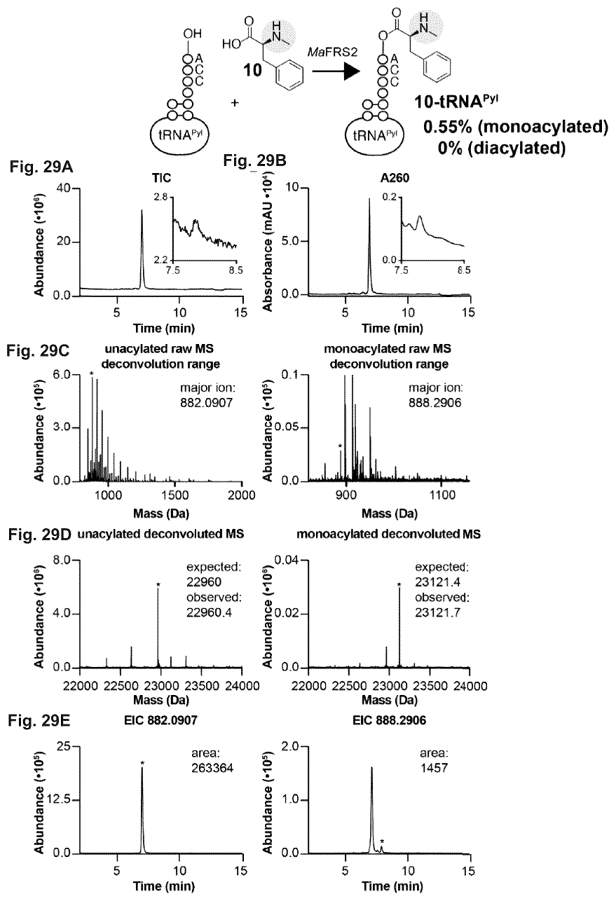

FIGS. 30A-30E. Analysis of tRNA product mixtures obtained using MaFRS2, Ma-tRNA^{Pyl} and monomer 11 as described. Please refer to the legend for Extended Data FIGS. 9A-9E for descriptions of FIGS. 30A-30E.

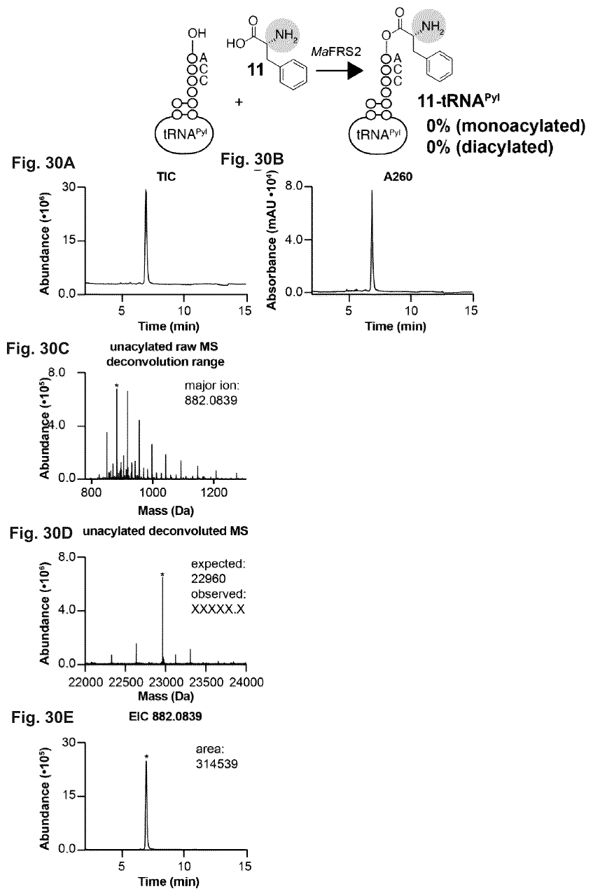

FIGS. 31A-31E. Analysis of tRNA product mixtures obtained using MaFRS2, Ma-tRNA^{Pyl} and monomer 13 as described. Please refer to the legend for Extended Data FIGS. 9A-9E for descriptions of FIGS. 31A-31E.

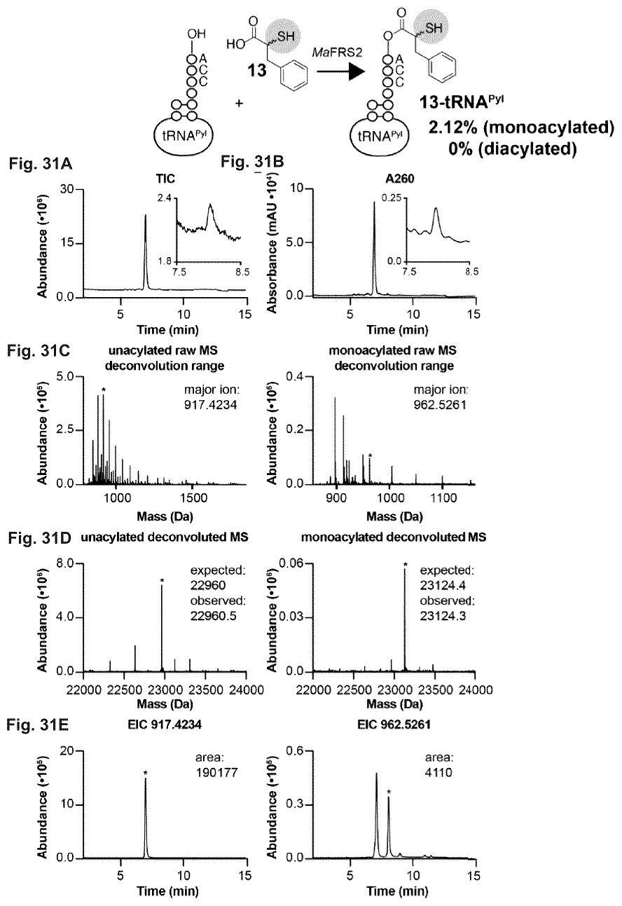

FIGS. 32A-32E. Analysis of tRNA product mixtures obtained using MaFRS2, Ma-tRNA^{Pyl} and monomer 14 as described. Please refer to the legend for Extended Data FIGS. 9A-9E for descriptions of FIGS. 32A-32E and a note on the decarboxylation products observed in the mass spectra.

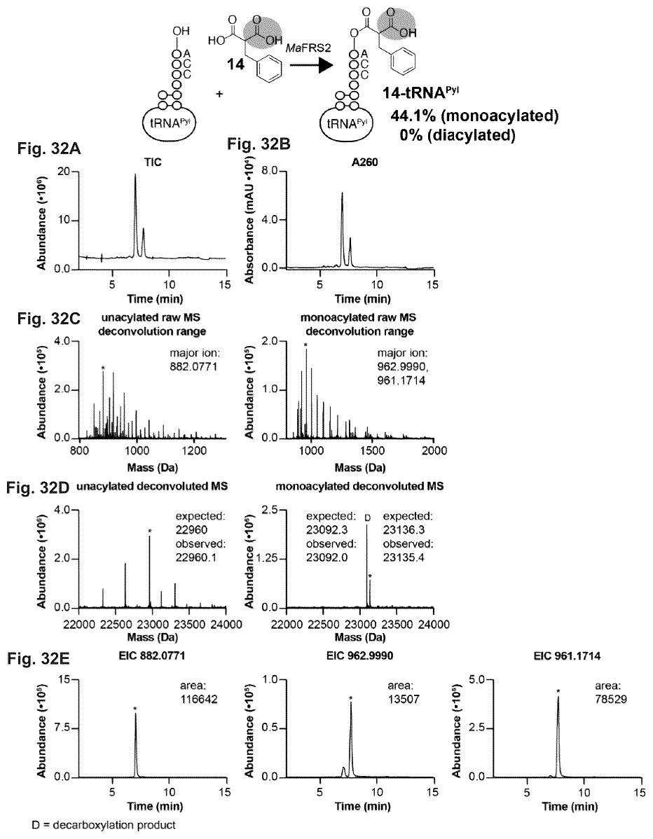

FIGS. 33A-33E. Analysis of tRNA product mixtures obtained using MaFRS2, Ma-tRNA^{Pyl} and monomer 15 as described. Please refer to the legend for Extended Data FIGS. 9A-9E for descriptions of FIGS. 33A-33E.

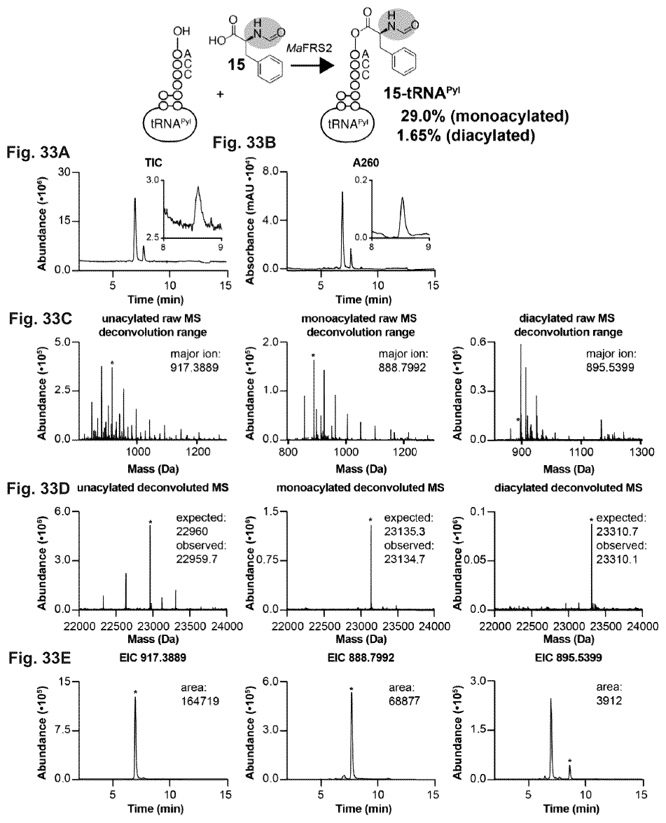

FIGS. 34A-34E. Analysis of tRNA product mixtures obtained using MaFRS2, Ma-tRNA^{Pyl} and monomer 17 as described. Please refer to the legend for Extended Data FIGS. 9A-9E for descriptions of FIGS. 34A-34E and a note on the decarboxylation products observed in the mass spectra.

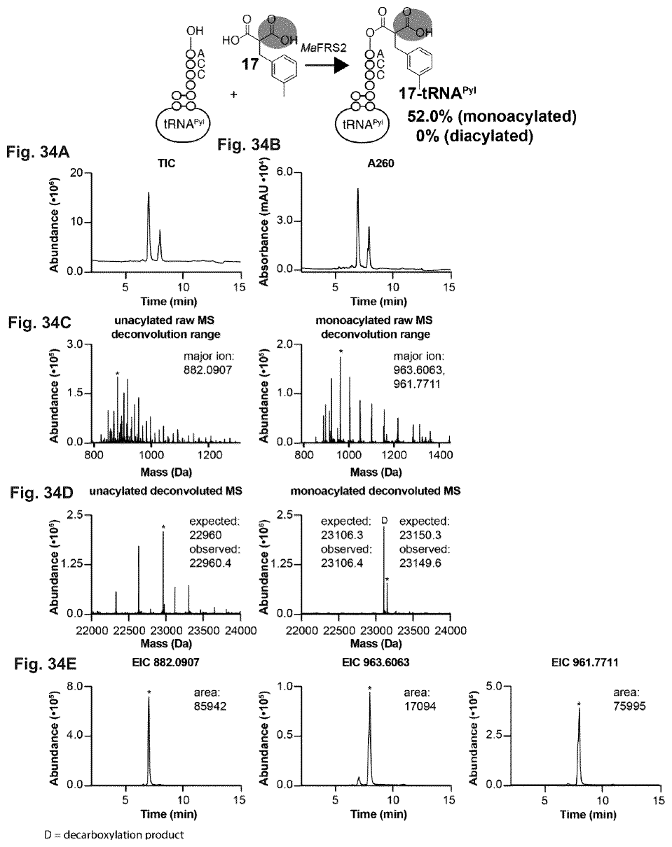

FIGS. 35A-35E. Analysis of tRNA product mixtures obtained using MaFRS2, Ma-tRNA^{Pyl} and monomer 18 as described. Please refer to the legend for Extended Data FIGS. 9A-9E for descriptions of FIGS. 35A-35E and a note on the decarboxylation products observed in the mass spectra.

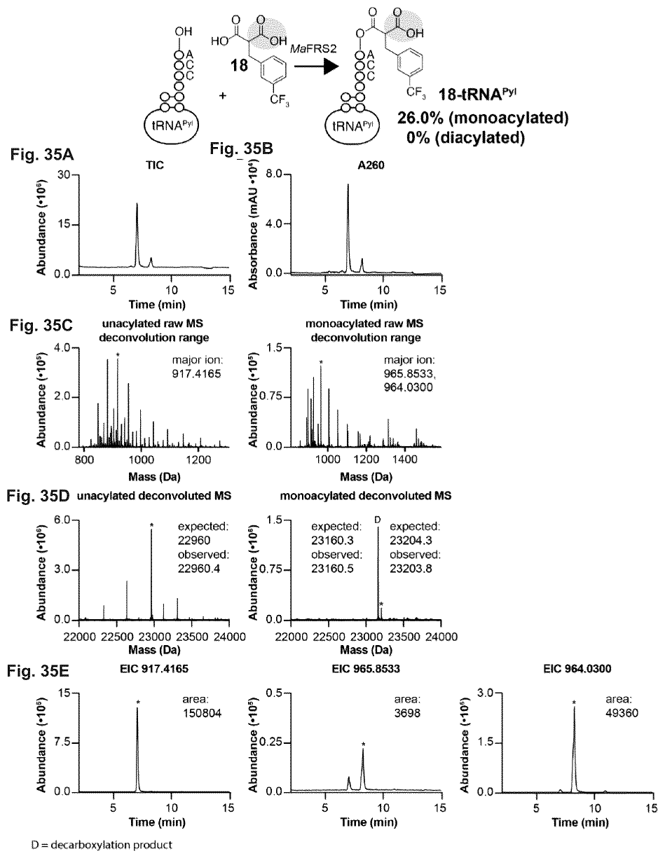

FIGS. 36A-36E. Analysis of tRNA product mixtures obtained using MaFRS2, Ma-tRNA^{Pyl} and monomer 19 as described. Please refer to the legend for Extended Data FIGS. 9A-9E for descriptions of FIGS. 36A-36E and a note on the decarboxylation products observed in the mass spectra.

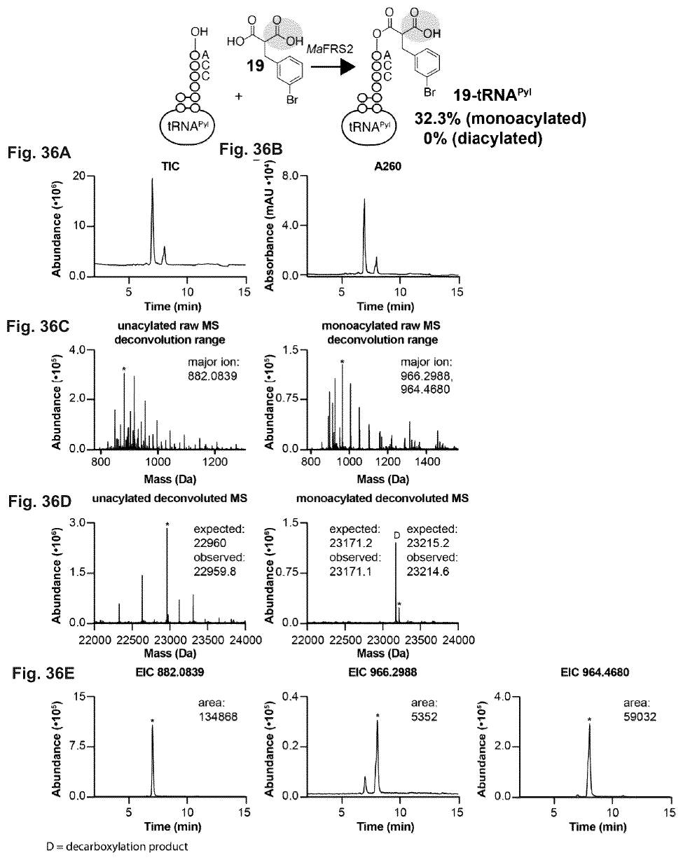

FIGS. 37A-37E. Analysis of tRNA product mixtures obtained using MaFRSA, Ma-tRNA^{Pyl} and monomer 7 as described. Please refer to the legend for Extended Data FIGS. 9A-9E for descriptions of FIGS. 37A-37E.

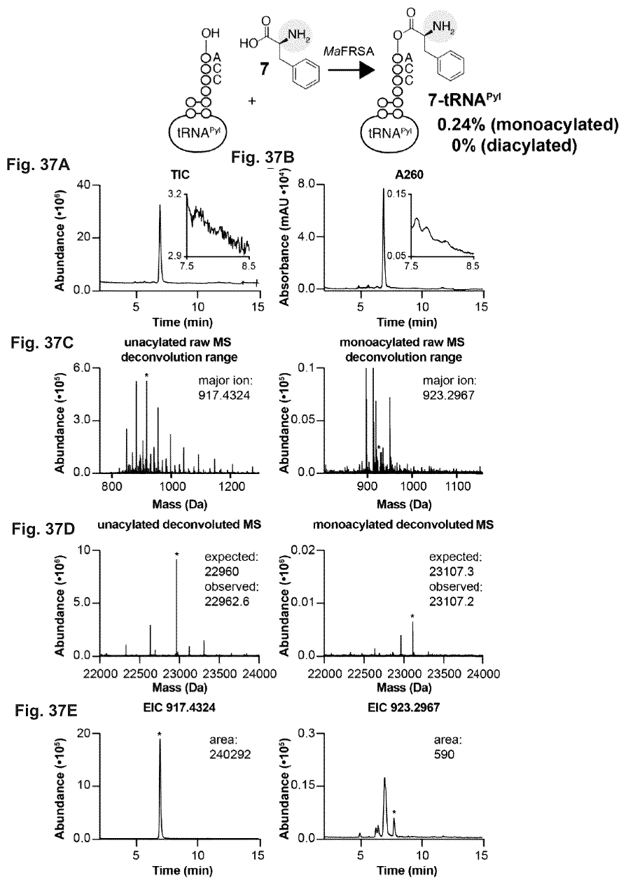

FIGS. 38A-38E. Analysis of tRNA product mixtures obtained using MaFRSA, Ma-tRNA^{Pyl} and monomer 17 as described. Please refer to the legend for Extended Data FIGS. 9A-9E for descriptions of FIGS. 38A-38E and a note on the decarboxylation products observed in the mass spectra.

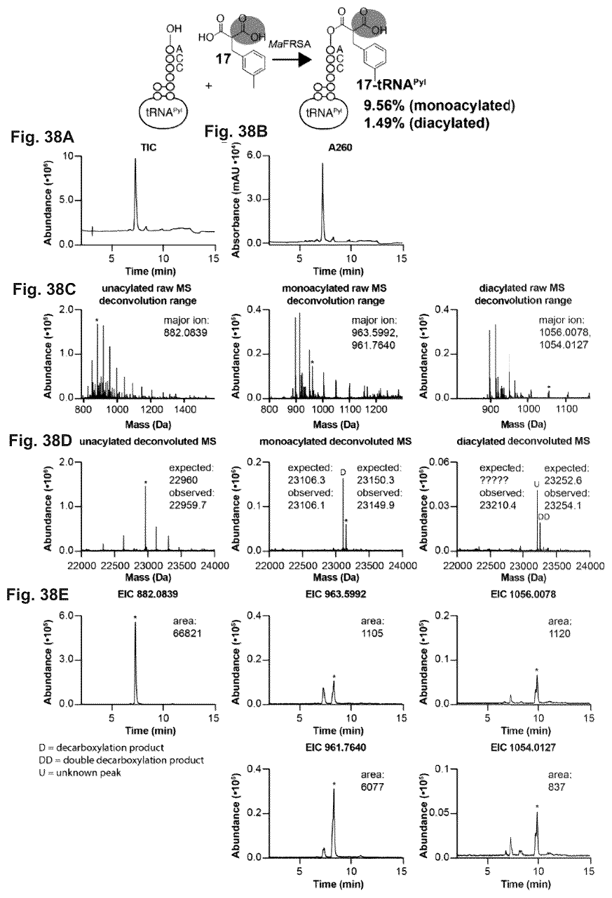

FIGS. 39A-39E. Analysis of tRNA product mixtures obtained using MaFRSA, Ma-tRNA^{Pyl} and monomer 18 as described. Please refer to the legend for Extended Data FIGS. 9A-9E for descriptions of FIGS. 39A-39E and a note on the decarboxylation products observed in the mass spectra.

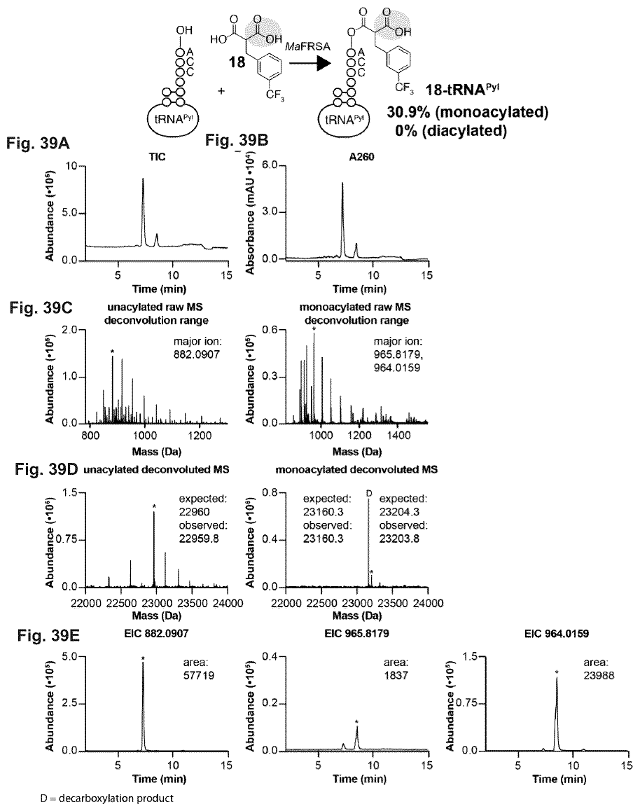

FIGS. 40A-40E. Analysis of tRNA product mixtures obtained using MaFRSA, Ma-tRNA^{Pyl} and monomer 19 as described. Please refer to the legend for Extended Data FIGS. 9A-9E for descriptions of FIGS. 40A-40E and a note on the decarboxylation products observed in the mass spectra.

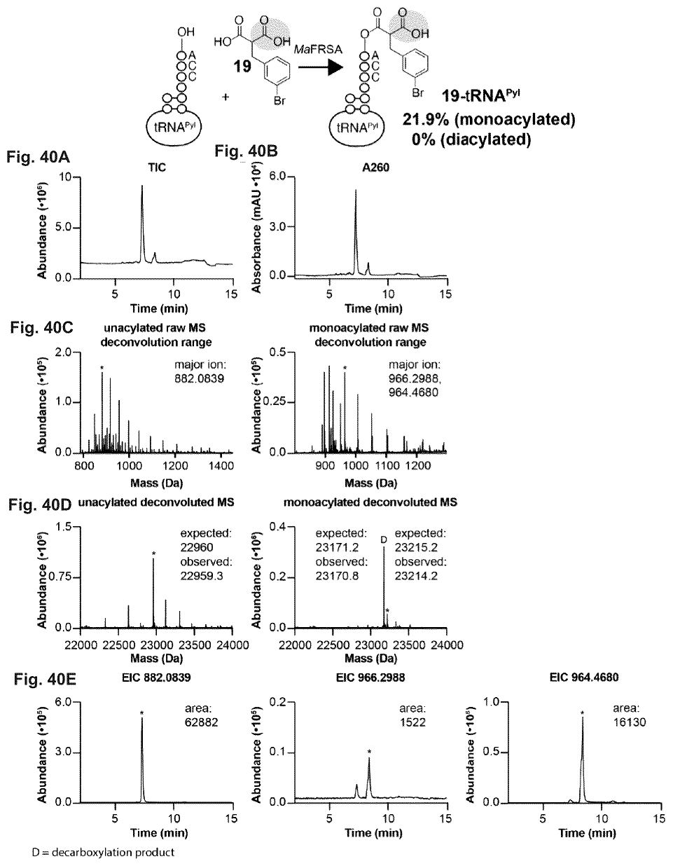

FIGS. 41A-41E. Analysis of tRNA product mixtures obtained using MaFRSA, Ma-tRNA^{Pyl} and monomers 7 and 18 as described. Please refer to the legend for Extended Data FIGS. 9A-9E for descriptions of FIGS. 41A-41E and a note on the decarboxylation products observed in the mass spectra. Only the acylation product of 18 and Ma-tRNA^{Pyl} is observed.

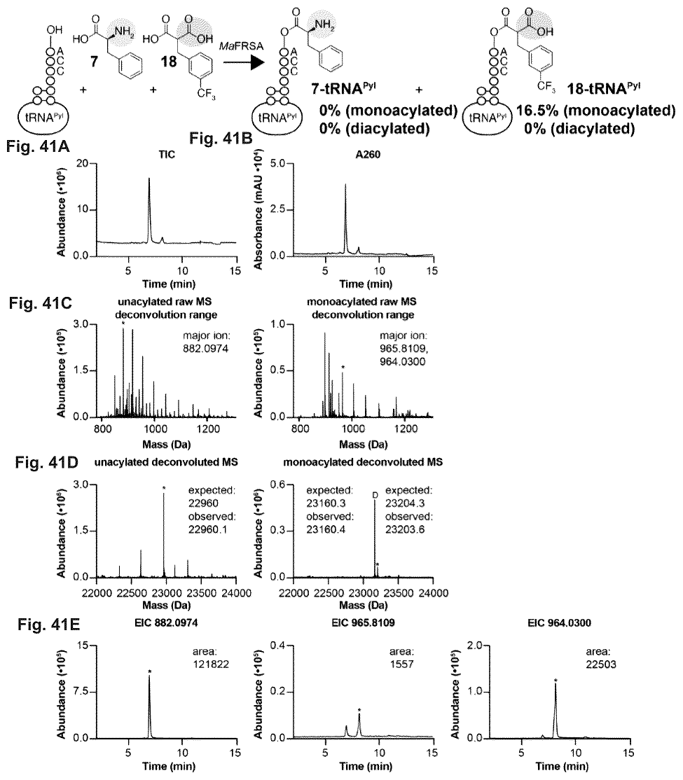

FIG. 42. Additional RNAse A assay experiments analyzed by LC-HRMS. The enzyme and substrate are noted in the top left of each plot. These data provide evidence that MaPylRS accepts 16 but not 5 as a substrate. Similarly, MaFRS1 and MaFRS2 accept 17, 18, and 19, but not 11.

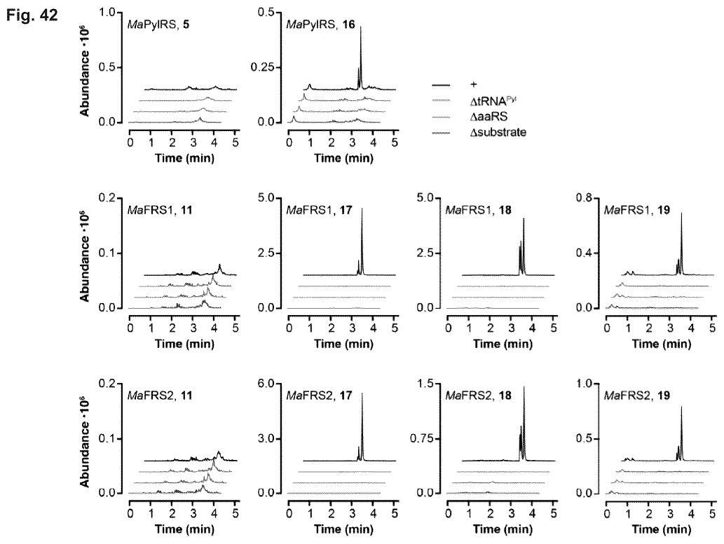

FIGS. 43A-43C. FIG. 43A, Malonic acid monomers used in this study. FIG. 43B, Left, 24a-d: malonyl adenosine nucleoside that is formed when malonyl-tRNA is digested by RNAse A; right, 25: decarboxylation product of malonyl-adenosine nucleoside. FIG. 43C, LC-HRMS analysis of Ma-tRNA^{Pyl} acylation reactions after RNAse A digestion. Reactions were performed as described. The EIC for the malonyl product 24 as a mixture of 2′ and 3′ isomers (two pairs of diastereomers) (pink, top) shows the expected peaks whereas the EIC for the decarboxylation product 25, also as a mixture of 2′ and 3′ isomers (black, bottom) shows that the decarboxylation product is absent in all cases except the MaPylRS-catalyzed acylation of Ma-tRNA^{Pyl} with monomer 16. These data provide evidence that the decarboxylation of malonates charged to Ma-tRNAPyl observed in the intact tRNA mass analysis occurs during either the workup or the LC-MS itself.

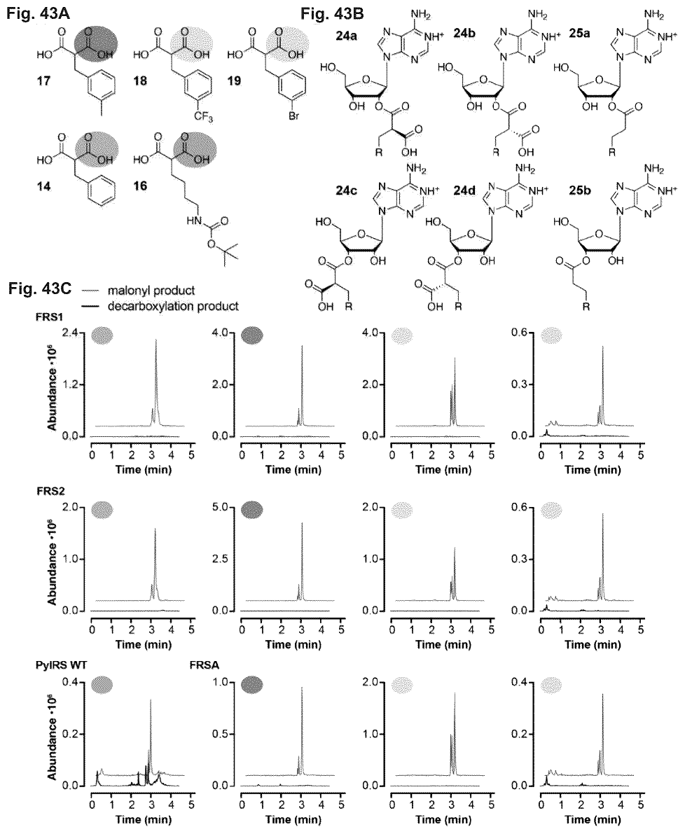

FIGS. 44A-44F. Ligand densities and recognition of meta-CF₃-2-BMA by MaFRSA. Electron density shown from the 2F₀-F_{c} map contoured at 1σ for meta-CF₃-2-BMA bound to chain A (FIG. 44A) and chain B (FIG. 44B), and AMP-PNP bound to chain A (FIG. 44C) and chain B (FIG. 44D). Expanded view of MaFRSA recognition of meta-CF₃-2-BMA in chain A (light purple, FIG. 44E) and chain B (dark purple, FIG. 44F) with additional active site residues displayed.

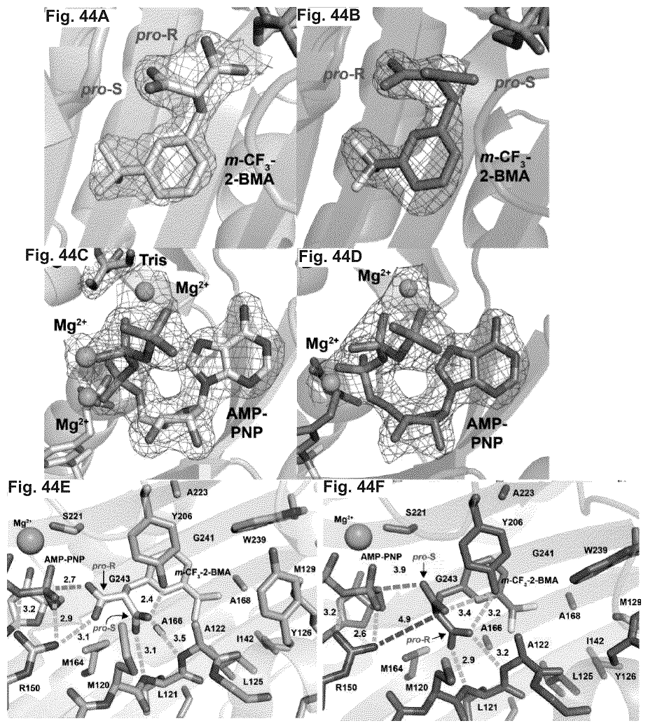

FIGS. 45A-45D. Structural recognition of meta-CF₃-2-BMA by MaFRSA and β7-β8 loop positioning in comparison with published PylRS structures. FIG. 45A, Alignment of MmIFRS (N346S/C348Q, yellow, PDB: 4TQD)⁵³ bound to 3-I-Phe and AMP-PNP and MaFRSA bound to meta-CF₃-2-BMA and AMP-PNP chain A (light purple) illustrating similar interactions between substrate carboxylate and backbone amides. The flexible β7-β8 loop ranges between unstructured, an open conformation, and a closed conformation across PylRS structures. FIG. 45B, MaFRSA bound to meta-CF₃-2-BMA and AMP-PNP (light purple), wild-type MaPylRS apo (green, PDB: 6JP2)⁵⁵, wild-type MmPylRS bound to PylK and AMP-PNP (blue, PDB: 2ZCE)³⁴, and wild-type MmPylRS bound to pyrrolysyl-adenylate (brown, PDB: 2Q7H)⁵⁶. Chain A (light purple, FIG. 45C) exhibits higher B-factors for the β7-β8 loop than chain B (dark purple, FIG. 45D) indicated by dark blue and thin ribbon for lower B-factors and dark red and thick ribbon for higher B-factors.

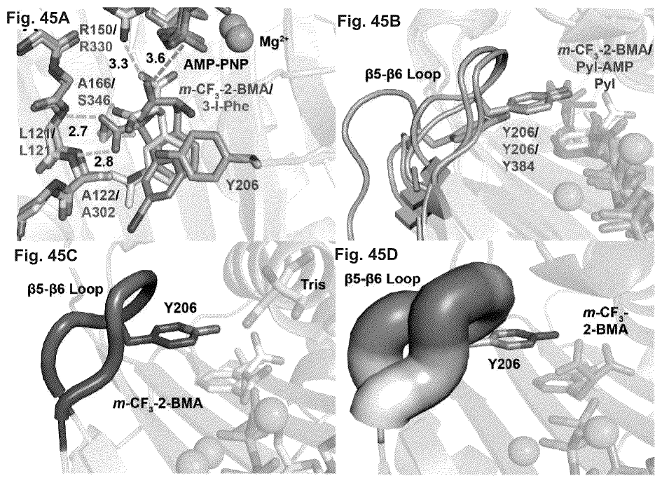

FIGS. 46A-46B. Annotated maps of plasmids used for in vivo expression of sfGFP (FIGS. 6C-E). FIG. 46A, pMega plasmids used for MaPylRS or MaFRSA expression. FIG. 46B, Reporter plasmids used for expression of sfGFP with a TAG stop codon at position 200 or 151.

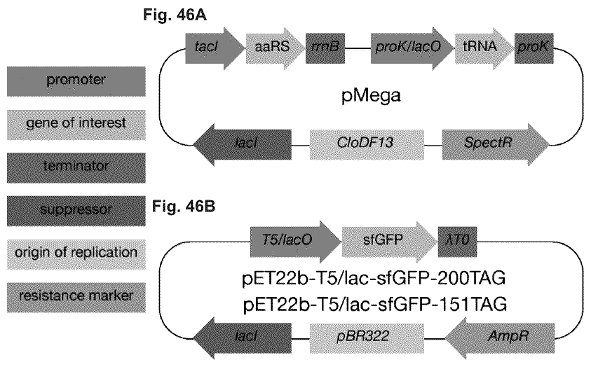

FIGS. 47A-47B. Plate reader analysis of sfGFP expression in DH10B E. coli. Emission at 528 nm after 24 h sfGFP expression in E. coli DH10B cells harboring pEVOL or pMega plasmids encoding FIG. 47A, MaPylRS and Ma-tRNA^{Pyl} in the presence of 0 or 1 mM BocK (1) or α-OH BocK (2) or FIG. 47B, MaFRSA and Ma-tRNA^{Pyl} in the presence of 0 or 1 mM m-trifluoromethyl phenylalanine (20) or α-OH m-trifluoromethyl phenylalanine (21).

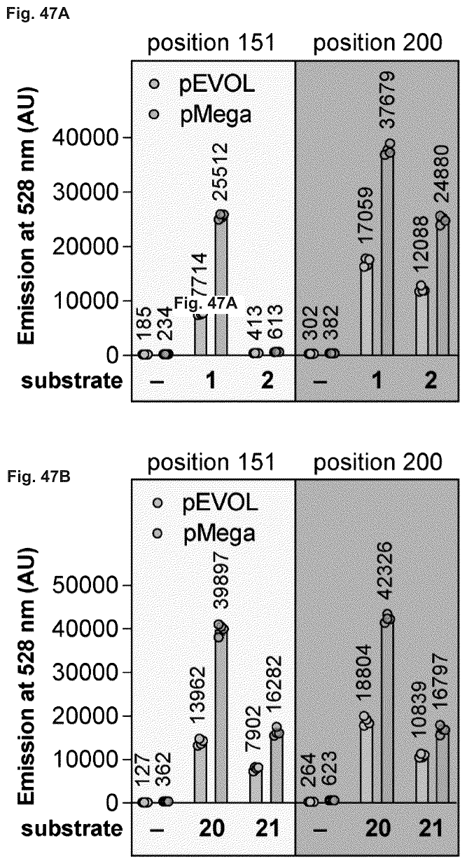

FIG. 48. Denaturing gel analysis of sfGFP variants. SDS-PAGE analysis of sfGFP variants expressed in DH10B or DH10B ΔaspC ΔtyrB in the presence of monomers 1, 2, 20, or 21. Abbreviations: “Pyl”=MaPylRS, “FA”=MaFRSA, “WT”=DH10b, “Δ”=DH10B ΔaspC ΔtyrB, “TB”=terrific broth, “DM”=defined media, +/−“OH—”=basic treatment or neutral control. Molecular weight ladder masses indicated at left in kDa.

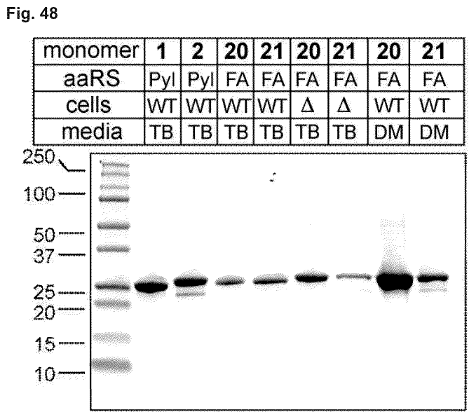

FIGS. 49A-49B. Sequence of sfGFP illustrating the peptide fragments obtained after digestion with GluC and their retention times. FIG. 49A, Fragments expected when sfGFP (SEQ ID NO:8) contains Y, BocK (1), or m-trifluoromethyl phenylalanine (20) at position 200. Digestion with Glu-C generates two overlapping peptides containing position 200, those encompassing residues 198-216 and 198-222. Both were used to quantify the composition at position 200. FIG. 49B, Fragments expected when sfGFP (SEQ ID NO:9) contains α-OH BocK (2) or α-OH m-trifluoromethyl phenylalanine (21) at position 200. In these cases, digestion with GluC exhibited additional cleavages at the proposed ester bond (presumably due to ester hydrolysis during work-up), generating two additional peptides containing position 200, encompassing residues 200-216 and 200-222. These peptides, in addition to those encompassing residues 198-216 and 198-222, were used to quantify the composition at residue 200. Colors from red to blue represent decreasing signal intensity. Retention times are indicated in the boxes illustrating the observed peptide fragments.

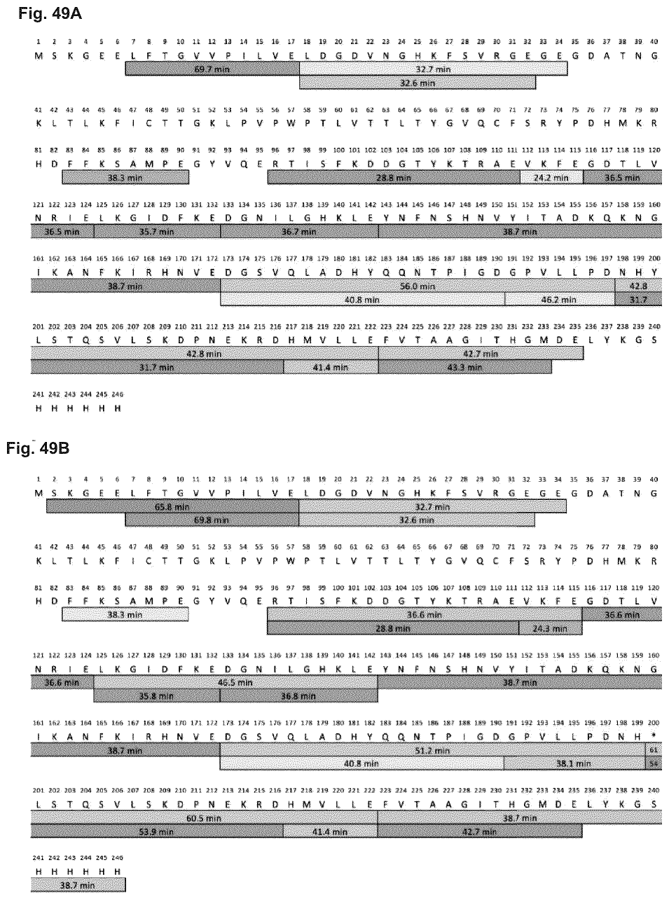

FIGS. 50A-50I. Mass spectrometry confirms the presence of an ester at position 200 of sfGFP. MS/MS identification of peptide 198-216, sequence: NHYLSTQSVLSKDPNEKRD (SEQ ID NO:10) from sfGFP expressed in DH10B cells containing FIG. 50A, tyrosine (WT); FIG. 50B, BocK (1); or FIG. 50C, α-OH BocK (2) at position 200. MS/MS identification of peptide 200-216, sequence: YLSTQSVLSKDPNEKRD (SEQ ID NO:11) resulting from sfGFP expressed in DH10B (FIG. 50D, FIG. 50E) or DH10B ΔaspC ΔtyrB (FIG. 50F, FIG. 50G) containing m-CF₃-Phe (20) (FIG. 50D, FIG. 50F) or α-OH m-CF₃-Phe (21) (FIG. 50E, FIG. 50G) at position 200. Peptides were generated by endoproteinase Glu-C digestion of sfGFP samples expressed with each indicated substrate. For fragment assignments, position 200 was considered as a tyrosine (in red) modified to have the correct mass. Abundance of α-NH₂ m-CF₃-Phe (FIG. 50H) and α-OH m-CF₃-Phe (FIG. 50I).

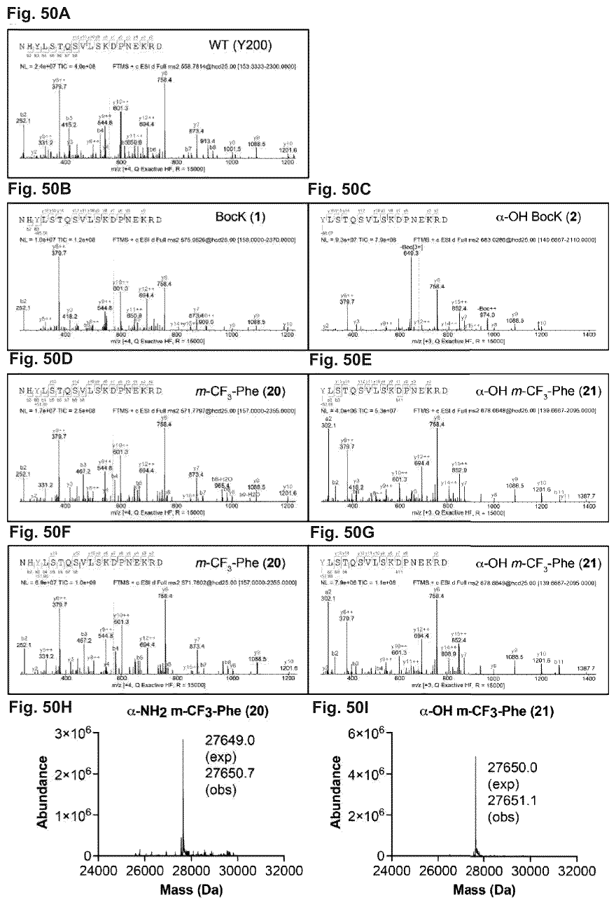

### DESCRIPTION OF PARTICULAR EMBODIMENTS OF THE INVENTION

Unless contraindicated or noted otherwise, in these descriptions and throughout this specification, the terms “a” and “an” mean one or more, the term “or” means and/or. It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein, including citations therein, are hereby incorporated by reference in their entirety for all purposes.

### Example: Expanding the Substrate Scope of PylRS Enzymes to Include Non-α-Amino Adds In Vitro and In Vivo

We hypothesized that the water-mediated α-amine recognition employed by MmPylRS could provide sufficient space and flexibility for substrates with less conservative substituents in place of the α-amine. Here we disclose that the tolerance of MmPylRS for substrates with multiple alternative substituents in place of the α-amine extends to Methanomethylophilus alvus PylRS (MaPylRS)^{34,35} as well as MaPylRS variants (MaFRS1 and MaFRS2) that recognize diverse L-phenylalanine derivatives³⁶. More importantly, MaFRS1 and MaFRS2 also accept phenylalanine derivatives with α-thio, N-formyl-L-α-amino, as well as an α-carboxyl substituent: 2-benzylmalonic acid. A final variant, MaFRSA³⁷, is selective for ring-substituted 2-benzylmalonate derivatives over L-Phe.

Malonates contain a 1,3-dicarbonyl unit that represents the defining backbone element of polyketide natural products, and after decarboxylation have the potential to support Claisen-type condensation within the PTC to form a carbon-carbon bond. Structural analysis of MaFRSA complexed with a meta-substituted 2-benzylmalonate derivative and a non-hydrolyzable ATP analogue reveals how the enzyme uses a novel pattern of hydrogen bonds to differentiate the two pro-chiral carboxylates in the substrate and accommodate the larger size and distinct electrostatics of an α-carboxyl substituent. In vitro translation studies confirm that tRNAs carrying α-thio, α-carboxyl, and N-formyl-L-α-amino acid monomers are effectively delivered to and accommodated by E. coli ribosomes. In vivo studies using traditional and engineered E. coli strains confirm that MaFRSA supports the biosynthesis of model proteins containing internal, aromatic, α-hydroxy acid monomers. This invention provides the first orthogonal aminoacyl-tRNA synthetase that accepts α-thio acids and α-carboxyl acids that would support carbon-carbon bond formation within the ribosome. These novel activities demonstrate the potential of PylRS as a scaffold for evolving new enzymes that act in synergy with natural or evolved ribosomes to generate diverse sequence-defined non-protein hetero-polymers.

### MaPylRS Retains Much of the Promiscuity of MmPylRS

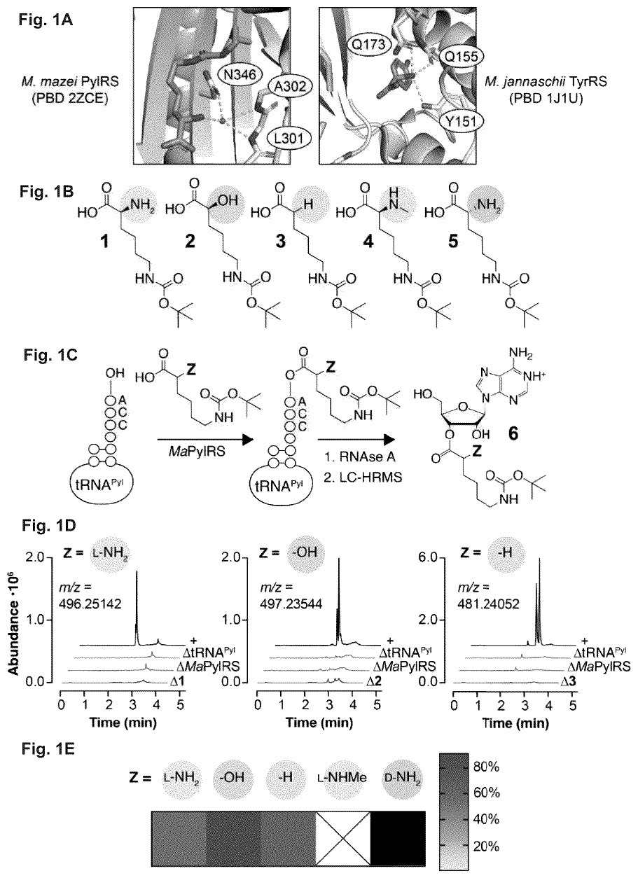

First we set out to establish whether the novel substrate scope of MmPylRS reported for L-BocK analogs (FIG. 1B)²⁰ was retained by MaPylRS, which offers advantages over MmPylRS because it lacks the poorly soluble N-terminal tRNA-binding domain³⁸ and is easier to express and evaluate in vitro³⁵. The C-terminal catalytic domain of MmPylRS³³ is 36% identical to MaPylRS and the structures are largely superimposable. To evaluate whether D-BocK as well as L-BocK analogs with —OH or —H in place of the α-amino group were substrates for MaPylRS, we made use of a validated RNAse A/LC-HRMS assay^{16,39}. This assay exploits RNAse A to cleave the phosphodiester bond of unpaired C and U residues to generate 2′, 3′-cyclic phosphate products⁴⁰. As a result, the residue at the tRNA 3′ terminus is the only mononucleoside product lacking a phosphate (FIG. 1C). Incubation of L-BocK 1 (2 mM) with purified MaPylRS (2.5 μM) and Ma-tRNA^{Pyl} (25 μM) at 37° C. for 2 hours led to a pair of RNAse A digestion products whose expected mass (496.25142 Da) corresponds to the adenosine nucleoside 6 as a mixture of 2′- and 3′-acylated species (FIG. 1D)⁴¹. No products with this mass were observed when the reaction mixture lacked Ma-tRNA^{Pyl}, L-BocK, or MaPylRS, and mass analysis of the intact tRNA product confirmed a 53.8% yield of acylated tRNA (1-tRNA^{Pyl}). Under these conditions, L-BocK analogs with either —OH (2) or —H (3) in place of the α-amino group were also substrates for MaPylRS as judged by RNAse A (FIG. 1D) and intact tRNA mass spectrometry assays with acylated tRNA yields of 90.5% (2-tRNA^{Pyl}) and 61.6% (3-tRNA^{Pyl}). No reactivity was detected with D-BocK, perhaps because of differences between PylRS from M. alvus and M. mazei⁴². We conclude that MaPylRS retains much (although not all) of the previously reported²⁰ promiscuity of MmPylRS. We note that certain non-natural monomers with relatively high activity, including α-hydroxy 2 and des-amino 3, led to measurable levels (2.5-13.7%) of diacylated tRNA (Extended Data Table 3). Diacylated tRNAs have been observed as products in cognate reactions of T. thermophilus PheRS⁴³ and are active in prokaryotic translation⁴⁴.

MaPylRS variants retain activity for phenylalanine derivatives with diverse α-amine substitutions

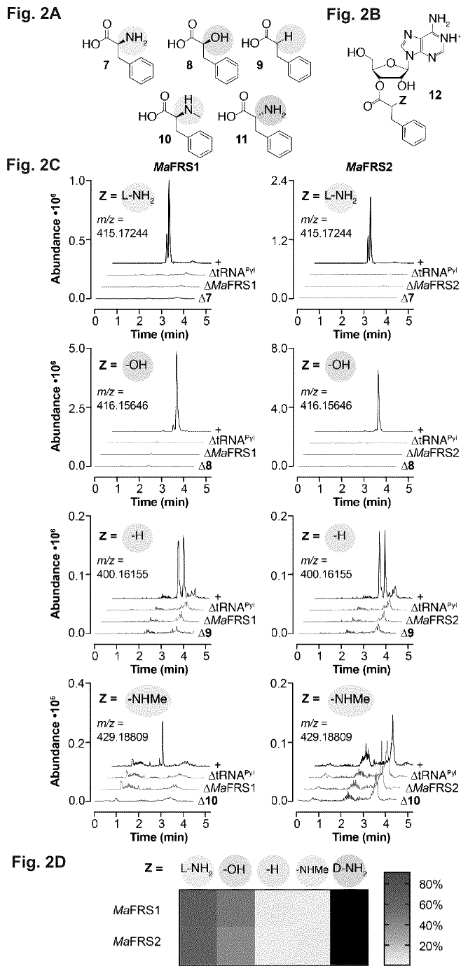

PylRS is a subclass IIc aaRS that evolved from PheRS⁴⁶. MmPylRS variants with substitutions at two positions that alter the architecture of the side chain-binding pocket (N346, C348) accept L-phenylalanine and its derivatives in place of pyrrolysine^{36,46,25}. We integrated the mutations in two variants that accept unsubstituted L-Phe (MmFRS1 and MmFRS2)³⁶ into the MaPylRS sequence to generate MaFRS1 (N166A, V168L) and MaFRS2 (N166A, V168K). We then used RNAse A and intact tRNA mass spectrometry assays to determine if MaFRS1 or MaFRS2 retained activity for L-phenylalanine 7 and analogs in which the L-α-amino group was substituted by —OH (8), —H (9), —NHCH₃ (10), or D-NH₂ (11) (FIG. 2A).

Incubation of L-Phe 7 (10 mM) with purified MaFRS1 or MaFRS2 (2.5 μM) and Ma-tRNA^{Pyl} (25 μM) at 37° C. for 2 hours led in both cases to a pair of products whose expected mass (415.17244 Da) corresponds to adenosine nucleoside 12 (Z=L-NH₂, FIG. 2B) as the expected mixture of 2′- and 3′-acylated products (FIG. 2C). No product with this mass was observed when the reaction mixture lacked Ma-tRNA^{Pyl}, L-Phe, or MaFRS1 or MaFRS2, and mass analysis of the intact tRNA product confirmed a 66.1% (MaFRS1) and 65.4% (MaFRS2) yield of acylated tRNA (7-tRNA^{Pyl}). L-Phe analogs 8-10 were all substrates for both MaFRS1 and MaFRS2 as judged by both RNAse A (FIG. 2C) and intact tRNA analysis, with reactivities in the order L-α-amino 7>α-hydroxy 8>>des-amino 9˜N-Me-L-α-amino 10 based on intact tRNA yields (FIG. 2D). Mono- and di-acylated tRNA products were observed for substrates 7 and 8 (Extended Data Table 3). Interestingly, despite the fact that the des-amino-BocK analog 3 was a strong substrate for the wild-type MaPylRS, the des-amino-Phe analog 9 had only modest activity with MaFRS1 and MaFRS2, as observed in the RNAse assay. Again, no reactivity was detected with D-Phe.

MaFRS1 & MaFRS2 Process Substrates with Novel α-Substituents

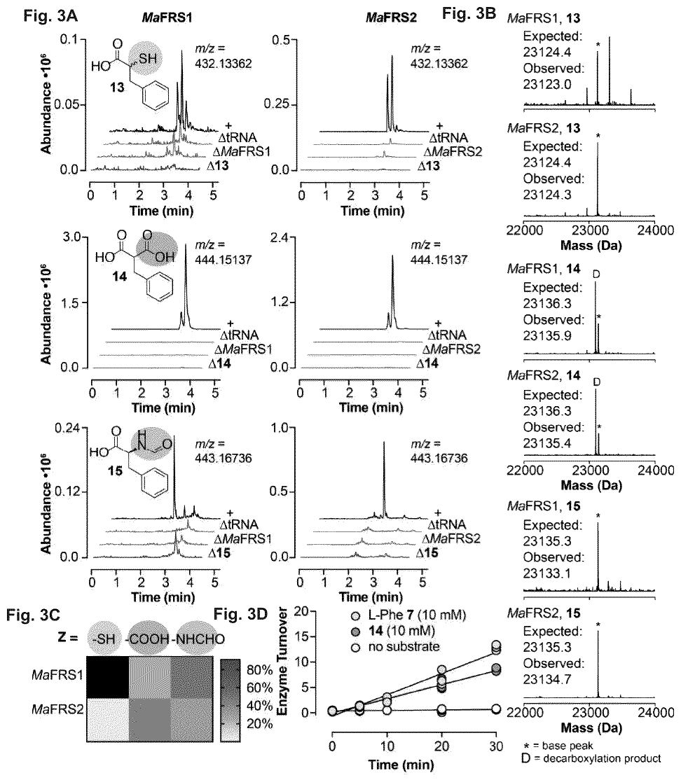

We then began to explore phenylalanine analogs with larger and electrostatically distinct functional groups at the α-carbon: α-thio acids, N-formyl-L-α-amino acids, and α-carboxyl acids (malonates) (FIG. 3A). α-thio acids are substrates for extant E. coli ribosomes in analytical-scale in vitro translation reactions with yields as high as 87% of the corresponding α-amino acids¹⁴, and thioesters can persist in E. coli of more than 36 hours⁴⁷. Peptides and proteins containing thioesters could also act as substrates for PKS modules⁴⁸ to generate unique keto-peptide natural products, or protein splicing reactions⁴⁹. Formylation of methionyl-tRNA is important for initiation complex formation, and formylation could enhance initiation with non-methionyl α-amino acids in vivo^{50,51}. Moreover, E. coli ribosomes incorporate monomers containing a 1,3-dicarbonyl moiety at the peptide N-terminus to produce keto-peptide hybrids¹⁶. To our knowledge, there are currently no aaRS enzymes, orthogonal or otherwise, that accept α-thio, N-formyl-L-α-amino, or α-carboxyl acid substrates to generate the acylated tRNAs required for in vivo translation (when extant ribosomes are compatible) or ribosome evolution (when extant ribosomes are incompatible).

We found that L-Phe analogs in which the α-amine was substituted with α-thiol (13), α-carboxyl (14), or N-formyl-L-α-amine (15) moieties were all substrates for MaFRS1 and MaFRS2 (FIG. 3A-C). In particular, α-carboxyl 14 and N-formyl-L-α-amine 15 were excellent substrates. Incubation of α-carboxyl acid 14 (10 mM) with MaFRS1 or MaFRS2 (2.5 μM) and Ma-tRNA^{Pyl} (25 μM) at 37° C. for 2 hours followed by digestion by RNAse A led to formation of a pair of products whose expected mass (444.15137 Da) corresponds to the adenosine nucleoside 12 (Z=—COOH). LC-MS analysis of intact tRNA products confirmed a 24.4% (MaFRS1) and 43.7% (MaFRS2) yield of acylated tRNA (14-tRNA^{Pyl}). Because the α-carbon of substrate 14 is prochiral, mono-acylation of Ma-tRNA^{Pyl} can generate two diastereomeric product pairs-one pair in which the 3′-hydroxyl group is acylated by the pro-S or pro-R carboxylate and another in which the 2′-hydroxyl group is acylated by the pro-S or pro-R carboxylate. These diastereomeric products result from alternative substrate orientations within the enzyme active site (vide infra).

Incubation of N-formyl-L-α-amine (15) under identical conditions led to the expected adenosine nucleoside 12 (Z=—NHCHO) and LC-MS analysis confirmed a 50.0% (MaFRS1) and 31.1% (MaFRS2) yield of acylated tRNA (15-tRNA^{Pyl}). The α-thio L-Phe analog 13 was also a substrate for MaFRS1 and MaFRS2, though higher concentrations of MaFRS1 were necessary to observe the acyl-tRNA product (13-tRNA^{Pyl}) using intact tRNA LC-MS. These results illustrate that the active site pocket that engages the α-amine in PylRS can accommodate substituents with significant differences in mass (—NHCHO) and charge (—COO⁻). Despite these differences, 2-benzylmalonate 14 (2-BMA) is an excellent substrate for MaFRS1 and MaFRS2; kinetic analysis using the malachite green assay⁵² revealed a rate of adenylation by MaFRS1 that was 66% of the rate observed for L-Phe (FIG. 3D). The tolerance for malonate substrates extends to WT MaPylRS itself: the α-carboxylate analog of L-BocK 16 was also a measurable substrate for WT MaPylRS.

### MaFRSA Processes Meta-Substituted 2-Benzylmalonic Acid Substrates and is Orthogonal to L-Phe

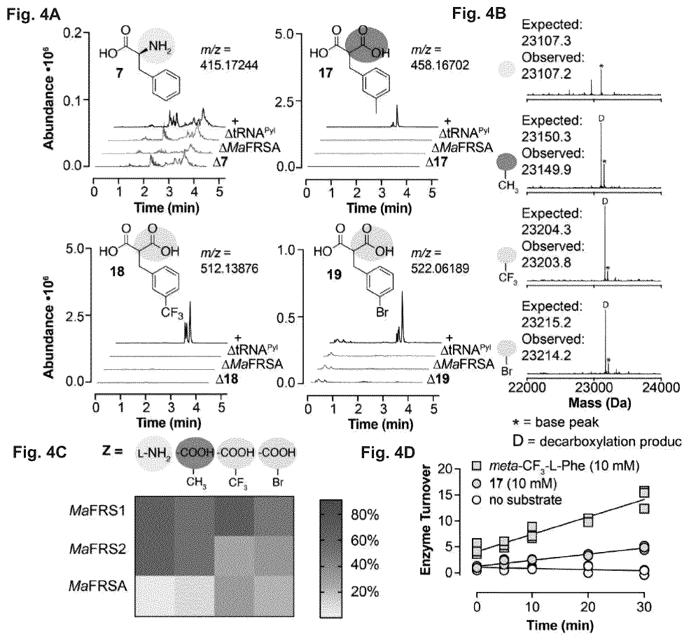

While MaFRS1 and MaFRS2 demonstrated the ability to process substrates with unusual α-substituents, they also process L-Phe with comparable efficiency (FIG. 2D), which would interfere with the selective charging of the non-L-α-amino acid. Variants of MmPylRS that accept para-, ortho-, and meta-substituted L-Phe derivatives have been reported^{36,37,53-55}. In particular, MmPylRS containing two active site mutations (N346A and C348A; henceforth referred to as FRSA) shows high activity for L-Phe analogs with bulky alkyl substituents and low activity towards L-Phe³⁷. We expressed and purified a variant of MaPylRS containing these mutations (MaFRSA: N166A, V168A) and demonstrated that it shows high activity for derivatives of malonate 14 carrying meta-CH₃ (17), meta-CF₃ (18), and meta-Br (19) substituents and low activity for L-Phe using both RNAse A (FIG. 4A) and intact tRNA analysis. Of the meta-substituted 2-benzylmalonates, MaFRSA shows the highest activity for meta-CF₃-2-benzylmalonate 18 (meta-CF₃-2-BMA). Kinetic analysis⁵² revealed a rate of adenylation that was 36% of the rate observed for the L-α-amino acid counterpart meta-CF₃-L-Phe (FIG. 4D). Although derivatives of malonate 14 carrying meta-CH₃ (17), meta-CF₃ (18), and meta-Br (19) substituents are also excellent substrates for MaFRS1 and MaFRS2, MaFRSA has significantly lower activity for L-Phe, allowing for the selective acylation of tRNA with meta-substituted 2-benzylmalonates without interference from L-Phe (FIG. 4C). Indeed, when MaFRSA is incubated with an equal concentration (10 mM) of L-Phe 7 and meta-CF₃-2-BMA 18, only the malonyl product (18-tRNA^{Pyl}) is observed.

### Structural Analysis of MaFRSA-Meta-CF₃-2-Benzylmalonate Complex Reveals Novel Interactions

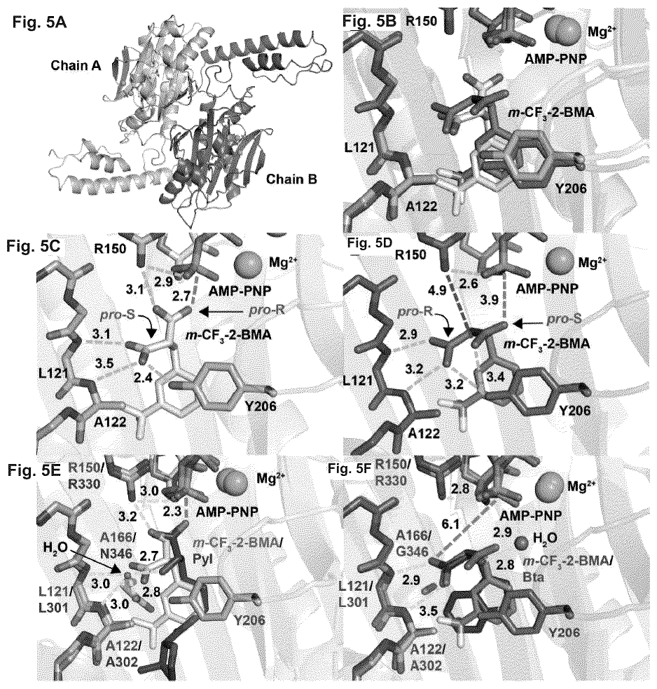

To better understand how 2-benzylmalonate substrates are accommodated by the MaFRSA active site, we solved the crystal structure of MaFRSA bound to both meta-CF₃-2-BMA and the non-hydrolyzable ATP analog adenosine 5′-(β,γ-imido)triphosphate (AMP-PNP). The structure was refined at 1.8 Å with clear substrate density for meta-CF₃-2-BMA visible at 1σ in the 2Fₒ-F_{c} map. MaFRSA crystallized with two protein chains in the asymmetric unit and an overall architecture resembling published PylRS structures (FIG. 5A)^{33,56,57}. The two protein chains in the asymmetric unit are not identical and interact with different orientations of meta-CF₃-2-BMA (FIG. 5B). One orientation of meta-CF₃-2-BMA (chain A, light purple) mimics that of L-pyrrolysine (Pyl) bound to MmPylRS³³ and would result in adenylation of the pro-R carboxylate (FIG. 5C); the other orientation (chain B, dark purple) would result in adenylation of the pro-S carboxylate (FIG. 5D).

Regardless of orientation, discrete networks of hydrogen bonds are used by MaFRSA to discriminate between the pro-R and pro-S carboxylates of meta-CF₃-2-BMA. In chain A, the pro-S carboxylate accepts a hydrogen bond from the backbone amides of L121 and A122 and the phenolic —OH of Y206. A hydrogen bond from the R150 guanidinium positions the pro-R carboxylate for nucleophilic attack with a distance of 2.7 Å between the carboxyl oxygen and the α-phosphorous of AMP-PNP. In chain B, the orientation of meta-CF₃-2-BMA is flipped relative to the conformation bound to chain A (FIG. 5D). The pro-R carboxylate accepts a hydrogen bond from the backbone amides of L121 and A122 and the phenolic —OH of Y206 as seen for the pro-S carboxylate in chain A. However, in chain B the pro-S carboxylate is rotated away from AMP-PNP and towards Y206 resulting in loss of the hydrogen bond to R150 and a longer distance of 3.9 Å between the carboxyl oxygen and the α-phosphorous of AMP-PNP. RNAse A analysis of Ma-tRNA^{Pyl} acylation by meta-CF₃-2-BMA shows more than two peaks of identical mass (FIG. 4A) that likely correspond to the two diastereomeric pairs formed from attack of the 2′- or 3′-tRNA hydroxyl group on the activated pro-R or pro-S carboxylate. More than two peaks with identical mass are also observed as RNAse A digestion products in Ma-tRNA^{Pyl} acylation reactions of other meta-substituted 2-benzylmalonates (FIG. 4A). While the meta-CF₃-2-BMA conformation in chain A appears more favorably positioned for catalysis, suggesting that the pro-R carboxylate is acylated preferentially, the appearance of more than two peaks in the RNAse A assay suggests that both conformations are catalytically competent.

As anticipated, the non-reactive, pro-S carboxylate of meta-CF₃-2-BMA is recognized by MaFRSA chain A using interactions that are distinct from those used by MmPylRS to recognize the Pyl α-amine. In the MmPylRS:Pyl:AMP-PNP complex (PDB: 2ZCE)³³, the Pyl α-amine is recognized by water-mediated hydrogen bonds to the backbone amides of L301 and A302 and the side chain carbonyl of N346, rather than by direct hydrogen bonds to the backbone amides of L121 and A122 as seen for recognition of the non-reacting carboxylate of meta-CF₃-2-BMA by both chains of MaFRSA (FIG. 5E). The interactions used to recognize the non-reacting carboxylate of meta-CF₃-2-BMA are, however, similar to those used to recognize the single carboxylate of diverse α-amino acids by other PylRS variants with mutations at N166/N346 (Ma/Mm numbering). For example, the structures of MmPylRS variants with mutations at N346, such as MmIFRS and MmBtaRS bound to 3-iodo-L-phenylalanine (3-I-F, PDB: 4TQD)⁵⁴ and 3-benzothienyl-L-alanine (Bta, PDB: 4ZIB)⁵⁸, respectively, show the substrate bound with the carboxylate directly hydrogen bonded to the L301 and A302 backbone amides, as seen for meta-CF₃-2-BMA bound to MaFRSA. In these cases, the bound water seen in the MmPylRS:Pyl:AMP-PNP complex is either absent or displaced. Mutation of N166/N346 may destabilize the water-mediated hydrogen bonding between the substrate α-amine and backbone amides seen in wild-type PylRS and promote alternative direct hydrogen bonding of a substrate carboxylate to backbone amides as seen in MaFRSA, MmIFRS, and MmBtaRS.

The largest differences between MaFRSA and other reported PylRS structures are localized to a 6-residue loop that straddles β-strands β5 and β6 and contains the active site residue Y206. In the the MmPylRS:Pyl:AMP-PNP structure (PDB: 2ZCE)³³ and the wild-type MaPylRS apo structure (PDB: 6JP2)⁵⁶, the β5-β6 or corresponding loop is either unstructured or in an open conformation positioning Y206/Y384 away from the active site, respectively. Among wild-type PylRS structures, the Y206/Y384-containing loop exists in the closed conformation only in the structure of MmPylRS bound to the reaction product, Pyl-adenylate (PDB: 2Q7H)⁵⁷. In this structure, Y384 accepts and donates a hydrogen bond to the Pyl-adenylate α-amine and pyrrole nitrogen, respectively, and forms a hydrophobic lid on the active site. In both chains of substrate-bound MaFRSA, the non-reacting carboxylate of meta-CF₃-2-BMA forms similar hydrogen bonds to Y206. These hydrogen bonds effectively close the β5-β6 loop to form a hydrophobic lid on the active site, which may contribute to the high acylation activity observed for meta-CF₃-2-BMA with MaFRSA. We note that the β5-β6 loop in chain A exhibits lower B-factors than in chain B indicating tighter binding and providing further evidence that chain A represents the more active binding mode of meta-CF₃-2-BMA. The two alternative poses of meta-CF₃-2-BMA in chains A and B correspond to a ˜120° degree rotation about the Cα-Cβ bond of the substrate that largely maintains interactions with the meta-CF₃-2-BMA side chain but alters the placement of the reacting carboxylate. This observation emphasizes the dominant stabilizing role of the main chain H-bonds provided by the backbone amides of L121 and A122.

In Vitro Translation Initiation with Novel Monomers Via Codon Skipping

We performed in vitro translation experiments to verify that the uniquely acylated derivatives of Ma-tRNA^{Pyl} produced using MaPylRS variants are effectively shuttled to and accommodated by the E. coli ribosome. Whereas the E. coli initiator tRNA^{fMet} has been engineered into a substrate for MjTyrRS variants to introduce non-canonical L-α-amino acids at the protein N-terminus⁵¹, Ma-tRNA^{Pyl} lacks the key sequence elements for recognition by E. coli initiation factors precluding its use for initiation in vivo^{35,59}. It has been reported⁶⁰ that in the absence of methionine, in vitro translation can begin at the second codon of the mRNA template, a phenomenon we refer to as “codon skipping”. We took advantage of codon skipping and a commercial in vitro translation (IVT) kit to evaluate whether Ma-tRNA^{Pyl} enzymatically charged with monomers 13-15 would support translation initiation.

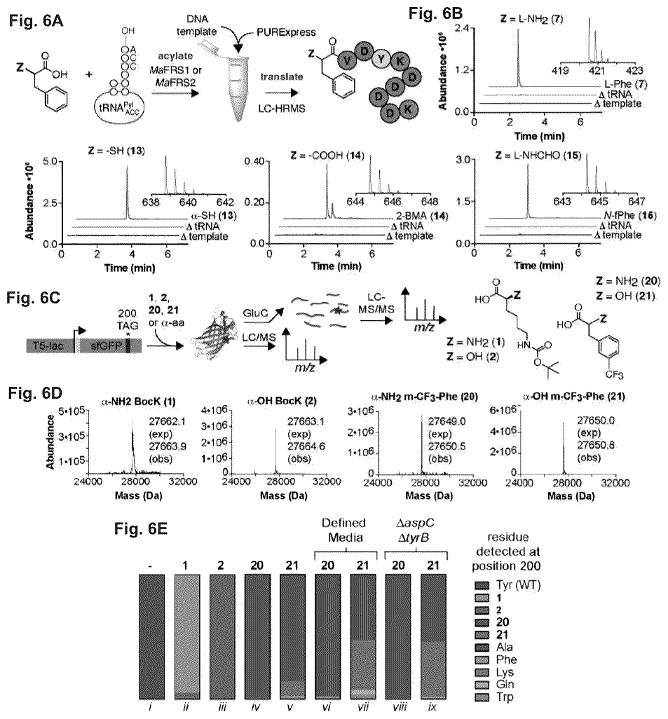

To avoid competition with release factor 1 (RF1) at UAG codons, the anticodon of Ma-tRNA^{Pyl} was mutated to ACC (Ma-tRNA^{Pyl}-ACC) to recode a glycine GGT codon at position 2 in the mRNA template. To maximize yields of acyl-tRNA, we increased the aaRS:tRNA ratio to 1:2 (monomers 7, 14, and 15) or 1:1 (monomer 13), extended the incubation time to 3 hours, and used the most active MaFRSx variant for each monomer. These modifications resulted in acyl-tRNA yields of 79% (7, MaFRS1), 13% (13, MaFRS2), 85% (14, MaFRS2), and 82% (15, MaFRS1). The acylated Ma-tRNA^{Pyl}-ACC was added with a DNA template encoding a short MGV-FLAG peptide (MGVDYKDDDDK) (SEQ ID NO:12) (FIG. 6A) to a commercial in vitro translation kit (PURExpress® Δ (aa, tRNA), NEB). When methionine was excluded from the reaction mixture, translation initiated at the second position by skipping the start codon to produce peptides with the sequence X-VDYKDDDDK (SEQ ID NO:1) (X=7, 13-15). Following FLAG-tag enrichment, LC-HRMS confirmed initiation with monomers 7 and 13-15 (FIG. 6B). When the mass corresponding to the m/z=M+2H (13-15) or m/z=M+3H (7) charge state for each peptide was extracted from the total ion chromatogram, there was a clear peak for each peptide. No such peak was observed in reactions that lacked either the DNA template or acyl-tRNA, confirming templated ribosomal initiation with α-thio acid 13, 2-benzylmalonic acid 14, and N-formyl-L-α-amino acid 15. Two peaks of identical mass are present in the EIC when translation was initiated with 2-benzylmalonic acid 14, which we assign to diastereomeric peptides resulting from acylation at either the pro-S or pro-R carboxylate. Combined with the multiple peaks present in the RNAse A assay with 2-benzylmalonic acids 17, 18, and 19, as well as the two meta-CF₃-2-BMA conformations observed in the structure of MaFRSA (FIG. 5B), the two peptide products of identical mass generated in the IVT reactions imply that MaFRSx enzymes can effectively acylate either of the two prochiral carboxylates of a malonic acid substrate.

### In Vivo Translation of Sequence-Defined Ester-Amide Hetero-Polymers

We next sought to evaluate whether the ability of MaPylRS and MaFRSA to acylate tRNA with novel monomers in vitro would support translation of backbone-modified protein heteropolymers in vivo. As the yields of tRNAs acylated in vitro with N-methyl α-amino acids and α-thio acids were low (FIG. 3), N-formyl α-amino acids lack an appropriate nucleophile, and the requirements for intra-PTC bond formation by α-carboxy (malonic) acids are unknown, we focused on the in vivo incorporation of α-OH BocK (2) and α-OH m-trifluoromethyl phenylalanine (21).

First, we modified established pUltra plasmids⁶¹ for aaRS/tRNA expression by adding a lac-operator between the tRNA promoter and coding region to make tRNA expression inducible. The resulting pMega plasmids were more easily propagated than pUltra, perhaps by alleviating toxicity⁶² from high uncharged tRNA levels during growth. Initial experiments were performed in E. coli DH10B cells transformed with one of two sfGFP reporter plasmids (pET22b-sfGFP-200TAG or pET22b-sfGFP-151TAG) and a modified pEVOL⁶³ or pMega expression plasmid encoding Ma-tRNA^{Pyl} and either MaPylRS or MaFRSA. Growths were supplemented with 1 mM BocK (1), α-OH BocK (2), m-trifluoromethyl phenylalanine (20) or α-OH m-trifluoromethyl phenylalanine (21) and the emission at 528 nm, near the λₘₐₓ for sfGFP, was assessed after 24 h. Under these conditions, sfGFP production relies on MaPylRS or a variant thereof to charge Ma-tRNA^{Pyl} with an α-OH or α-NH₂ acid provided in the growth media followed by ribosomal elongation of the charged monomer. In most cases, DH10B cells harboring a pMega plasmid produced 2-3 fold higher levels of sfGFP fluorescence than those harboring a pEVOL plasmid. In all but one case, α-OH monomers led to approximately 1.5-2-fold lower sfGFP fluorescence than α-NH₂ monomers. The highest levels of sfGFP fluorescence were observed in cases in which α-NH₂ or α-OH monomers were encoded at position 200.

Preparative scale growths were conducted using pET22b-sfGFP-200TAG and pMega-MaPylRS or pMega-MaFRSA, the top-performing plasmids in the plate reader assay, and the sfGFP products were isolated by metal-affinity chromatography. As observed previously with WT MmPylRS, E. coli DH10B cells expressing MaPylRS and grown in the presence of 1 mM BocK (1) or α-OH BocK (2) expressed an sfGFP variant whose mass corresponded to the presence of a single BocK side chain (FIG. 6D); analogous cells expressing MaFRSA and grown in the presence of 1 mM m-trifluoromethyl phenylalanine (20) or α-OH m-trifluoromethyl phenylalanine (21) expressed an sfGFP variant whose mass corresponded to the presence of a single trifluoromethyl phenylalanine side chain (FIG. 6D). Although the intact masses of proteins grown in the presence of BocK (1) and m-trifluoromethyl phenylalanine (20) differed from those grown in the presence of α-OH BocK (2) or α-OH m-trifluoromethyl phenylalanine (21), the resolution was insufficient to unequivocally establish the ester:amide ratio. To more rigorously characterize the products, we digested the isolated sfGFP variants with GluC and analyzed the products by LC-MS/MS (FIG. 6E, columns i through v. This analysis revealed that sfGFP produced in the presence of α-OH BocK (2) contained virtually only an ester linkage at position 200, but that the sfGFP product generated in the presence of α-OH m-trifluoromethyl phenylalanine (21) contained an ester:amide ratio of 11:85 (FIG. 6E, Extended Data Table 5).

Certain α-hydroxy acids can be metabolized in E. coli into α-amino acids via a two step oxidation/trans-amination process. Indeed, in classic work¹⁹, a DH10B strain lacking the transaminases aspC and tyrB was required to detect cytosolic accumulation of the α-OH analog of tyrosine, 4-hydroxyphenyl lactic acid¹⁹. We thus repeated preparative scale growths of DH10B ΔaspC ΔtyrB harboring pET22b-sfGFP-200TAG and pMega-MaFRSA supplemented with 1 mM of m-trifluoromethyl phenylalanine (20) or α-OH m-trifluoromethyl phenylalanine (21). For comparison, we also performed growths in DH10B but using a defined media lacking glutamate, the amine donor for aspC and tyrB, in an attempt to minimize transaminase activity. Intact mass analysis of sfGFP isolated from DH10B ΔaspC ΔtyrB growth supplemented with α-OH m-trifluoromethyl phenylalanine (21) were again consistent with the expected products. Both strategies improved the fraction of ester product as judged by GluC digestion of intact isolated sfGFP; use of defined media led to a 39:52 ester:amide ratio whereas use of DH10B ΔaspC ΔtyrB led to an ester:amide ratio of 44:52. These studies reveal that while MaFRSA is sufficiently active in vivo to support the biosynthesis of a sequence-defined hetero-oligomer, more complex E. coli strains or alternative organisms⁶⁴ may be required to generate ribosomal products containing multiple esters in high yield.

### Discussion

Expanding and reprogramming the genetic code for the templated biosynthesis of sequence-defined hetero-polymers demands orthogonal aminoacyl-tRNA synthetase/tRNA pairs that accept non-L-α-amino acid substrates. Fahnestock & Rich reported over fifty years ago that the peptidyl transferase center (PTC) of the E. coli ribosome tolerates an α-hydroxyl substituent in place of the α-amine of phenylalanine and described the in vitro biosynthesis of a polyester³. More recent in vitro studies have broadened the scope of wild-type PTC reactivity to include diverse nucleophilic heteroatoms in place of the α-amine¹³⁻¹⁶. However, with the exception of substrates carrying an α-hydroxyl substituent^{19-21,65}, none of these non-L-α-amino acid monomers are substrates for any known orthogonal aminoacyl-tRNA synthetase. The challenge is that most aminoacyl-tRNA synthetases of known structure simultaneously engage both the substrate α-amine and α-carboxylate moieties via multiple main-chain and side-chain hydrogen bonds to position the α-carboxylate for adenylation and acylation. These well-conserved hydrogen bond networks complicate the engineering of new enzymes that accept substrates with alternative amine/carboxylate orientations or conformations (such as β-amino acids or aramids), or those whose α-substituents are large and/or electrostatically distinctive (such as malonates, α-thio acids, and N-acyl α-amino acids).

Yokoyama and coworkers demonstrated that the water-mediated hydrogen-bond network in the active site of Methanosarcina mazei pyrrolysyl-tRNA synthetase facilitated recognition of substrates with conservative substitutions (α-OH, α-H, α-NHCH₃) of the α-amino group²⁰. Here we expand the scope of monomers recognized and processed by PylRS variants to include α-thio acids and N-formyl-L-α-amino acids as well as those that carry an α-carboxyl functional group in place of the α-amine: prochiral malonic acids with protein-like side chains. Monomers containing α-thio, N-formyl-L-α-amino and α-carboxyl substituents in place of the α-amine can be incorporated into polypeptides at the N-terminus by the native E. coli translational apparatus; those with an α-hydroxy substitute can be introduced into proteins in vivo, albeit in a side-chain and position-specific manner. Biopolymers produced at scale containing multiple, distinct ester units can serve as the basis for biomaterials that change shape and self-cleave in a pH and/or environment-selective manner.

Although thioesters and malonic acids are ubiquitous intermediates in polyketide and fatty acid biosynthesis^{66,67}, as far as we know, aaRS enzymes that act on α-thio or α-carboxyl acids are unknown and tRNAs acylated with a polyketide precursor represent novel chemical species. Such tRNAs can forge a new link between ribosomal translation and assembly-line polyketide synthases⁶⁸, the molecular machines responsible for protein and polyketide biosynthesis, respectively. Combined with synthetic genomes^{69,70}, ribosomes capable of carbon-carbon bond formation enables template-driven biosynthesis of unique hybrid biomaterials and sequence-defined polyketide-peptide oligomers, such as those produced by PKS-NRPS biosynthetic modules.

### Methods

Expression and purification of MaPylRS, MaFRS1, MaFRS2, and MaFRSA for biochemistry. Plasmids used to express wild-type (WT) MaPylRS (pET32a-MaPylRS) and MaFRS1 (pET32a-MaFRS1) were constructed by inserting synthetic dsDNA fragments (Extended Data Table 1) into the NdeI-NdeI cut sites of a pET32a vector using the Gibson method⁷¹. pET32a-MaFRS2 and pET32a-MaFRSA were constructed from pET32a-MaFRS1 using a Q5® Site-Directed Mutagenesis Kit (NEB). Primers RF31 & RF32, and RF32 & RF33 (Extended Data Table 1) were used to construct pET32a-MaFRS2 and pET32a-MaFRSA, respectively. The sequences of the plasmids spanning the inserted regions were confirmed via Sanger sequencing at the UC Berkeley DNA Sequencing Facility using primers T7 F and T7 R (Extended Data Table 1) and the complete sequence of each plasmid was confirmed by the Massachusetts General Hospital CCIB DNA Core.

Chemically competent cells were prepared by following a modified published protocol⁷². Briefly, 5 mL of LB was inoculated using a freezer stock of BL21-Gold (DE3)pLysS cells. The following day, 50 mL of LB was inoculated with 0.5 mL of the culture from the previous day and incubated at 37° C. with shaking at 200 rpm until the culture reached an OD₆₀₀ between 0.3-0.4. The cells were collected by centrifugation at 4303×g for 20 min at 4° C. The cell pellet was resuspended in 5 mL of sterile filtered TSS solution (10% w/v polyethylene glycol 8000, 30 mM MgCl₂, 5% v/v DMSO in 25 g/L LB). The chemically competent cells were portioned into 100 μL aliquots in 1.5 mL microcentrifuge tubes, flash frozen in liquid N₂, and stored at −80° C. until use. The following protocol was used to transform plasmids into chemically competent cells: 20 μL of KCM solution (500 mM KCl, 150 mM CaCl₂, 250 mM MgCl₂) was added to a 100 μL aliquot of cells on ice along with approximately 200 ng of the requisite plasmid and water to a final volume of 200 μL. The cells were incubated on ice for 30 min and then heat-shocked by placing them for 90 s in a water-bath heated to 42° C. Immediately after heat shock the cells were placed on ice for 2 min, after which 800 μL of LB was added. The cells then incubated at 37° C. with shaking at 200 rpm for 60 min. The cells were plated onto LB-agar plates with the appropriate antibiotic and incubated overnight at 37° C.

Plasmids used to express wild type (WT) MaPylRS, MaFRS1, MaFRS2 and MaFRSA were transformed into BL21-Gold (DE3)pLysS chemically competent cells and plated onto LB agar plates supplemented with 100 μg/mL carbenicillin. Colonies were picked the following day and used to inoculate 10 mL of LB supplemented with 100 μg/mL carbenicillin. The cultures were incubated overnight at 37° C. with shaking at 200 rpm. The following day the 10 mL cultures were used to inoculate 1 L of LB supplemented with 100 μg/mL carbenicillin in 4 L baffled Erlenmeyer flasks. Cultures were incubated at 37° C. with shaking at 200 rpm for 3 h until they reached an OD₆₀₀ of 0.6-0.8. At this point, isopropyl β-D-1-thiogalactopyranoside (IPTG) was added to a final concentration of 1 mM and incubation was continued for 6 h at 30° C. with shaking at 200 rpm. Cells were harvested by centrifugation at 4303×g for 20 min at 4° C. and the cell pellets were stored at −80° C. until the expressed protein was purified as described below.

The following buffers were used for protein purification: Wash buffer: 50 mM sodium phosphate (pH 7.4), 500 mM NaCl, 20 mM β-mercaptoethanol (BME), 25 mM imidazole; Elution buffer: 50 mM sodium phosphate (pH 7.4), 500 mM NaCl, 20 mM β-mercaptoethanol (BME), 100 mM imidazole; Storage buffer: 100 mM HEPES-K, pH 7.2, 100 mM NaCl, 10 mM MgCl₂, 4 mM dithiothreitol (DTT), 20% v/v glycerol. 1 cOmplete Mini EDTA-free protease inhibitor tablet was added to Wash and Elution buffers immediately before use. To isolate protein, cell pellets were resuspended in Wash buffer (5 mL/g cells). The resultant cell paste was lysed at 4° C. by sonication (Branson Sonifier 250) over 10 cycles consisting of 30 sec sonication followed by 30 sec manual swirling. The lysate was centrifuged at 4303×g for 10 min at 4° C. to separate the soluble and insoluble fractions. The soluble lysate was incubated at 4° C. with 1 mL of Ni-NTA agarose resin (washed with water and equilibrated with Wash buffer) for 2 h. The lysate-resin mixture was added to a 65 g RediSep® Disposable Sample Load Cartridge (Teledyne ISCO) and allowed to drain at RT. The protein-bound Ni-NTA agarose resin was then washed with three 10 mL aliquots of Wash buffer. The protein was eluted from Ni-NTA agarose resin by rinsing the resin three times with 10 mL Elution buffer. The elution fractions were pooled and concentrated using a 10 kDa MWCO Amicon® Ultra-15 Centrifugal Filter Unit (4303×g, 4° C.). The protein was then buffer-exchanged into Storage buffer until the [imidazole] was <5 μM using the same centrifugal filter unit. The protein was dispensed into 20 μL single-use aliquots and stored at −80° C. for up to 8 months. Protein concentration was measured using the Bradford assay⁷³. Yields were between 8 and 12 mg/L. Proteins were analyzed by SDS-PAGE using Any kD™ Mini-PROTEAN® TGX™ Precast Protein Gels (BioRad). The gels were run at 200 V for 30 min.

Proteins were analyzed by LC-MS to confirm their identities. Samples analyzed by mass spectrometry were resolved using a Poroshell StableBond 300 C8 (2.1×75 mm, 5 μm, Agilent Technologies part #660750-906) using a 1290 Infinity II UHPLC (G7120AR, Agilent). The mobile phases used for separation were (A) 0.1% formic acid in water and (B) 100% acetonitrile, and the flow rate was 0.4 mL/min. After an initial hold at 5% (B) for 0.5 min, proteins were eluted using a linear gradient from 5 to 75% (B) for 9.5 min, a linear gradient from 75 to 100% (B) for 1 min, a hold at 100% (B) for 1 min, a linear gradient 100 to 5% (B) for 3.5 min, and finally a hold at 5% (B) for 4.5 min. Protein masses were analyzed using LC-HRMS with an Agilent 6530 Q-TOF AJS-ESI (G6530BAR). The following parameters were used: gas temperature 300° C., drying gas flow 12 L/min, nebulizer pressure 35 psi, sheath gas temperature 350° C., sheath gas flow 11 L/min, fragmentor voltage 175 V, skimmer voltage 65 V, Oct 1 RF Vpp 750 V, Vcap 3500 V, nozzle voltage 1000 V, 3 spectra/s.

Analytical size exclusion chromatography was performed on an ÄKTA Pure 25. A flow rate of 0.5 mL/min was used for all steps. A Superdex® 75 Increase 10/300 GL column (stored and operated at 4° C.) was washed with 1.5 column volumes (CV) of degassed and sterile filtered MilliQ water. The column equilibrated in 1.5 column volumes of SEC Buffer: 100 mM HEPES (pH 7.2), 100 mM NaCl, 10 mM MgCl₂, 4 mM DTI. Approximately 800 μg of protein in 250 μL SEC Buffer was loaded into a 500 μL capillary loop. The sample loop was washed with 2.0 mL of SEC Buffer as the sample was injected onto the column. The sample was eluted in 1.5 column volumes of SEC Buffer and analyzed by UV absorbance at 280 nm.

Transcription and purification of tRNAs. The DNA template used for transcribing M. alvus tRNA^{Pyl} (Ma-tRNA^{Pyl})³⁵ was prepared by annealing and extending the ssDNA oligonucleotides Ma-PylT-F and Ma-PylT-R (2 mM, Extended Data Table 1) using OneTaq 2× Master Mix (NEB). The annealing and extension used the following protocol on a thermocycler (BioRad C1000 Touch™): 94° C. for 30 s, 30 cycles of [94° C. for 20 s, 53° C. for 30 s, 68° C. for 60 s], 68° C. for 300 s. Following extension, the reaction mixture was supplemented with sodium acetate (pH 5.2) to a final concentration of 300 mM, washed once with 1:1 (v/v) acid phenol:chloroform, twice with chloroform, and the dsDNA product precipitated upon addition of ethanol to a final concentration of 71%. The pellet was resuspended in water and the concentration of dsDNA determined using a NanoDrop ND-1000 (Thermo Scientific). The template begins with a single C preceding the T7 promoter, which increases yields of T7 transcripts⁷⁴. The penultimate residue of Ma-PylT-R carries a 2′-methoxy modification, which reduces non-templated nucleotide addition by T7 RNA polymerase during in vitro transcription⁷⁵.

Ma-tRNA^{Pyl} was transcribed in vitro using a modified version of a published procedure⁷⁶. Transcription reactions (25 μL) contained the following components: 40 mM Tris-HCl (pH 8.0), 100 mM NaCl, 20 mM DTT, 2 mM spermidine, 5 mM adenosine triphosphate (ATP), 5 mM cytidine triphosphate (CTP), 5 mM guanosine triphosphate (GTP), 5 mM uridine triphosphate (UTP), 20 mM guanosine monophosphate (GMP), 0.2 mg/mL bovine serum albumin, 20 mM MgCl₂, 12.5 ng/μL DNA template, 0.025 mg/mL T7 RNA polymerase. These reactions were incubated at 37° C. in a thermocycler for 3 h. Four 25 μL reactions were pooled, and sodium acetate (pH 5.2) was added to a final concentration of 300 mM in a volume of 200 μL. The transcription reactions were extracted once with 1:1 (v/v) acid phenol:chloroform, washed twice with chloroform, and the tRNA product precipitated by adding ethanol to a final concentration of 71%. After precipitation, the tRNA pellet was resuspended in water and incubated with 8 U of RQ1 RNAse-free DNAse (Promega) at 37° C. for 30 min according to the manufacturer's protocol. The tRNA was then washed with phenol:chloroform and chloroform as described above, precipitated, and resuspended in water. To remove small molecules, the tRNA was further purified using a Micro Bio-Spin™ P-30 Gel Column, Tris Buffer RNase-free (Bio-Rad) after first exchanging the column buffer to water according to the manufacturer's protocol. The tRNA was precipitated once more, resuspended in water, quantified using a NanoDrop ND-1000, aliquoted, and stored at −20° C.

tRNA was analyzed by Urea-PAGE using a 10% Mini-PROTEAN® TBE-Urea Gel (BioRad). The gels were run at 120 V for 30 min then stained with SYBR-Safe gel stain (Thermo-Fisher) for 5 minutes before imaging. Ma-tRNA^{Pyl} was analyzed by LC-MS to confirm its identity. Samples were resolved on a ACQUITY UPLC BEH C18 Column (130 Å, 1.7 μm, 2.1 mm×50 mm, Waters part #186002350, 60° C.) using an ACQUITY UPLC I-Class PLUS (Waters part #186015082). The mobile phases used were (A) 8 mM triethylamine (TEA), 80 mM hexafluoroisopropanol (HFIP), 5 μM ethylenediaminetetraacetic acid (EDTA, free acid) in 100% MilliQ water; and (B) 4 mM TEA, 40 mM HFIP, 5 μM EDTA (free acid) in 50% MilliQ water/50% methanol. The method used a flow rate of 0.3 mL/min and began with Mobile Phase B at 22% that increased linearly to 40% B over 10 min, followed by a linear gradient from 40 to 60% B for 1 min, a hold at 60% B for 1 min, a linear gradient from 60 to 22% B over 0.1 min, then a hold at 22% B for 2.9 min. The mass of the RNA was analyzed using LC-HRMS with a Waters Xevo G2-XS Tof (Waters part #186010532) in negative ion mode with the following parameters: capillary voltage: 2000 V, sampling cone: 40, source offset: 40, source temperature: 140° C., desolvation temperature: 20° C., cone gas flow: 10 L/h, desolvation gas flow: 800 L/h, 1 spectrum/s. Expected masses of oligonucleotide products were calculated using the AAT Bioquest RNA Molecular Weight Calculator⁷⁷. Deconvoluted mass spectra were obtained using the MaxEnt software (Waters Corporation).

Procedure for RNAse A assays. Reaction mixtures (25 μL) used to acylate tRNA contained the following components: 100 mM Hepes-K (pH 7.5), 4 mM DTT, 10 mM MgCl₂, 10 mM ATP, 0-10 mM substrate, 0.1 U E. coli inorganic pyrophosphatase (NEB), 25 μM Ma-tRNA^{Pyl}, and 2.5 μM enzyme (MaPylRS, MaFRS1, MaFRS2, or MaFRSA). Reaction mixtures were incubated at 37° C. in a dry-air incubator for 2 h. tRNA samples from enzymatic acylation reactions were quenched with 27.5 μL of RNAse A solution (1.5 U/μL RNAse A (MilliporeSigma), 200 mM sodium acetate, pH 5.2) and incubated for 5 min at room temperature. Proteins were then precipitated upon addition of 50% trichloroacetic acid (TCA, Sigma-Aldrich) to a final concentration of 5%. After precipitating protein at −80° C. for 30 min, insoluble material was removed by centrifugation at 21,300×g for 10 min at 4° C. The soluble fraction was then transferred to autosampler vials, kept on ice until immediately before LC-MS analysis, and returned to ice immediately afterwards.

Samples analyzed by mass spectrometry were resolved using a Zorbax Eclipse XDB-C18 RRHD column (2.1×50 mm, 1.8 μm, room temperature, Agilent Technologies part #981757-902) fitted with a guard column (Zorbax Eclipse XDB-C18, 2.1×5 mm 1.8 μm, Agilent part #821725-903) using a 1290 Infinity II UHPLC (G7120AR, Agilent). The mobile phases used were (A) 0.1% formic acid in water; and (B) 100% acetonitrile. The method used a flow rate of 0.7 mL/min and began with Mobile Phase B held at 4% for 1.35 min, followed by a linear gradient from 4 to 40% B over 1.25 min, a linear gradient from 40 to 100% B over 0.4 min, a linear gradient from 100 to 4% B over 0.7 min, then finally B held at 4% for 0.8 min. Acylation was confirmed by correctly identifying the exact mass of the 2′ and 3′ acyl-adenosine product corresponding to the substrate tested in the extracted ion chromatogram by LC-HRMS with an Agilent 6530 Q-TOF AJS-ESI (G6530BAR). The following parameters were used: fragmentor voltage of 175 V, gas temperature of 300° C., gas flow of 12 L/min, sheath gas temperature of 350° C., sheath gas flow of 12 L/min, nebulizer pressure of 35 psi, skimmer voltage of 65 V, Vcap of 3500 V, and collection rate of 3 spectra/s. Expected exact masses of acyl-adenosine nucleosides (Extended Data Table 2) were calculated using ChemDraw 19.0 and extracted from the total ion chromatograms ±100 ppm.

Procedure for determining aminoacylation yields using intact tRNA mass spectrometry. Enzymatic tRNA acylation reactions (25 μL) were performed as described in Procedure for RNAse A assays. Sodium acetate (pH 5.2) was added to the acylation reactions to a final concentration of 300 mM in a volume of 200 μL. The reactions were then extracted once with a 1:1 (v/v) mixture of acidic phenol (pH 4.5):chloroform and washed twice with chloroform. After extraction, the acylated tRNA was precipitated by adding ethanol to a final concentration of 71% and incubation at −80° C. for 30 min, followed by centrifugation at 21,300×g for 30 min at 4° C. After the supernatant was removed, acylated tRNA was resuspended in water and kept on ice for analysis.

tRNA samples from enzymatic acylation reactions were analyzed by LC-MS as described in Transcription and purification of tRNAs. Because the unacylated tRNA peak in each total ion chromatogram (TIC) contains tRNA species that cannot be enzymatically acylated (primarily tRNAs that lack the 3′ terminal adenosine⁷⁸), simple integration of the acylated and non-acylated peaks in the A₂₆₀ chromatogram does not accurately quantify the acylation yield. To accurately quantify acylation yield, we used the following procedure. For each sample, the mass data was collected between 500 and 2000 m/z. A subset of the mass data collected defined as the raw MS deconvolution range was used to produce the deconvoluted mass spectra. The raw MS deconvolution range of each macromolecule species contains multiple peaks that correspond to different charge states of that macromolecule. Within the raw mass spectrum deconvolution range we identified the most abundant charge state peak in the raw mass spectrum of each tRNA species (unacylated tRNA, monoacylated tRNA, and diacylated tRNA). To quantify the relative abundance of each species, the exact mass of the major ions ±0.3000 Da was extracted from the TIC to produce extracted ion chromatograms (EICs). The EICs were integrated and the areas of the peaks that aligned with the correct peaks in the TIC (as determined from the deconvoluted mass spectrum) were used for quantification of yields (Extended Data Table 3). For malonic acid substrates, the integrated peak areas for the EICs from both the malonic acid product and the decarboxylation product are added together to determine the overall acylation yield. Each sample was injected 3 times; chromatograms and spectra are representative, yields shown in Extended Data Table 3 are an average of the 3 injections. Expected masses of oligonucleotide products were calculated using the AAT Bioquest RNA Molecular Weight Calculator⁷⁷ and the molecular weights of the small molecules added to them were calculated using ChemDraw 19.0. All masses identified in the mass spectra are summarized in Extended Data Data.

Malachite green assay to monitor adenylation. Enzymatic adenylation reactions were monitored using malachite green using a previous protocol with modifications⁵². Each adenylation reaction (60 μL) contained the following components: 200 mM HEPES-K (pH 7.5), 4 mM DTT, 10 mM MgCl₂, 0.2 mM ATP, 0-10 mM substrate, 4 U/mL E. coli inorganic pyrophosphatase (NEB), and 2.5 μM enzyme (MaFRS1 or MaFRSA). Adenylation reactions were incubated at 37° C. in a dry-air incubator. Aliquots (10 μL) were withdrawn after 0, 5, 10, 20, and 30 min and quenched upon addition to an equal volume of 20 mM EDTA (pH 8.0) on ice. Once all aliquots were withdrawn, 80 μL of Malachite Green Solution (Echelon Biosciences) was added to each aliquot and the mixture incubated at RT for 30 min. After shaking for 30 sec to remove bubbles, the absorbance at 620 nm was measured on a Synergy HTX plate reader (BioTek). The absorbance was then converted to phosphate concentration using a phosphate standard curve (0-100 μM) and plotted over time to determine turnover numbers.

Structure determination. The following synthetic dsDNA sequence was cloned upstream of MaFRSA (MaPylRS N166A:V168A) into pET32a-MaFRSA by Gibson assembly⁷¹ and used for subsequent crystallographic studies: GSS linker-6×His-SSG linker-thrombin site-MaFRSA (Extended Data Table 1). The sequence of the pET32a-6×His-thrombin-MaFRSA plasmid was confirmed with Sanger sequencing from Genewiz using primers T7 F and T7 R (Extended Data Table 1). The procedure used to express and purify MaFRSA for crystallography using pET32a-6×His-thrombin-MaFRSA was adapted from a reported protocol used to express and purify wild-type M. alvus PylRS by Seki et al.⁵⁶. BL21(DE3) Gold competent cells (Agilent Technologies) were transformed with pET32a-6×His-thrombin-MaFRSA and grown in TB media at 37° C. Protein expression was induced at an OD₆₀₀ reading of 1.2 with 1 mM isopropyl β-D-1-thiogalactopyranoside (IPTG). The temperature was lowered to 20° C. and growth continued overnight. Cells were pelleted for 1 h at 4,300×g and resuspended in Lysis Buffer (50 mM potassium phosphate (pH 7.4), 25 mM imidazole, 500 mM sodium chloride, 5 mM β-mercaptoethanol, 1 cOmplete Mini EDTA-free protease inhibitor tablet). Cells were lysed by homogenization (Avestin Emulsiflex C3). After centrifugation for 1 h at 10,000×g, the clarified lysate was bound to TALON® Metal Affinity Resin (Takara Bio) for 1 h at 4° C., washed with additional lysis buffer, and eluted with Elution Buffer (50 mM potassium phosphate (pH 7.4), 500 mM imidazole, 500 mM sodium chloride, 5 mM β-mercaptoethanol). The eluate was dialyzed overnight at 4° C. into Cleavage Buffer (40 mM potassium phosphate (pH 7.4), 100 mM NaCl, 1 mM dithiothreitol (DTT)) then incubated overnight at room temperature with thrombin protease on a solid agarose support (MilliporeSigma). Following cleavage, the protein was passed over additional TALON® resin to remove the 6×His tag and dialyzed overnight at 4° C. into Sizing buffer (30 mM potassium phosphate (pH 7.4), 200 mM NaCl, 1 mM DTT). The protein was concentrated and loaded onto a HiLoad® 16/600 Superdex® 200 pg column (Cytiva Life Sciences) equilibrated with Sizing buffer on an ÄKTA Pure 25 fast-liquid chromatography machine. Purified MaFRSA was dialyzed into Storage Buffer (10 mM Tris-HCl (pH 8.0), 150 mM NaCl, 10 mM MgCl2, 10 mM β-mercaptoethanol), concentrated to 20 mg/mL, aliquoted, and flash-frozen for crystallography.

Initial crystallization screening conditions were adapted from Seki et al.⁵⁶. Crystals were grown by hanging drop vapor-diffusion in 24-well plates. 25 μL of 100 mM meta-trifluoromethyl-2-benzylmalonate (meta-CF₃-2-BMA) pH ˜7 was added to 1.5 mL microcentrifuge tubes and the water was removed by evaporation. The dried aliquots were then resuspended at a concentration of 100 mM with MaFRSA in Storage Buffer at three concentrations (6.9, 12.3, and 19.2 mg/mL) and 10 mM adenosine 5′-(β,γ-imido)triphosphate lithium salt hydrate (AMP-PNP). The protein/substrate solution (1 μL) was mixed in a 1:1 ratio with the reservoir solution (1 μL) containing 10 mM Tris-HCl pH 7.4 and 26% polyethylene glycol 3350 and incubated over 1 mL of reservoir solution at 18° C. Crystals with an octahedral shape appeared within one week. Crystals were plunged into liquid nitrogen to freeze with no cryoprotectant.

Data were collected at the Advanced Light Source beamline 8.3.1 at 100 K with a wavelength of 1.11583 Å. Data collection and refinement statistics are presented in Extended Data Table 4. Diffraction data were indexed and integrated with XDS⁷⁹, then merged and scaled with Pointless⁸⁰ and Aimless⁸¹. The crystals were in the space group I4, and the unit cell dimensions were 108.958, 108.958, and 112.26 Å. The structure was solved by molecular replacement with Phaser⁸² using a single chain of the wild-type apo structure of M. alvus PylRS (PDB code: 6JP2)⁵⁶ as the search model. There were two copies of MaFRSA in the asymmetric unit. The model was improved with iterative cycles of manual model building in COOT⁸³ alternating with refinement in Phenix^{84,85} using data up to 1.8 Å resolution. Structural analysis and figures were generated using Pymol version 2.4.2⁸⁶.

In vitro translation initiation. The Ma-tRNA^{Pyl}-ACC dsDNA template was prepared as described in Transcription and purification of tRNAs using the primers Ma-PylT-ACC F and Ma-PylT-ACC R (Extended Data Table 1). Ma-tRNA^{Pyl}-ACC was also transcribed, purified, and analyzed as described previously. Enzymatic tRNA acylation reactions (150 μL) were performed as described in Procedure for RNAse A assays with slight modifications. The enzyme concentration was increased to 12.5 μM (monomers 7, 14, and 15) or 25 μM (monomer 13) and the incubation time was increased to 3 hours at 37° C. Sodium acetate (pH 5.2) was added to the acylation reactions to a final concentration of 300 mM in a volume of 200 μL. The reactions were then extracted once with a 1:1 (v/v) mixture of acidic phenol (pH 4.5):chloroform and washed twice with chloroform. After extraction, the acylated tRNA was precipitated by adding ethanol to a final concentration of 71% and incubation at −80° C. for 30 min followed by centrifugation at 21,300×g for 30 min at 4° C. Acylated tRNAs were resuspended in water to a concentration of 307 μM immediately before in vitro translation.

Templates for expression of MGVDYKDDDDK (SEQ ID NO:12) were prepared by annealing and extending the oligonucleotides MGVflag-1 and MGVflag-2 using Q5® High-Fidelity 2× Master Mix (NEB) (Extended Data Table 1). The annealing and extension used the following protocol on a thermocycler (BioRad C1000 Touch™): 98° C. for 30 s, 10 cycles of [98° C. for 10 s, 55° C. for 30 s, 72° C. for 45 s], 10 cycles of [98° C. for 10 s, 67° C. for 30 s, 72° C. for 45 s], and 72° C. for 300 s. Following extension, the reaction mixture was supplemented with sodium acetate (pH 5.2) to a final concentration of 300 mM, extracted once with a 1:1 (v/v) mixture of basic phenol (pH 8.0):chloroform, and washed twice with chloroform. The dsDNA product was precipitated upon addition of ethanol to a final concentration of 71% and incubation at −80° C. for 30 min followed by centrifugation at 21,300×g for 30 min at 4° C. The dsDNA pellets were washed once with 70% (v/v) ethanol and resuspended in 10 mM Tris-HCl pH 8.0 to a concentration of 500 ng/μL and stored at −20° C. until use in translation.

In vitro transcription/translation by codon skipping of the short FLAG tag-containing peptides X-Val-Asp-Tyr-Lys-Asp-Asp-Asp-Asp-Lys (XV-Flag) (SEQ ID NO:1) where X=7, 13, 14, or 15 was carried out using the PURExpress® Δ (aa, tRNA) Kit (New England Biolabs, E6840S) based on a previous protocol with slight modifications¹⁶. The XV-Flag peptides were produced with the following reactions (12.5 μL): Solution A (ΔtRNA, Δaa; 2.5 μL), amino acid stock mix (1.25 μL; 33 mM L-valine, 33 mM L-aspartic acid, 33 mM L-tyrosine, 33 mM L-lysine), tRNA solution (1.25 μL), Solution B (3.75 μL), 250 ng dsDNA MGVDYKDDDDK (SEQ ID NO:12) template (0.5 μL), and Ma-tRNA^{Pyl}-ACC acylated with 7, 13, 14, or 15 (3.25 μL). The reactions were incubated in a thermocycler (BioRad C1000 Touch™) at 37° C. for 2 hours and quenched by placement on ice.

Translated peptides were purified from in vitro translation reactions by enrichment using Anti-FLAG® M2 Magnetic Beads (Millipore Sigma) according to the manufacturer's protocol with slight modifications. For each peptide, 10 μL of a 50% (v/v) suspension of magnetic beads was used. The supernatant was pipetted from the beads on a magnetic manifold. The beads were then washed twice by incubating with 100 μL of TBS (150 mM NaCl, 50 mM Tris-HCl, pH 7.6) for 10 min at room temperature then removing the supernatant each time with a magnetic manifold. The in vitro translation reactions were added to the beads and incubated at RT for 30 min with periodic agitation. The beads were washed again three times with 100 μL of TBS as described above. Peptides were eluted by incubation with 12.5 μL of 0.1 M glycine-HCl pH 2.8 for 10 minutes. The supernatant was transferred to vials and kept on ice for analysis.

The purified peptides were analyzed based on a previous protocol¹⁶. The supernatant was analyzed on an ZORBAX Eclipse XDB-C18 column (1.8 μm, 2.1×50 mm, room temperature, Agilent) using an 1290 Infinity II UHPLC (G7120AR, Agilent). The following method was used for separation: an initial hold at 95% Solvent A (0.1% formic acid in water) and 5% Solvent B (acetonitrile) for 0.5 min followed by a linear gradient from 5 to 50% Solvent B over 4.5 min at flow rate of 0.7 mL/min. Peptides were identified using LC-HRMS with an Agilent 6530 Q-TOF AJS-ESI (G6230BAR). The following parameters were used: a fragmentor voltage of 175 V, gas temperature of 300° C., gas flow rate of 12 L/min, sheath gas temperature of 350° C., sheath gas flow rate of 11 L/min, nebulizer pressure of 35 psi, skimmer voltage of 75 V, Vcap of 3500 V, and collection rate of 3 spectra/s. Expected exact masses of the major charge state for each peptide were calculated using ChemDraw 19.0 and extracted from the total ion chromatograms ±100 ppm.

Plasmids used for in vivo studies. The plasmids used to express wild-type (WT) sfGFP (pET22b-T5/lac-sfGFP) and 151TAG-sfGFP (pET22b-T5/lac-sfGFP-151TAG) in E. coli have been described⁸⁷. pET22b-T5/lac-sfGFP-200TAG was constructed from pET22b-T5/lac-sfGFP using a Q5® Site-Directed Mutagenesis Kit (NEB) with primers CS43 and CS44 (Extended Data Table 1). The synthetase/tRNA plasmid for WT MaPylRS (pMega-MaPylRS) was constructed by inserting a synthetic dsDNA fragment (pMega MaPylRS) (Extended Data Table 1) into the NotI-XhoI cut sites of a pUltra vector⁶¹ using the Gibson method⁸⁸. pMega-MaFRSA was constructed by inserting a synthetic dsDNA fragment (made by annealing primers RF48 and RF49) following inverse PCR of pMega-MaPylRS with primers RF61 and RF62 (Extended Data Table 1) using the Gibson method⁸⁸. The sequences of the plasmids spanning the inserted regions were confirmed via Sanger sequencing at the UC Berkeley DNA Sequencing Facility using primers T7 F and T7 R (Extended Data Table 1) and the complete sequence of each plasmid was confirmed by full-plasmid sequencing with Primordium Labs.

Plate reader analysis of sfGFP expression. E. coli DH10B chemically competent cells were transformed with pET22b-T5/lac-sfGFP-200TAG and either pMega-MaPylRS or pMega-MaFRSA. Colonies were picked and grown overnight in LB with the appropriate antibiotics. The following day, the OD₆₀₀ of the overnight culture was measured, and all cultures were diluted with LB to an OD₆₀₀ of 0.10 to generate a seed culture. A monomer cocktail was prepared in LB supplemented with 2 mM IPTG, 2 mM monomer 1, 2, 20, or 21, and the appropriate antibiotics at 2× final concentration (200 μg/mL carbenicillin and 100 μg/mL spectinomycin). In a 96-well plate (Corning 3904), 100 μL of the seed culture was combined with 100 μL of each monomer cocktail to bring the starting OD₆₀₀ to 0.05 and halve the concentration of the monomer cocktail. A Breathe Easy sealing membrane (Sigma-Aldrich) was applied to the top of the 96-well plate to seal it, and the plates were loaded into a Synergy HTX plate reader (BioTek). The plate was incubated at 37° C. for 24 hours with continuous shaking. At 10 minute intervals two readings were made: the absorbance at 600 nm to measure cell density, and sfGFP fluorescence with excitation at 485 nm and emission at 528 nm.

Expression and purification of sfGFP variants. Plasmids used to express sfGFP-wt and sfGFP-200TAG were co-transformed with pMega-MaPylRS or pMega-MaFRSA into DH10B or DH10B ΔaspC ΔtyrB chemically competent cells and plated onto LB agar plates supplemented with 100 μg/mL carbenicillin and 100 μg/mL spectinomycin. Colonies were picked the following day and used to inoculate 10 mL of LB supplemented with 100 μg/mL carbenicillin and 100 μg/mL spectinomycin. The cultures were incubated overnight at 37° C. with shaking at 200 rpm. The following day the 1 mL of each culture was used to inoculate 100 mL of TB or defined media (adapted from a published protocol⁵¹ with glutamate excluded and 19 other amino acids at 200 μg/mL) supplemented with 100 μg/mL carbenicillin and 100 μg/mL spectinomycin in 250 mL baffled Erlenmeyer flasks. Cultures were incubated at 37° C. with shaking at 200 rpm for ˜4 h until they reached an OD₆₀₀ of 1.0-1.2. At this point, IPTG was added to a final concentration of 1 mM and incubation was continued overnight at 37° C. with shaking at 200 rpm. Cells were harvested by centrifugation at 4303×g for 20 min at 4° C.

sfGFP variants were purified according to a published protocol⁶⁴. The following buffers were used for protein purification: Lysis/wash buffer: 50 mM sodium phosphate (pH 8), 300 mM NaCl, 20 mM imidazole; Elution buffer: 50 mM sodium phosphate (pH 8), 250 mM imidazole; Storage buffer: 50 mM sodium phosphate (pH 7), 250 mM NaCl, 1 mM DTT. 1 cOmplete Mini EDTA-free protease inhibitor tablet was added to Wash and Elution buffers immediately before use. To isolate protein, cell pellets were resuspended in 10 mL Wash buffer. The resultant cell paste was lysed at 4° C. by homogenization (Avestin Emulsiflex C3) for 5 min at 15,000-20,000 psi. The lysate was centrifuged at 4303×g for 15 min at 4° C. to separate the soluble and insoluble fractions. The soluble lysate was incubated at 4° C. with 1 mL of TALON® resin (washed with water and equilibrated with Wash buffer) for 1 h. The lysate-resin mixture was centrifuged at 4303×g for 5 min to pellet. The supernatant was removed and the protein-bound Ni-NTA agarose resin was then washed with three 5 mL aliquots of Lysis/wash buffer centrifuging between washes to pellet. The protein was eluted from Ni-NTA agarose resin by rinsing the resin five times with 1 mL Elution buffer. The elution fractions were pooled and dialyzed overnight at 4° C. into Storage buffer using 12,000-14,000 molecular weight cutoff dialysis tubing. Protein concentration was measured using the Pierce assay (CITE). Protein samples were concentrated as needed with a 110 kDa MWCO Amicon® Ultra-15 Centrifugal Filter Unit (4303×g, 4° C.) to reach a concentration of ≥0.22 mg/mL. The protein was stored at 4° C. for later analysis. Yields were between 24 and 324 mg/L when expressed in TB, and between 3.6 and 3.7 mg/L when expressed in the defined media described above. Proteins were analyzed by LC-MS as described above.

Protease digestion and fragment identification by MS. Each isolated sfGFP sample (˜10 to 25 μg) was denatured with 6 M guanidine in a 0.15 M Tris buffer at pH 7.5, followed by disulfide reduction with 8 mM dithiothreitol (DTT) at 37° C. for 30 min. The reduced sfGFP was alkylated in the presence of 14 mM iodoacetamide at 25° C. for 25 min, followed by quenching using 6 mM DTT. The reduced/alkylated protein was exchanged into ˜40 μL of 0.1 M Tris buffer at pH 7.5 using a Microcon 10-kDa membrane, followed by addition of 2.5 μg endoproteinase Glu-C (in a 0.25 μg/μL solution) directly to the membrane to achieve an enzyme-to-substrate ratio of at least 1:10. After 3 hours at 37° C., the digestion was quenched with an equal volume of 0.25 M acetate buffer (pH 4.8) containing 6 M guanidine. Peptide fragments were collected by spinning down through the membrane and subjected to LC-MS/MS analysis.

LC-MS/MS analysis was performed on an Agilent 1290-II HPLC directly connected to a Thermo Fisher Q Exactive HF high-resolution mass spectrometer. Peptides were separated on a Waters HSS T3 reversed-phase column (2.1×150 mm) at 50° C. with a 70 min acetonitrile gradient (0.5% to 35%) containing 0.1% formic acid in the mobile phase, and a total flow rate of 0.25 mL/min. The MS data were collected at 120 k resolution setting, followed by data-dependent higher-energy collision dissociation (HCD) MS/MS at a normalized collision energy of 25%.

Proteolytic peptides were identified and quantified on MassAnalyzer, an in-house developed program⁸⁹ (available in Biopharma Finder™ from Thermo Fisher). The program performs feature extraction, peptide identification, retention time alignment⁹⁰, and peak integration in an automated fashion.

### REFERENCES

1. Lutz, J., Ouchi, M., Liu, D. & Sawamoto, M. Sequence-Controlled Polymers. Science 341, 628-+ (2013).
2. Barnes, J. et al. Iterative exponential growth of stereo- and sequence-controlled polymers. Nat. Chem. 7, 810-815 (2015).
3. Fahnestock, S. & Rich, A. Ribosome-catalyzed polyester formation. Science 173, 340-343 (1971).
4. Ohta, A., Murakami, H., Higashimura, E. & Suga, H. Synthesis of polyester by means of genetic code reprogramming. Chem. Biol. 14, 1315-1322 (2007).
5. Katoh, T., Iwane, Y. & Suga, H. Logical engineering of D-arm and T-stem of tRNA that enhances d-amino acid incorporation. Nucleic Acids Res. 45, 12601-12610 (2017).
6. Fujino, T., Goto, Y., Suga, H. & Murakami, H. Ribosomal Synthesis of Peptides with Multiple β-Amino Acids. J. Am. Chem. Soc. 138, 1962-1969 (2016).
7. Katoh, T. & Suga, H. Ribosomal Incorporation of Consecutive β-Amino Acids. J. Am. Chem. Soc. 140, 12159-12167 (2018).
8. Adaligil, E., Song, A., Hallenbeck, K. K., Cunningham, C. N. & Fairbrother, W. J. Ribosomal Synthesis of Macrocyclic Peptides with β²- and β^{2,3}-Homo-Amino Acids for the Development of Natural Product-Like Combinatorial Libraries. ACS Chem. Biol. 16, 1011-1018 (2021).
9. Katoh, T., Sengoku, T., Hirata, K., Ogata, K. & Suga, H. Ribosomal synthesis and de novo discovery of bioactive foldamer peptides containing cyclic β-amino acids. Nat. Chem. 12, 1081-1088 (2020).
10. Katoh, T. & Suga, H. Ribosomal Elongation of Cyclic γ-Amino Acids using a Reprogrammed Genetic Code. J. Am. Chem. Soc. 142, 4965-4969 (2020).
11. Adaligil, E., Song, A., Cunningham, C. N. & Fairbrother, W. J. Ribosomal Synthesis of Macrocyclic Peptides with Linear γ⁴- and β-Hydroxy-γ⁴-amino Acids. ACS Chem. Biol. 16, 1325-1331 (2021).
12. Lee, J., Schwarz, K. J., Kim, D. S., Moore, J. S. & Jewett, M. C. Ribosome-mediated polymerization of long chain carbon and cyclic amino acids into peptides in vitro. Nat. Commun. 11, 4304 (2020).
13. Katoh, T. & Suga, H. Consecutive Ribosomal Incorporation of α-Aminoxy/α-Hydrazino Acids with 1/d-Configurations into Nascent Peptide Chains. J. Am. Chem. Soc. 143, 18844-18848 (2021).
14. Takatsuji, R. et al. Ribosomal Synthesis of Backbone-Cyclic Peptides Compatible with In Vitro Display. J. Am. Chem. Soc. 141, 2279-2287 (2019).
15. Katoh, T. & Suga, H. Ribosomal Elongation of Aminobenzoic Acid Derivatives. J. Am. Chem. Soc. 142, 16518-16522 (2020).
16. Ad, O. et al. Translation of Diverse Aramid- and 1,3-Dicarbonyl-peptides by Wild Type Ribosomes in Vitro. ACS Cent. Sci. 5, 1289-1294 (2019).
17. Sievers, A., Beringer, M., Rodnina, M. V. & Wolfenden, R. The ribosome as an entropy trap. Proc. Natl. Acad. Sci. U.S.A. 101, 7897 (2004).
18. England, P. M., Zhang, Y., Dougherty, D. A. & Lester, H. A. Backbone Mutations in Transmembrane Domains of a Ligand-Gated Ion Channel: Implications for the Mechanism of Gating. Cell 96, 89-98 (1999).
19. Guo, J., Wang, J., Anderson, J. C. & Schultz, P. G. Addition of an α-Hydroxy Acid to the Genetic Code of Bacteria. Angew. Chem. 120, 734-737 (2008).
20. Kobayashi, T., Yanagisawa, T., Sakamoto, K. & Yokoyama, S. Recognition of Non-α-amino Substrates by Pyrrolysyl-tRNA Synthetase. J. Mol. Biol. 385, 1352-1360 (2009).
21. Li, Y.-M. et al. Ligation of Expressed Protein α-Hydrazides via Genetic Incorporation of an α-Hydroxy Acid. ACS Chem. Biol. 7, 1015-1022 (2012).
22. Melo Czekster, C., Robertson, W. E., Walker, A. S., Söll, D. & Schepartz, A. In Vivo Biosynthesis of a β-Amino Acid-Containing Protein. J. Am. Chem. Soc. 138, 5194-5197 (2016).
23. Chen, S., Ji, X., Gao, M., Dedkova, L. M. & Hecht, S. M. In Cellulo Synthesis of Proteins Containing a Fluorescent Oxazole Amino Acid. J. Am. Chem. Soc. 141, 5597-5601 (2019).
24. Liu, C. C. & Schultz, P. G. Adding New Chemistries to the Genetic Code. Annu. Rev. Biochem. 79, 413-444 (2010).
25. Wan, W., Tharp, J. M. & Liu, W. R. Pyrrolysyl-tRNA synthetase: An ordinary enzyme but an outstanding genetic code expansion tool. Biochim. Biophys. Acta BBA—Proteins Proteomics 1844, 1059-1070 (2014).
26. Vargas-Rodriguez, O., Sevostyanova, A., Soil, D. & Crnković, A. Upgrading aminoacyl-tRNA synthetases for genetic code expansion. Curr. Opin. Chem. Biol. 46, 115-122 (2018).
27. Italia, J. S. et al. Mutually Orthogonal Nonsense-Suppression Systems and Conjugation Chemistries for Precise Protein Labeling at up to Three Distinct Sites. J. Am. Chem. Soc. 141, 6204-6212 (2019).
28. Chin, J. W. Expanding and reprogramming the genetic code. Nature 550, 53-60 (2017).
29. Srinivasan Gayathri, James Carey M., & Krzycki Joseph A. Pyrrolysine Encoded by UAG in Archaea: Charging of a UAG-Decoding Specialized tRNA. Science 296, 1459-1462 (2002).
30. Ambrogelly, A., Palioura, S. & Soll, D. Natural expansion of the genetic code. Nat. Chem. Biol. 3, 29-35 (2007).
31. Wang, L., Brock, A., Herberich, B. & Schultz, P. G. Expanding the Genetic Code of Escherichia coli. Science 292, 498-500 (2001).
32. Kobayashi, T. et al. Structural basis for orthogonal tRNA specificities of tyrosyl-tRNA synthetases for genetic code expansion. Nat. Struct. Biol. 10, 8 (2003).
33. Yanagisawa, T. et al. Crystallographic Studies on Multiple Conformational States of Active-site Loops in Pyrrolysyl-tRNA Synthetase. J. Mol. Biol. 378, 634-652 (2008).
34. Borrel, G. et al. Genome Sequence of ‘Candidatus Methanomethylophilus alvus’ Mx1201, a Methanogenic Archaeon from the Human Gut Belonging to a Seventh Order of Methanogens. J. Bacteriol. 194, 6944-6945 (2012).
35. Willis, J. C. W. & Chin, J. W. Mutually orthogonal pyrrolysyl-tRNA synthetase/tRNA pairs. Nat. Chem. 10, 831-837 (2018).
36. Wang, Y.-S. et al. The de novo engineering of pyrrolysyl-tRNA synthetase for genetic incorporation of l-phenylalanine and its derivatives. Mol. Biosyst. 7, 714 (2011).
37. Wang, Y.-S., Fang, X., Wallace, A. L., Wu, B. & Liu, W. R. A Rationally Designed Pyrrolysyl-tRNA Synthetase Mutant with a Broad Substrate Spectrum. J. Am. Chem. Soc. 134, 2950-2953 (2012).
38. Herring, S. et al. The amino-terminal domain of pyrrolysyl-tRNA synthetase is dispensable in vitro but required for in vivo activity. FEBS Lett. 581, 3197-3203 (2007).
39. McMurry, J. L. & Chang, M. C. Y. Fluorothreonyl-tRNA deacylase prevents mistranslation in the organofluorine producer Streptomyces cattleya. Proc. Natl. Acad. Sci. 114, 11920-11925 (2017).
40. Findly, D., Herries, D. G., Mathias, A. P., Rabin, B. R. & Ross, C. A. The Active Site and Mechanism of Action of Bovine Pancreatic Ribonuclease. Nature 190, 781-784 (1961).
41. Griffin, B. E., Jarman, M., Reese, C. B., Sulston, J. E. & Trentham, D. R. Some Observations Relating to Acyl Mobility in Aminoacyl Soluble Ribonucleic Acids*. Biochemistry 5, 3638-3649 (1966).
42. Gottfried-Lee, I., Perona, J. J., Karplus, P. A., Mehl, R. A. & Cooley, R. B. Structures of Methanomethylophilus alvus Pyrrolysine tRNA-Synthetases Support the Need for De Novo Selections When Altering the Substrate Specificity. ACS Chem. Biol. (2022) doi:10.1021/acschembio.2c00640.
43. Stepanov, V. G., Moor, N. A., Ankilova, V. N. & Lavrik, O. I. Phenylalanyl-tRNA synthetase from Thermus thermophilus can attach two molecules of phenylalanine to tRNA^{Phe}. FEBS Lett. 311, 192-194 (1992).
44. Wang, B., Zhou, J., Lodder, M., Anderson, R. D. & Hecht, S. M. Tandemly Activated tRNAs as Participants in Protein Synthesis. J. Biol. Chem. 281, 13865-13868 (2006).
45. Englert, M. et al. Aminoacylation of tRNA 2′- or 3′-hydroxyl by phosphoseryl- and pyrrolysyl-tRNA synthetases. FEBS Lett. 587, 3360-3364 (2013).
46. Ko, J. et al. Pyrrolysyl-tRNA synthetase variants reveal ancestral aminoacylation function. FEBS Lett. 587, 3243-3248 (2013).
47. Xuan, W. et al. Site-Specific Incorporation of a Thioester Containing Amino Acid into Proteins. ACS Chem. Biol. 13, 578-581 (2018).
48. Hwang, S., Lee, N., Cho, S., Palsson, B. & Cho, B.-K. Repurposing Modular Polyketide Synthases and Non-ribosomal Peptide Synthetases for Novel Chemical Biosynthesis. Front. Mol. Biosci. 7, (2020).
49. Muir, T., Sondhi, D. & Cole, P. Expressed protein ligation: A general method for protein engineering. Proc. Natl. Acad. Sci. U.S.A. 95, 6705-6710 (1998).
50. Varshney, U., Lee, C. P., Seong, B. L. & RajBhandary, U. L. Mutants of initiator tRNA that function both as initiators and elongators. J. Biol. Chem. 266, 18018-18024 (1991).
51. Tharp, J. M. et al. Initiation of Protein Synthesis with Non-Canonical Amino Acids In Vivo. Angew. Chem. Int. Ed. 59, 3122-3126 (2020).
52. Cestari, I. & Stuart, K. A Spectrophotometric Assay for Quantitative Measurement of Aminoacyl-tRNA Synthetase Activity. J. Biomol. Screen. 18, 490-497 (2013).
53. Wang, Y.-S. et al. Genetic Incorporation of Twelve meta-Substituted Phenylalanine Derivatives Using a Single Pyrrolysyl-tRNA Synthetase Mutant. ACS Chem. Biol. 8, 405-415 (2013).
54. Guo, L.-T. et al. Polyspecific pyrrolysyl-tRNA synthetases from directed evolution. Proc. Natl. Acad. Sci. 111, 16724-16729 (2014).
55. Tharp, J. M., Wang, Y.-S., Lee, Y.-J., Yang, Y. & Liu, W. R. Genetic Incorporation of Seven ortho-Substituted Phenylalanine Derivatives. ACS Chem. Biol. 9, 884-890 (2014).
56. Seki, E., Yanagisawa, T., Kuratani, M., Sakamoto, K. & Yokoyama, S. Fully Productive Cell-Free Genetic Code Expansion by Structure-Based Engineering of Methanomethylophilus alvus Pyrrolysyl-tRNA Synthetase. ACS Synth. Biol. 9, 718-732 (2020).
57. Kavran, J. M. et al. Structure of pyrrolysyl-tRNA synthetase, an archaeal enzyme for genetic code innovation. Proc. Natl. Acad. Sci. 104, 11268-11273 (2007).
58. Englert, M. et al. Probing the active site tryptophan of Staphylococcus aureus thioredoxin with an analog. Nucleic Acids Res. 43, 11061-11067 (2015).
59. Laursen, B. S., Sørensen, H. P., Mortensen, K. K. & Sperling-Petersen, H. U. Initiation of Protein Synthesis in Bacteria. Microbiol. Mol. Biol. Rev. 69, 101-123 (2005).
60. Lee, J. et al. Expanding the limits of the second genetic code with ribozymes. Nat. Commun. 10, 5097 (2019).
61. Chatterjee, A., Sun, S. B., Furman, J. L., Xiao, H. & Schultz, P. G. A Versatile Platform for Single- and Multiple-Unnatural Amino Acid Mutagenesis in Escherichia coli. Biochemistry 52, 1828-1837 (2013).
62. Goldman, E. & Jakubowski, H. Uncharged tRNA, protein synthesis, and the bacterial stringent response. Mol. Microbiol. 4, 2035-2040 (1990).
63. Young, T. S., Ahmad, I., Yin, J. A. & Schultz, P. G. An Enhanced System for Unnatural Amino Acid Mutagenesis in E. coli. J. Mol. Biol. 395, 361-374 (2010).
64. González, S. S. et al. Genetic Code Expansion in the Engineered Organism Vmax X2: High Yield and Exceptional Fidelity. ACS Cent. Sci. 7, 1500-1507 (2021).
65. Bindman, N. A., Bobeica, S. C., Liu, W. R. & van der Donk, W. A. Facile Removal of Leader Peptides from Lanthipeptides by Incorporation of a Hydroxy Acid. J. Am. Chem. Soc. 137, 6975-6978 (2015).
66. Nivina, A., Yuet, K., Hsu, J. & Khosla, C. Evolution and Diversity of Assembly-Line Polyketide Synthases. Chem. Rev. 119, 12524-12547 (2019).
67. Tsai, S. The Structural Enzymology of Iterative Aromatic Polyketide Synthases: A Critical Comparison with Fatty Acid Synthases. in Annual Review of Biochemistry (ed. Kornberg, R.) vol. 87 503-531 (2018).
68. Walsh, C. T., O'Brien, R. V. & Khosla, C. Nonproteinogenic amino acid building blocks for nonribosomal peptide and hybrid polyketide scaffolds. Angew. Chem. Int. Ed. 52, 7098-7124 (2013).
69. Ostrov, N. et al. Design, synthesis, and testing toward a 57-codon genome. Science 353, 819-822 (2016).
70. Fredens, J. et al. Total synthesis of Escherichia coli with a recoded genome. Nature 569, 514-518 (2019).
71. Gibson, D. G. et al. Enzymatic assembly of DNA molecules up to several hundred kilobases. Nat. Methods 6, 343-345 (2009).
72. Chung, C. T., Niemela, S. L. & Miller, R. H. One-step preparation of competent Escherichia coli: transformation and storage of bacterial cells in the same solution. Proc. Natl. Acad. Sci. 86, 2172-2175 (1989).
73. Bradford, M. M. A Rapid and Sensitive Method for the Quantitation of Microgram Quantities of Protein Utilizing the Principle of Protein-Dye Binding. 7.
74. Baklanov, M. Effect on DNA transcription of nucleotide sequences upstream to T7 promoter. Nucleic Acids Res. 24, 3659-3660 (1996).
75. Kao, C., Zheng, M. & Rüdisser, S. A simple and efficient method to reduce nontemplated nucleotide addition at the 3′ terminus of RNAs transcribed by T7 RNA polymerase. RNA 5, 1268-1272 (1999).
76. Noncanonical Amino Acids: Methods and Protocols. vol. 1728 (Springer New York, 2018).
77. RNA Molecular Weight Calculator|AAT Bioquest. https://www.aatbio.com/tools/calculate-RNA-molecular-weight-mw/.
78. Sprinzl, M. & Cramer, F. The -C-C-A End of tRNA and Its Role in Protein Biosynthesis. in Progress in Nucleic Acid Research and Molecular Biology vol. 22 1-69 (Elsevier, 1979).
79. Kabsch, W. XDS. Acta Crystallogr. D Biol. Crystallogr. 66, 125-132 (2010).
80. Evans, P. R. An introduction to data reduction: space-group determination, scaling and intensity statistics. Acta Crystallogr. D Biol. Crystallogr. 67, 282-292 (2011).
81. Evans, P. R. & Murshudov, G. N. How good are my data and what is the resolution? Acta Crystallogr. D Biol. Crystallogr. 69, 1204-1214 (2013).
82. Bunkóczi, G. et al. Phaser.MRage: automated molecular replacement. Acta Crystallogr. D Biol. Crystallogr. 69, 2276-2286 (2013).
83. Emsley, P., Lohkamp, B., Scott, W. G. & Cowtan, K. Features and development of Coot. Acta Crystallogr. D Biol. Crystallogr. 66, 486-501 (2010).
84. Adams, P. D. et al. PHENIX: a comprehensive Python-based system for macromolecular structure solution. Acta Crystallogr. D Biol. Crystallogr. 66, 213-221 (2010).
85. Liebschner, D. et al. Macromolecular structure determination using X-rays, neutrons and electrons: recent developments in Phenix. Acta Crystallogr. Sect. Struct. Biol. 75, 861-877 (2019).
86. Schrödinger, LLC. The PyMOL Molecular Graphics System, Version 1.8. (2015).
87. Grasso, K. T. et al. A Facile Platform to Engineer Escherichia coli Tyrosyl-tRNA Synthetase Adds New Chemistries to the Eukaryotic Genetic Code, Including a Phosphotyrosine Mimic. ACS Cent. Sci. 8, 483-492 (2022).
88. Gibson, D. G. et al. Enzymatic assembly of DNA molecules up to several hundred kilobases. Nat. Methods 6, 343-345 (2009).
89. Zhang, Z. Large-Scale Identification and Quantification of Covalent Modifications in Therapeutic Proteins. Anal. Chem. 81, 8354-8364 (2009).
90. Zhang, Z. Retention Time Alignment of LC/MS Data by a Divide-and-Conquer Algorithm. J. Am. Soc. Mass Spectrom. 23, 764-772 (2012).
91. Herold, S., Bafaluy, D. & Muñiz, K. Anodic benzylic C(sp3)-H amination: unified access to pyrrolidines and piperidines. Green Chem. 20, 3191-3196 (2018).
92. Matveeva, E. D. et al. Syntheses of Compounds Active toward Glutamate Receptors: II. Synthesis of Spiro Hydantoins of the Indan Series. Russ. J. Org. Chem. 38, 1769-1774 (2002).
93. Madeira, F. et al. The EMBL-EBI search and sequence analysis tools APIs in 2019. Nucleic Acids Res. 47, W636-W641 (2019).

### Supplementary Data Information

### Materials

Materials were sourced from the following suppliers: Agilent Technologies (Santa Clara, CA): BL21-Gold (DE3) Competent Cells; Alfa Aesar (Ward Hill, MA): N(ε)-Boc-L-lysine (L-BocK), N(ε)-Boc-D-lysine (D-BocK), 3-Methylbenzyl bromide, 3-(Trifluoromethyl)benzyl bromide, 3-Bromobenzyl bromide, L-lysine monohydrochloride; AmericanBio (Canton, MA): carbenicillin, glycerol, isopropyl β-D-1-thiogalactopyranoside (IPTG), HEPES, magnesium chloride (1 M solution), sodium acetate buffer (3 M, pH 5.2), EDTA-Na (0.5 M solution, pH 8.0); BACHEM (Torrance, CA): N-methyl-L-phenylalanine, N-formyl-L-phenylalanine; BioRad (Hercules, CA): Any kD™ Mini-PROTEAN® TGX™ Precast Protein Gels (product 4569033), 10% Mini-PROTEAN® TBE-Urea Gel (product 4566036), Micro Bio-Spin™ P-30 Gel Columns, Tris Buffer RNase-free (product 7326250), Precision Plus Protein™ Dual Color Standards (product 1610374); BioWorld (Dublin, OH): Luria-Bertani broth (LB), Terrific broth (TB); Cayman Chemical (Ann Arbor, MI): α-mercapto-benzenepropanoic acid; Cytiva Life Sciences (Marlborough, MA): Superdex® 75 Increase 10/300 GL column, HiLoad® 16/600 Superdex® 200 pg column; Decon Labs (King of Prussia, PA): 200 proof ethanol; Echelon Biosciences (Salt Lake City, UT): malachite green solution; Fisher Scientific (Pittsburgh, PA): Agar, sodium hydroxide, potassium hydroxide, sodium chloride, potassium chloride, calcium chloride, dithiothreitol (DTT), 50% polyethylene glycol 3350 solution, acetonitrile Optima™ LC/MS Grade, Tris base, ethylenediaminetetraacetic acid (free acid); Frontier Scientific (Logan, UT): L-phenylalanine, L-aspartic acid, L-valine, L-tyrosine; Honeywell (Charlotte, NC): 1,1,1,3,3,3-Hexafluoro-2-propanol, LC-MS Grade (HFIP); Integrated DNA Technologies (Coralville, IA): RF31, RF32, RF33, Ma-PylT-F, Ma-PylT-R; Invitrogen (Waltham, MA): SYBR™ Safe DNA Gel Stain; J. T. Baker—Avantor (Radnor, PA): sodium phosphate, chloroform, boric acid, hydrochloric acid, dimethylsulfoxide (DMSO); MilliporeSigma (Burlington, MA): β-mercaptoethanol (BME), imidazole, cesium chloride, adenosine 5′-(β,γ-imido)triphosphate lithium salt hydrate (AMP-PNP), ribonuclease A from bovine pancrease (RNAse A), acidic phenol (Phenol Saturated Citrate Buffered pH 4.5), ethanol, spermidine, guanosine monophosphate (GMP), bovine serum albumin (BSA), polyethylene glycol 8000, 6-(Boc-amino)hexanoic acid (BocAhx), (S)-(−)-3-phenyllactic acid (3-PLA), 2-benzylmalonic acid (2-BMA), 4-(Boc-amino)butyl bromide, diethyl malonate, tetrahydrofuran anhydrous, sodium hydride 60% dispersion in mineral oil, sodium sulfate anhydrous, diethylether, Thrombin CleanCleave™ Kit, 10 kDa MWCO Amicon® Ultra-15 Centrifugal Filter Unit, Anti-FLAG® M2 Magnetic Beads, basic phenol (Phenol solution equilibrated with 10 mM Tris HCl, pH 8.0, 1 mM EDTA); MP Biomedicals (Irvine, CA): D-phenylalanine, glycine hydrochloride; New England BioLabs (Ipswich, MA): NdeI restriction enzyme, OneTaq® Quick-Load® 2× Master Mix, nucleotide triphosphate solutions, Low Range ssRNA Ladder, PURExpress® Δ (aa, tRNA) Kit, Q5® High-Fidelity 2× Master Mix; PepTech (Bedford, MA): 3-Trifluoromethyl-L-phenylalanine; Promega (Madison, WI): RQ1 RNase-Free DNase Qiagen (Germantown, MD): Ni-NTA Agarose resin; Ricca Chemical Company (Arlington, TX): formic acid LCMS grade, triethylamine (TEA) LCMS grade; Roche (Basel, Switzerland): cOmplete™ Mini EDTA-free Protease Inhibitor Cocktail; Takara Bio (San Jose, CA): TALON® Metal Affinity Resin; Teledyne ISCO (Lincoln, NE): 65 g RediSep® Disposable Sample Load Cartridge; Tokyo Chemical Industry (Portland, OR): 3-phenylpropanoic acid (3-PLA).

### Synthesis Notes

Synthesis of meta-substituted 2-benzylmalonates 17-19 and malonate 16.

General. Alkylation reactions to synthesize 20-23 as well as hydrolysis reactions to synthesize 16-19 were based on published methods^{91,92}. All reagents and solvents were used as received from commercial suppliers, unless indicated otherwise. Alkylation reactions were carried out with exclusion of air and moisture. Room temperature is considered 20-23° C. Stirring was achieved with Teflon-coated magnetic stir bars. TLC was performed on glass-backed silica gel plates (median pore size 60 Å) and visualized using UV light at 254 nm or staining with iodine. Column chromatography was performed on an Isco Teledyne Combiflash Nextgen 300+ instrument using pre-packed Redi-sep Gold silica gel cartridges (particle diameter 20-40 μM, pore diameter 60 Å). The eluents are given in brackets. Mass spectrometry was performed on an LTQ FT-ICR mass spectrometer equipped with an electrospray ionization source (Finnigan LTQ FT, Thermo Fisher Scientific, Waltham, MA) operated in either positive or negative ion mode. ¹H NMR spectra NMR data were acquired at 298 K using a 500 MHz Bruker Avance Neo NMR spectrometer that was equipped with a 5 mm iProbe or a 400 MHz Bruker Avance I spectrometer equipped with a 5 mm BBO Smart Probe. The experiments were conducted using the default Bruker NMR parameters and data was time-averaged until a sufficient level of sensitivity was achieved. ¹H NMR data was calibrated by using the residual peak of the solvent as the internal standard (CDCl₃: δ_{H}=7.26 ppm; CD₃OD: δ_{H}=3.31 ppm). All coupling constants are recorded in Hz. NMR spectra were processed with MestReNova v14.1.2-25024 software using the baseline and phasing correction features. Multiplicities and coupling constants were calculated using the multiplet analysis feature with manual intervention as necessary.

Diethyl 2-(3-methylbenzyl)malonate (20) Diethyl malonate (500.57 mg, 3.125 mmol, 1.05 equiv.) was added dropwise to a suspension of 60% NaH on mineral oil (125 mg, 3.125 mmol, 1.05 equiv.) in 6 mL dry THF at 0° C. After 20 min, 3-methylbenzyl bromide (550.86 mg, 2.97 mmol, 1 equiv.) was added in one portion and the reaction mixture was refluxed overnight. The next day, the reaction was cooled and quenched by the addition of H₂O. Et₂O was added and the aqueous layer was extracted three times with Et₂O. The combined organic layers were dried over Na₂SO₄ then evaporated to dryness under reduced pressure. The crude product was purified by flash chromatography on SiO₂ [eluent: EtOAc/hexane (5% then 10% then 15% then 20%)] to obtain pure diethyl 2-(3-methylbenzyl)malonate 20 as a clear liquid. Yield 20.6%. ¹H NMR (500 MHz, CDCl₃) δ 7.19 (t, J=7.8 Hz, 1H), 7.07-7.00 (m, 3H), 4.19 (qd, J=7.1, 2.8 Hz, 4H), 3.65 (t, J=7.8 Hz, 1H), 3.20 (d, J=7.8 Hz, 2H), 2.34 (s, 3H), 1.24 (t, J=7.1 Hz, 6H). HR-EI-MS [M+H]+: calculated for C15H21O4+, m/z 265.1434, found m/z 265.1395.

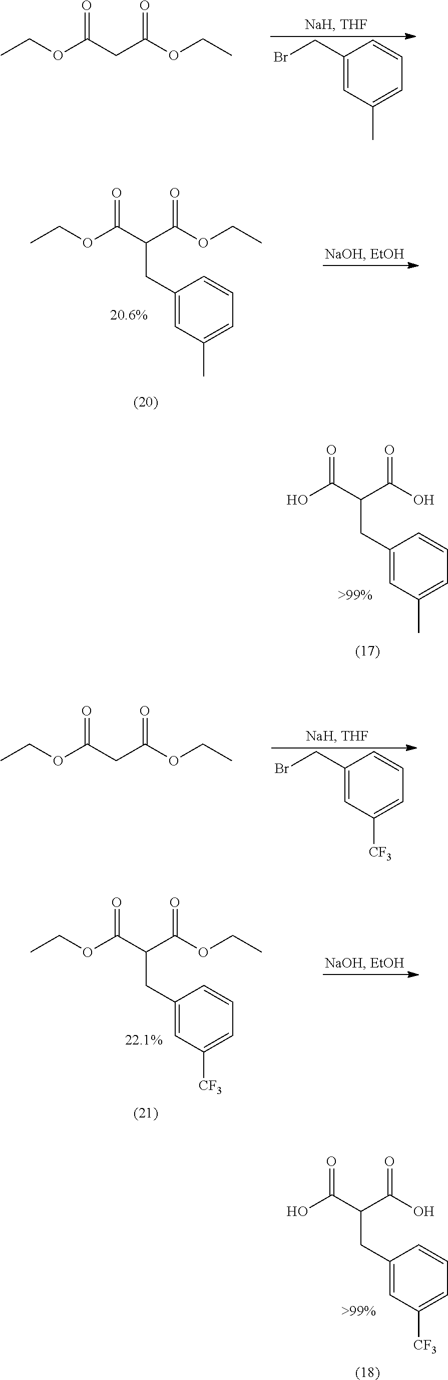

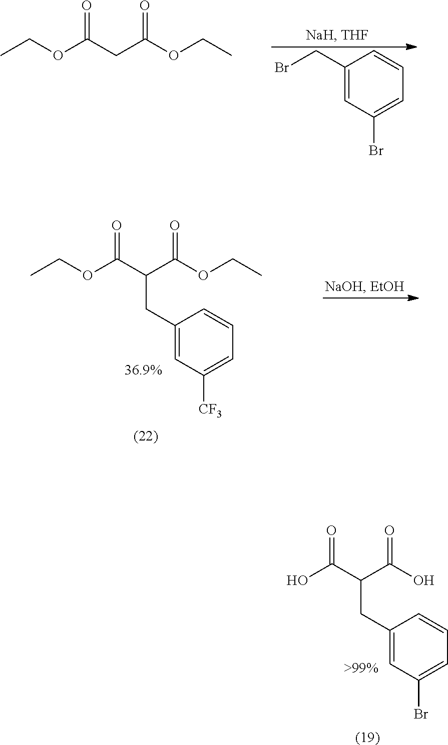 [SMILES: CCOC(=O)CC(=O)OCC ; BrCc1cccc(Br)c1 ; CCOC(=O)C(Cc1cccc(C(F)(F)F)c1)C(=O)OCC ; "(22)" ; O=C(O)C(Cc1cccc(Br)c1)C(=O)O ; "(19)"]

2-(3-methylbenzyl)malonic acid (17) Diethyl 2-(3-methylbenzyl)malonate 20 (100 mg, 0.362 mmol) was dissolved in 1 mL of ethanol then added dropwise to 375 μL of 6.67 M NaOH. The mixture was stirred for 5 h at 60° C. The solution was then cooled to 0° C., carefully acidified to pH 1 with 1 N HCl, and extracted with 5 portions of Et₂O. The combined extracts were washed with a saturated aqueous solution of NaCl, dried over Na₂SO₄ and evaporated to dryness. 2-(3-methylbenzyl)malonic acid was dissolved in 1:1 H₂O:MeCN and lyophilized to give a white solid. Yield >99%. ¹H NMR (500 MHz, MeOD) δ 7.04 (t, J=7.6 Hz, 1H), 6.95 (s, 1H), 6.94-6.88 (m, 2H), 3.49 (t, J=7.8 Hz, 1H), 3.01 (d, J=7.7 Hz, 2H), 2.20 (s, 3H). HR-ESI-MS [M−H]−: calculated for C11H11O4, m/z 207.0663, found m/z 207.0661.

Diethyl 2-(3-(trifluoromethyl)benzyl)malonate (21) Diethyl malonate (500.57 mg, 3.125 mmol, 1.05 equiv.) was added dropwise to a suspension of 60% NaH on mineral oil (125 mg, 3.125 mmol, 1.05 equiv.) in 6 mL dry THF at 0° C. After 20 min, 3-(trifluoromethyl)benzyl bromide (711.377 mg, 2.97 mmol, 1 equiv.) was added in one portion and the reaction mixture was refluxed overnight. The next day, the reaction was cooled and quenched by the addition of H₂O. Et₂O was added and the aqueous layer was extracted three times with Et₂O. The combined organic layers were then dried over Na₂SO₄ then evaporated to dryness under reduced pressure. The crude product was purified by flash chromatography on SiO₂ [eluent: EtOAc/hexane (5% then 10% then 15% then 20%)] to obtain pure diethyl 2-(3-(trifluoromethyl)benzyl)malonate as a clear liquid. Yield 22.1%. ¹H NMR (400 MHz, CDCl3) δ 7.56-7.49 (m, 2H), 7.45 (p, J=2.1 Hz, 2H), 4.21 (qd, J=7.2, 1.7 Hz, 4H), 3.68 (t, J=7.8 Hz, 1H), 3.31 (d, J=7.9 Hz, 2H), 1.25 (t, J=7.1 Hz, 6H). HR-EI-MS [M+H]+: calculated for C15H18F3O4+, m/z 319.1152, found m/z 319.1112.

2-(3-(trifluoromethyl)benzyl)malonic acid (18) Diethyl 2-(3-(trifluoromethyl)benzyl)malonate (100 mg, 0.314 mmol) was dissolved in 1 mL of ethanol then added dropwise to 375 μL of 6.67 M NaOH. The mixture was stirred for 5 h at 60° C. The solution was then cooled to 0° C., carefully acidified to pH 1 with 1 N HCl, and extracted with 5 portions of Et₂O. The combined extracts were washed with a saturated solution of NaCl, dried over Na₂SO₄, and evaporated to dryness. 2-(3-(trifluoromethyl)benzyl)malonic acid was dissolved in 1:1 H₂O:MeCN and lyophilized to give a white solid. Yield >99%. ¹H NMR (500 MHz, MeOD) δ 7.60-7.46 (m, 4H), 3.69 (t, J=7.8 Hz, 1H), 3.26 (d, J=7.7 Hz, 2H). HR-ESI-MS [M−H]−: calculated for C11H8F3O4, m/z 261.0380, found m/z 261.0377.

Diethyl 2-(3-bromobenzyl)malonate (22) Diethyl malonate (500.57 mg, 3.125 mmol, 1.05 equiv.) was added dropwise to a suspension of 60% NaH on mineral oil (125 mg, 3.125 mmol, 1.05 equiv.) in 6 mL dry THF at 0° C. After 20 min, 3-bromobenzyl bromide (743.91 mg, 2.97 mmol, 1 equiv.) was added in one portion and the reaction mixture was refluxed overnight. The next day, the reaction was cooled and quenched by the addition of H₂O. Et₂O was added and the aqueous layer was extracted three times with Et₂O. The combined organic layers were then dried over Na₂SO₄ then evaporated to dryness under reduced pressure. The crude product was purified by flash chromatography on SiO₂ [eluent: EtOAc/hexane (5% then 10% then 15% then 20%)] to obtain pure diethyl 2-(3-(trifluoromethyl)benzyl)malonate as a clear liquid. Yield 22.1%. ¹H NMR (400 MHz, CDCl3) δ 7.39-7.32 (m, 2H), 7.19-7.10 (m, 2H), 4.17 (qd, J=7.1, 1.3 Hz, 4H), 3.61 (t, J=7.8 Hz, 1H), 3.18 (d, J=7.9 Hz, 2H), 1.22 (t, J=7.1 Hz, 6H). HR-EI-MS [M+H]+: calculated for C14H18BrO4, m/z 329.0383, found m/z 329.0343.

2-(3-bromobenzyl)malonic acid (19) Diethyl 2-(3-bromobenzyl)malonate 22 (100 mg, 0.305 mmol) was dissolved in 1 mL of ethanol then added dropwise to 375 μL of 6.67 M NaOH. The mixture was stirred for 5 h at 60° C. The solution was then cooled to 0° C., carefully acidified to pH 1 with 1 N HCl, and extracted with 5 portions of Et₂O. The combined extracts were washed with a saturated solution of NaCl, dried over Na₂SO₄, and evaporated to dryness. 2-(3-bromobenzyl)malonic acid was dissolved in 1:1 H₂O:MeCN and lyophilized to give a white solid. Yield >99%. ¹H NMR (500 MHz, MeOD) δ 7.33 (t, J=1.9 Hz, 1H), 7.26 (dt, J=7.7, 1.7 Hz, 1H), 7.16-7.06 (m, 2H), 3.52 (t, J=7.8 Hz, 1H), 3.04 (d, J=7.8 Hz, 2H). HR-ESI-MS [M−H]−: calculated for C10H8BrO4, m/z 270.9611, found m/z 270.9609.

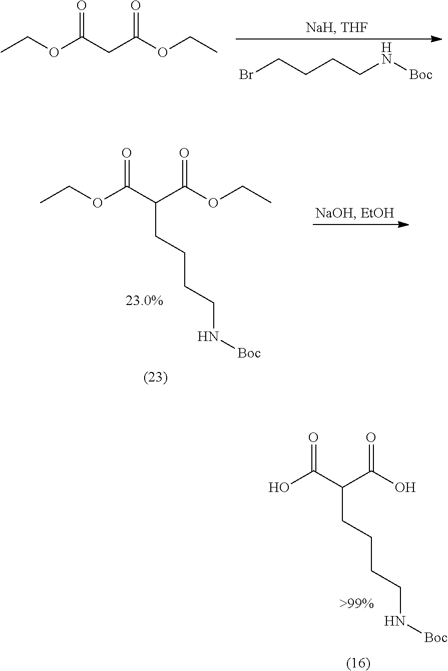

Diethyl 2-(4-((tert-butoxycarbonyl)amino)butyl)malonate (23) Diethyl malonate (166.86 mg, 1.04 mmol, 1.05 equiv.) was added dropwise to a suspension of 60% NaH on mineral oil (41.67 mg, 1.04 mmol, 1.05 equiv.) in 6 mL dry THF at 0° C. After 20 min, 4-(Boc-amino)butyl bromide (250.17 mg, 0.99 mmol, 1 equiv.) was added in one portion and the reaction mixture was refluxed overnight. The next day, the reaction was cooled and quenched by the addition of H₂O. Et₂O was added and the aqueous layer was extracted three times with Et₂O. The combined organic layers were then dried over Na₂SO₄ then evaporated to dryness under reduced pressure. The crude product was purified by flash chromatography on SiO₂ [eluent: EtOAc/hexane (5% then 10% then 15% then 20%)] to obtain pure diethyl 2-(4-((tert-butoxycarbonyl)amino)butyl)malonate as a clear liquid. Yield 22.1%. ¹H NMR (500 MHz, CDCl3) δ 4.44 (s, 1H), 4.13 (qd, J=7.1, 2.0 Hz, 4H), 3.24 (t, J=7.5 Hz, 1H), 3.04 (q, J=6.7 Hz, 2H), 1.87-1.79 (m, 2H), 1.44 (p, J=7.3 Hz, 2H), 1.37 (s, 9H), 1.28 (tt, J=10.5, 6.3 Hz, 2H), 1.20 (t, J=7.1 Hz, 6H).

2-(4-((tert-butoxycarbonyl)amino)butyl)malonic acid (16) Diethyl 2-(4-((tert-butoxycarbonyl)amino)butyl)malonate (72 mg, 0.22 mmol) was dissolved in 1 mL of ethanol then added dropwise to 375 μL of 6.67 M NaOH. The mixture was stirred for 5 h at 60° C. The solution was then cooled to 0° C., carefully acidified to pH 1 with 1 N HCl, and extracted with 5 portions of Et₂O. The combined extracts were washed with a saturated solution of NaCl, dried over Na₂SO₄, and evaporated to dryness. 2-(4-((tert-butoxycarbonyl)amino)butyl)malonic acid was dissolved in 1:1 H₂O:MeCN and lyophilized to give a clear serum. Yield >99%. ¹H NMR (500 MHz, CDCl3) δ 6.29 (s, 1H), 4.57 (s, 1H), 3.35 (t, J=7.0 Hz, 1H), 3.03 (t, J=6.7 Hz, 2H), 1.92-1.85 (m, 2H), 1.43 (p, J=6.7 Hz, 2H), 1.36 (s, 11H). HR-ESI-MS [M−H]−: calculated for C12H20O6N1 m/z 274.1296, found m/z 274.1295.

(S)-6-(Boc-amino)-2-hydroxyhexanoic Acid (24) was synthesized by following a published method⁶⁵.

EXTENDED DATA TABLE 1
Oligonucleotides used in this study

| Name | Sequence (5′-3′) |
|---|---|
| pET32a | ATTTTGTTTAACTTTAAGAAGGAGATATAC |
| MaPylRS | ATATGGGCAGCAGCCATCATCATCATCATC |
| gBlock | ACAGCAGCGGCCTGGTGCCGCGCGGCAGCC |
| | ATATGACGGTGAAATACACTGACGCACAGA |
| | TCCAGCGCCTTCGCGAATATGGGAATGGCA |
| | CGTATGAACAGAAAGTGTTCGAAGATTTGG |
| | CTTCGCGCGACGCAGCCTTTAGCAAAGAAA |
| | TGAGTGTTGCCTCAACCGACAATGAGAAAA |
| | AAATTAAGGGCATGATTGCCAACCCGTCAC |
| | GTCATGGACTTACGCAACTTATGAACGACA |
| | TTGCCGACGCATTAGTCGCTGAGGGATTTA |
| | TCGAGGTCCGCACGCCAATCTTTATCTCAA |
| | AAGACGCGCTTGCCCGTATGACGATTACAG |
| | AAGACAAGCCCCTGTTCAAGCAAGTATTCT |
| | GGATCGACGAGAAGCGTGCCTTACGCCCAA |
| | TGTTGGCTCCAAATTTATATTCCGTTATGC |
| | GTGATTTGCGTGACCACACCGACGGCCCAG |
| | TGAAGATTTTCGAGATGGGGAGCTGTTTTC |
| | GCAAGGAAAGTCACAGTGGCATGCATTTGG |
| | AGGAGTTCACGATGCTGAACCTTGTGGATA |
| | TGGGACCGCGTGGTGATGCGACAGAGGTTT |
| | TAAAAAATTACATTAGTGTTGTGATGAAAG |
| | CAGCGGGATTGCCCGATTATGATTTAGTCC |
| | AGGAAGAGAGTGACGTCTACAAAGAAACTA |
| | TCGATGTTGAGATTAACGGGCAAGAAGTAT |
| | GTAGCGCTGCTGTCGGACCCCATTATCTGG |
| | ATGCTGCCCATGATGTGCATGAACCTTGGT |
| | CTGGTGCTGGTTTCGGTTTGGAGCGCTTAT |
| | TAACCATTCGTGAGAAATATTCCACAGTAA |
| | AGAAAGGGGGGGCAAGTATCTCGTACCTGA |
| | ACGGTGCAAAAATTAATTAACATATGCACC |
| | ATCACCACCATCATTCTTCTGG |
| | (SEQ ID NO: 13) |
| pET32a | ATTTTGTTTAACTTTAAGAAGGAGATATAC |
| MaFRS1 | ATATGGGCAGCAGCCATCATCATCATCATC |
| gBlock | ACAGCAGCGGCCTGGTGCCGCGCGGCAGCC |
| | ATATGACGGTGAAATACACTGACGCACAGA |
| | TCCAGCGCCTTCGCGAATATGGGAATGGCA |
| | CGTATGAACAGAAAGTGTTCGAAGATTTGG |
| | CTTCGCGCGACGCAGCCTTTAGCAAAGAAA |
| | TGAGTGTTGCCTCAACCGACAATGAGAAAA |
| | AAATTAAGGGCATGATTGCCAACCCGTCAC |
| | GTCATGGACTTACGCAACTTATGAACGACA |
| | TTGCCGACGCATTAGTCGCTGAGGGATTTA |
| | TCGAGGTCCGCACGCCAATCTTTATCTCAA |
| | AAGACGCGCTTGCCCGTATGACGATTACAG |
| | AAGACAAGCCCCTGTTCAAGCAAGTATTCT |
| | GGATCGACGAGAAGCGTGCCTTACGCCCAA |
| | TGTTGGCTCCAAATTTATATTCCGTTATGC |
| | GTGATTTGCGTGACCACACCGACGGCCCAG |
| | TGAAGATTTTCGAGATGGGGAGCTGTTTTC |
| | GCAAGGAAAGTCACAGTGGCATGCATTTGG |
| | AGGAGTTCACGATGCTGGCGCTTCTGGATA |
| | TGGGACCGCGTGGTGATGCGACAGAGGTTT |
| | TAAAAAATTACATTAGTGTTGTGATGAAAG |
| | CAGCGGGATTGCCCGATTATGATTTAGTCC |
| | AGGAAGAGAGTGACGTCTACAAAGAAACTA |
| | TCGATGTTGAGATTAACGGGCAAGAAGTAT |
| | GTAGCGCTGCTGTCGGACCCCATTATCTGG |
| | ATGCTGCCCATGATGTGCATGAACCTTGGT |
| | CTGGTGCTGGTTTCGGTTTGGAGCGCTTAT |
| | TAACCATTCGTGAGAAATATTCCACAGTAA |
| | AGAAAGGGGGGGCAAGTATCTCGTACCTGA |
| | ACGGTGCAAAAATTAATTAACATATGCACC |
| | ATCACCACCATCATTCTTCTGG |
| | (SEQ ID NO: 14) |
| MaFRSA | TGTGAGCGGATAACAATTCCCCTCTAGAAA |
| fusion tag | TAATTTTGTTTAACTTTAAGAAGGAGATAT |
| gblock | ACAATGGGCAGCAGCCATCATCATCATCAT |
| | CACAGCAGCGGCCTGGTGCCGCGCGGCAGC |
| | CATATGACGGTGAAATACACTGACGCACAG |
| | ATCCAGCGCCTTCGCGAATATGGGAATGGC |
| | ACGTA |
| | (SEQ ID NO: 15) |
| pMega | CAATTTCACAAAGGAGGTGCGGCCGCATGA |
| MaPylRS | CAGTCAAATATACCGACGCCCAGATTCAGC |
| gBlock | GTTTGCGCGAGTACGGCAACGGTACGTATG |
| | AGCAAAAGGTTTTTGAAGACCTTGCTAGTC |
| | GCGACGCGGCGTTCAGCAAGGAAATGAGTG |
| | TTGCCTCCACAGATAATGAAAAGAAAATCA |
| | AAGGCATGATCGCCAACCCCTCTCGTCACG |
| | GCTTGACCCAGCTGATGAACGATATTGCAG |
| | ATGCGTTGGTAGCTGAGGGGTTCATTGAGG |
| | TGCGTACTCCTATTTTCATTAGTAAGGACG |
| | CCCTTGCACGCATGACTATTACGGAGGATA |
| | AACCTTTGTTCAAACAGGTCTTCTGGATCG |
| | ATGAGAAGCGTGCACTGCGCCCCATGCTGG |
| | CACCCAACTTATATAGTGTCATGCGCGATT |
| | TACGCGATCACACTGACGGTCCGGTGAAAA |
| | TTTTTGAGATGGGCTCATGCTTCCGCAAAG |
| | AGTCGCATTCTGGTATGCACCTGGAAGAGT |
| | TTACCATGTTAAACCTGGTAGACATGGGGC |
| | CACGCGGGGACGCTACGGAGGTCCTGAAGA |
| | ACTACATTAGCGTCGTGATGAAAGCAGCCG |
| | GGTTACCCGATTACGATCTTGTTCAGGAAG |
| | AGAGTGACGTTTACAAAGAAACAATCGATG |
| | TTGAAATCAACGGTCAAGAAGTTTGTTCAG |
| | CGGCCGTGGGGCCGCATTATTTAGACGCTG |
| | CCCACGATGTACATGAGCCGTGGAGTGGTG |
| | CAGGCTTCGGTCTTGAGCGCTTGCTGACCA |
| | TTCGTGAGAAATATAGCACTGTCAAAAAAG |
| | GTGGCGCCTCCATCTCTTATCTTAATGGAG |
| | CTAAAATCAACTAAGCGGCCGCGTTTAAAC |
| | GGTCTCCAGCTTGGCTGTTTTGGCGGATGA |
| | GAGAAGATTTTCAGCCTGATACAGATTAAA |
| | TCAGAACGCAGAAGCGGTCTGATAAAACAG |
| | AATTTGCCTGGCGGCAGTAGCGCGGTGGTC |
| | CCACCTGACCCCATGCCGAACTCAGAAGTG |
| | AAACGCCGTAGCGCCGATGGTAGTGTGGGG |
| | TCTCCCCATGCGAGAGTAGGGAACTGCCAG |
| | GCATCAAATAAAACGAAAGGCTCAGTCGAA |
| | AGACTGGGCCTTGTTTGTGAGCTCCCGGTC |
| | ATCAATCATCCCCATAATCCTTGTTAGCCT |
| | GCAGGTAATTCCGCTTCGCAACATGTGAGC |
| | ACCGGTTTATTGACTACCGGAAGCAGTGTG |
| | ACCGTGTGCTTCTCAAATGCCTGAGGCCAG |
| | TTTGCTCAGGCTCTCCCCGTGGAGGTAATA |
| | ATTGACGATATGATCAGTGCACGGCTAACT |
| | AAGCGGCCTGCTGACTTTCTCGCCGATCAA |
| | AAGGCATTTTGCTATTAAGGGATTGACGAG |
| | GGCGTATCTGCGCAGTAAGATAATTGTGAG |
| | CGGATAACAATTAGCAGACAAGATGGGTCC |
| | CTTATCATGGCAACCATCTGAACGGGGGAC |
| | GGTCCGGCGACCAGCGGGTCTCTAAAACCT |
| | AGCCAGCGGGGTTCGACGCCCCGGTCTCTC |
| | GCCAAATTCGAAAAGCCTGCTCAACGAGCA |
| | GGCTTTTTTGCATGCTCGAGCAGCTCAGGG |
| | TCGAA |
| | (SEQ ID NO: 16) |
| RF31 | GCTGGCGCTTAAGGATATGGGA |
| | (SEQ ID NO: 17) |
| RF32 | ATCGTGAACTCCTCCAAATG |
| | (SEQ ID NO: 18) |
| RF33 | GCTGGCGCTTGCGGATATGGGA |
| | (SEQ ID NO: 19) |
| RF48 | TGGTATGCACCTGGAAGAGTTTA |
| | CCATGTTAGCGCTGGCGGACATG |
| | GGGCCACGCGGGGA |
| | (SEQ ID NO: 20) |
| RF49 | TCCCCGCGTGGCCCCATGTCCGC |
| | CAGCGCTAACATGGTAAACTCTT |
| | CCAGGTGCATACCA |
| | (SEQ ID NO: 21) |
| RF61 | GACATGGGGCCACGC |
| | (SEQ ID NO: 22) |
| RF62 | TAACATGGTAAACTCTTCCAGGTG |
| | CA (SEQ ID NO: 23) |
| CS43 | AGACAACCATTAGCTGTCGACACA |
| | ATC (SEQ ID NO: 24) |
| CS44 | GGTAAAAGGACAGGGCCA |
| | (SEQ ID NO: 25) |
| T7 F | TAATACGACTCACTATAGGG |
| | (SEQ ID NO: 26) |
| T7 R | GCTAGTTATTGCTCAGCGG |
| | (SEQ ID NO: 27) |
| Ma-PylT-F | CTAATACGACTCACTATAGGGGG |
| | ACGGTCCGGCGACCAGCGGGTCT |
| | CTAAAACCTAGCCA |
| | (SEQ ID NO: 28) |
| Ma-PylT-R | TmGGCGAGAGACCGGGGCGTCGA |
| | ACCCCGCTGGCTAGGTTTTAGAG |
| | ACCCGCTGGTCGCCG |
| | (SEQ ID NO: 29) |
| Ma-tRNA^{Pyl} | GGGGGACGGUCCGGCGACCAGCG |
| | GGUCUCUAAAACCUAGCCAGCGG |
| | GGUUCGACGCCCCGGUCUCUCGC |
| | CA (SEQ ID NO: 30) |
| Ma-PylT- | CTAATACGACTCACTATAGGGGG |
| ACC-F | ACGGTCCGGCGACCAGCGGGTCT |
| | ACCAAACCTAGCCA |
| | (SEQ ID NO: 31) |
| Ma-PylT- | TmGGCGAGAGACCGGGGCGTCGA |
| ACC-R | ACCCCGCTGGCTAGGTTTGGTAG |
| | ACCCGCTGGTCGCCG |
| | (SEQ ID NO: 32) |
| Ma- | GGGGGACGGTCCGGCGACCAGCG |
| tRNA^{Pyl} | GGTCTACCAAACCTAGCCAGCGG |
| ACC | GGTTCGACGCCCCGGTCTCTCGC |
| | CA (SEQ ID NO: 33) |
| MGV-flag-1 | GCGAATTAATACGACTCACTATA |
| | GGGTTAACTTTAACAAGGAGAAA |
| | AACATGGGTGTCGACTACAAGGA |
| | CGACGA (SEQ ID NO: 34) |
| MGV-flag-2 | AAACCCCTCCGTTTAGAGAGGGG |
| | TTATGCTAGTTACTTGTCGTCGTC |
| | GTCCTTGTAGTCGACACCCATGT |
| | TTTTC (SEQ ID NO: 35) |

*mG represents 2′-O-methyl-deoxymethylguanosine

Extended Data Table 2. Expected exact masses of acyl-adenosine nucleosides
extracted in LC-HRMS analysis of acyl-tRNA products digested by RNAse A.

| Substrate used | Exact mass |
|---|---|
| N^{ε}-(tert-butoxycarbonyl)-L-lysine (1) | 496.25142 |
| (S)-6-((tert-butoxycarbonyl)amino)-2-hydroxyhexanoic acid (2) | 497.23544 |
| 6-((tert-butoxycarbonyl)amino)hexanoic acid (3) | 481.24052 |
| N^{ε}-(tert-butoxycarbonyl)-D-lysine (D-BocK, 5) | 496.25142 |
| 2-(4-((tert-butoxycarbonyl)amino)butyl)malonic acid (16) | 525.23035 |
| L-phenylalanine (7) | 415.17244 |
| (S)-2-hydroxy-3-phenylpropanoic acid (8) | 416.15646 |
| 3-phenylpropanoic acid (9) | 400.16155 |
| N-methyl-L-phenylalanine (10) | 429.18809 |
| D-phenylalanine (11) | 415.17244 |
| 2-mercapto-3-phenylpropanoic acid (13) | 432.13362 |
| 2-benzylmalonic acid (14) | 444.15137 |
| N-formyl-L-phenylalanine (15) | 443.16736 |
| 2-(3-methylbenzyl)malonic acid (17) | 458.16702 |
| 3-(meta-tolyl)propanoic acid (decarboxylation product of 17) | 414.17720 |
| 2-(3-trifluoromethylbenzyl)malonic acid (18) | 512.13876 |
| 3-(3-(trifluoromethyl)phenyl)propanoic acid (decarboxylation | 468.14893 |
| product of 18) | |
| 2-(3-bromobenzyl)malonic acid (19) | 522.06189 |
| 3-(3-bromophenyl)propanoic acid (decarboxylation product of 19) | 478.07206 |

Extended Data Table 3. Yields of mono- and diacylated
Ma-tRNA^{Pyl} calculated from intact tRNA analysis as shown in FIGS.
S3 to S6 and performed as described in Section IV. The number
on top in each box represents the average of 3 technical replicates,
while the number on the bottom is the standard deviation.

| | Enzyme | | | | | | | |
|---|---|---|---|---|---|---|---|---|
| | MaPylRS | | MaFRS1 | | MaFRS2 | | MaFRSA | |
| Substrate | mono | di | mono | di | mono | di | mono | di |
| 1 | 53.8 | 0.0 | ND | ND | ND | ND | ND | ND |
| | 0.3 | 0.0 | | | | | | |
| 2 | 76.8 | 13.7 | ND | ND | ND | ND | ND | ND |
| | 0.3 | 0.2 | | | | | | |
| 3 | 59.1 | 2.51 | ND | ND | ND | ND | ND | ND |
| | 0.5 | 0.09 | | | | | | |
| 5 | 0 | 0 | ND | ND | ND | ND | ND | ND |
| | 0 | 0 | | | | | | |
| 7 | ND | ND | 36.2 | 29.9 | 47.7 | 17.7 | 0.24 | 0 |
| | | | 1.1 | 0.7 | 0.3 | 0.3 | 0.01 | 0 |
| 8 | ND | ND | 26.4 | 21.0 | 36.5 | 2.78 | ND | ND |
| | | | 1.0 | 0.3 | 0.5 | 0.23 | | |
| 9 | ND | ND | 0.97 | 0 | 1.89 | 0 | ND | ND |
| | | | 0.03 | 0 | 0.02 | 0 | | |
| 10 | ND | ND | 1.73 | 0 | 0.55 | 0 | ND | ND |
| | | | 0.06 | 0 | 0.02 | 0 | | |
| 11 | ND | ND | 0 | 0 | 0 | 0 | ND | ND |
| | | | 0 | 0 | 0 | 0 | | |
| 13 | ND | ND | 0* | 0* | 2.08 | 0 | ND | ND |
| | | | 0 | 0 | 0.06 | 0 | | |
| 14 | ND | ND | 24.4 | 0 | 43.7 | 0 | ND | ND |
| | | | 0.8 | 0 | 0.5 | 0 | | |
| 15 | ND | ND | 9.52 | 40.5 | 29.3 | 1.80 | ND | ND |
| | | | 0.28 | 0.5 | 0.3 | 0.15 | | |
| 16 | 0.25 | 0.0 | ND | ND | ND | ND | ND | ND |
| | 0.02 | 0.0 | | | | | | |
| 17 | ND | ND | 49.8 | 3.64 | 52.0 | 0 | 9.48 | 1.54 |
| | | | 0.7 | 0.35 | 0.1 | 0 | 0.07 | 0.10 |
| 18 | ND | ND | 59.3 | 9.24 | 26.1 | 0 | 31.4 | 0 |
| | | | 1.0 | 0.60 | 0.4 | 0 | 0.7 | 0 |
| 19 | ND | ND | 37.4 | 10.8 | 32.7 | 0 | 22.3 | 0 |
| | | | 0.7 | 1.4 | 0.7 | 0 | 0.4 | 0 |

*No product was observed when 13 was incubated with 2.5 μM MaFRS1 and 25 μM Ma-tRNA^{Pyl}. Increasing [MaFRS1] to 12.5 μM led to 0.72 ± 0.02% and 9.72 ± 0.31% of mono- and diacylated tRNA, respectively.
ND = not determined.

Extended Data Table 4. Structure refinement statistics.
MaFRSA, meta-CF₃-2-BMA, AMP-PNP

| Wavelength (Å) | 1.11583 | |
|---|---|---|
| Resolution range (Å) | 45.37-1.803 | (1.867-1.803) |
| Space group | I 4 | |
| Unit cell dimensions (Å) | 108.958 108.958 112.26 90° 90° 90° | |
| Total reflections | 115764 | (9210) |
| Unique reflections | 57972 | (4659) |
| Multiplicity | 2.0 | (2.0) |
| Completeness (%) | 95.02 | (66.69) |
| Mean I/sigma (I) | 18.75 | (0.43) |
| Wilson B-factor | 39.02 | |
| R-merge | 0.02241 | (1.976) |
| R-meas | 0.03169 | (2.794) |
| R-pim | 0.02241 | (1.976) |
| CC1/2 | 1 | (0.106) |
| CC* | 1 | (0.437) |
| Reflections used in refinement | 57275 | (4008) |
| Reflections used for R-free | 2924 | (215) |
| R-work | 0.1800 | (0.4105) |
| R-free | 0.2115 | (0.4402) |
| CC(work) | 0.969 | (0.410) |
| CC(free) | 0.955 | (0.270) |

| | |
|---|---|
| Number of non-hydrogen atoms | 4931 |
| macromolecules | 4367 |
| ligands | 111 |
| solvent | 453 |
| Protein residues | 553 |
| RMS(bonds) (Å) | 0.011 |
| RMS(angles) (°) | 1.1 |
| Ramachandran favored (%) | 98 |
| Ramachandran allowed (%) | 2 |
| Ramachandran outliers (%) | 0 |
| Rotamer outliers (%) | 0.64 |
| Clashscore | 5.2 |
| Average B-factor | 47.8 |
| macromolecules | 47.42 |
| ligands | 55.19 |
| solvent | 49.59 |

*Parentheses indicate values for last resolution shell

Extended Data Table 5. LC-MS/MS analysis of sfGFP samples generated
in DH10B and DH10B ΔaspC ΔtyrB. Sample numbers refer
to column values shown in FIG. 6E. All values are in %.

| | Sample | | | | | | | | |
|---|---|---|---|---|---|---|---|---|---|
| | i | ii | iii | iv | v | vi | vii | viii | ix |
| vari- | substrate | | | | | | | | |
| ant | WT | 1 | 2 | 20 | 21 | 20 | 21 | 20 | 21 |
| Y | 100.00 | 0.36 | 0.05 | 0.28 | 0.59 | 0.15 | 0.95 | 0.60 | 1.57 |
| 1 | 0.00 | 94.56 | 0.07 | 0.00 | 0.00 | 0.00 | 0.00 | 0.00 | 0.00 |
| 2 | 0.00 | 4.44 | 99.82 | 0.00 | 0.00 | 0.00 | 0.00 | 0.00 | 0.00 |
| 20 | 0.00 | 0.00 | 0.00 | 98.73 | 85.35 | 97.14 | 51.54 | 98.19 | 52.31 |
| 21 | 0.00 | 0.00 | 0.00 | 0.32 | 11.23 | 0.63 | 38.48 | 0.65 | 44.10 |
| A | 0.00 | 0.02 | 0.00 | 0.03 | 0.17 | 0.14 | 0.76 | 0.02 | 0.00 |
| F | 0.00 | 0.14 | 0.01 | 0.34 | 0.61 | 0.15 | 0.20 | 0.34 | 0.27 |
| K | 0.00 | 0.32 | 0.01 | 0.02 | 0.13 | 0.14 | 0.90 | 0.01 | 0.53 |
| Q | 0.00 | 0.08 | 0.03 | 0.11 | 0.81 | 0.62 | 3.49 | 0.07 | 0.07 |
| W | 0.00 | 0.08 | 0.00 | 0.17 | 1.11 | 1.02 | 3.68 | 0.12 | 0.48 |

Extended Data Data 1. Masses Identified in Deconvoluted Mass Spectra and their Identities

N-1 and N-2 refer to the tRNA missing the final 1 and 2 nucleotides at the 3′ end, respectively. -P and -PPP refer to whether the 5′ end of the tRNA has a monophosphate or a triphosphate. N+G and N+GG refer to tRNA products with non-templated addition of guanosine residues identified in the mass spectrum. Note that for some enzyme/substrate pairs there is evidence that N+G products are acylated by the synthetase, indicating that the untemplated guanosine addition does not exclude these tRNA species from activity with the synthetase.

1. A method to generate novel acyl-tRNA species, comprising reacting an orthogonal synthetase with a tRNA and a non-L-α-amino acid selected from α-hydroxy acids, α-thio acids, N-formyl-L-α-amino acids and α-carboxyl acid monomers (malonic acids) that are formally precursors to polyketide natural products, to generate the novel acyl-tRNA species.

2. The method of claim 1, wherein the orthogonal synthetase accepts α-hydroxy acids, α-thio acids, N-formyl-L-α-amino acids, and α-carboxyl acid monomers (malonic acids) that are formally precursors to polyketide natural products.

3. The method of claim 1, wherein the orthogonal synthetase is a pyrrolysyl-tRNA synthetase (PylRS).

4. The method of claim 1, wherein the orthogonal synthetase is a pyrrolysyl-tRNA synthetase (PylRS), and the PylRS is a Methanomethylophilus alvus PylRS (MaPylRS) or a MaPylRS substitution variant.

5. The method of claim 1, wherein the orthogonal synthetase is a pyrrolysyl-tRNA synthetase (PylRS), and the PylRS is a Methanomethylophilus alvus PylRS (MaPylRS) substitution variant comprising substitutions at N166 and V168.

6. The method of claim 1, wherein the orthogonal synthetase is a pyrrolysyl-tRNA synthetase (PylRS), and the PylRS is a Methanomethylophilus alvus PylRS (MaPylRS) substitution variant comprising MaFRS1 (N166A, V168L), MaFRS2 (N166A, V168K), or MaFRSA (N166A, V168A).

7. The method of claim 1, further comprising providing the acyl-tRNA species in a translation system, wherein the non-L-α-amino acid is incorporated into a protein.

8. The method of claim 1, further comprising providing the acyl-tRNA species in a translation system, wherein the non-L-α-amino acid is incorporated into a sequence-defined non-protein hetero-polymer.

9. A composition or kit comprising an isolated orthogonal synthetase that accepts α-hydroxy acids, α-thio acids, N-formyl-L-α-amino acids or α-carboxyl acid monomers (malonic acids) that are formally precursors to polyketide natural products.

10. The composition or kit of claim 9, wherein the orthogonal synthetase accepts α-hydroxy acids, α-thio acids, N-formyl-L-α-amino acids, and α-carboxyl acid monomers (malonic acids) that are formally precursors to polyketide natural products.

11. The composition or kit of claim 10, wherein the orthogonal synthetase is a pyrrolysyl-tRNA synthetase (PylRS).

12. The composition or kit of claim 11, wherein the PylRS is a Methanomethylophilus alvus PylRS (MaPylRS) or a MaPylRS variant.

13. The composition or kit of claim 12, wherein the PylRS is MaPylRS variant comprising substitutions at N166 and V168.

14. The composition or kit of claim 13, wherein the PylRS is MaPylRS variant MaFRS1 (N166A, V168L), MaFRS2 (N166A, V168K), or MaFRSA (N166A, V168A).


SEQUENCE LISTING (35 sequences)

SEQ ID NO: 1 - protein, 9 aa, Homo sapiens
VDYKDDDDK

SEQ ID NO: 2 - protein, 275 aa, Methanomethylophilus alvus
MTVKYTDAQIQRLREYGNGTYEQKVFEDLASRDAAFSKEMSVASTDNEKKIKGMIANPSRHGLTQLMNDIADALVAEGFIEVRTPIFISKDALARMTITEDKPLFKQVFWIDEKRALRPMLAPNLYSVMRDLRDHTDGPVKIFEMGSCFRKESHSGMHLEEFTMLNLVDMGPRGDATEVLKNYISVVMKAAGLPDYDLVQEESDVYKETIDVEINGQEVCSAAVGPHYLDAAHDVHEPWSGAGFGLERLLTIREKYSTVKKGGASISYLNGAKIN

SEQ ID NO: 3 - protein, 270 aa, Methanosarcina mazei
ASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMLAPNLYNYLRKLDRALPDPIKIFEIGPCYRKESDGKEHLEEFTMLNFCQMGSGCTRENLESIITDFLNHLGIDFKIVGDSCMVYGDTLDVMHGDLELSSAVVGPIPLDREWGIDKPWIGAGFGLERLLKVKHDFKNIKRAARSESYYNGISTNL

SEQ ID NO: 4 - protein, 275 aa, Methanomethylophilus alvus
MTVKYTDAQIQRLREYGNGTYEQKVFEDLASRDAAFSKEMSVASTDNEKKIKGMIANPSRHGLTQLMNDIADALVAEGFIEVRTPIFISKDALARMTITEDKPLFKQVFWIDEKRALRPMLAPNLYSVMRDLRDHTDGPVKIFEMGSCFRKESHSGMHLEEFTMLALLDMGPRGDATEVLKNYISVVMKAAGLPDYDLVQEESDVYKETIDVEINGQEVCSAAVGPHYLDAAHDVHEPWSGAGFGLERLLTIREKYSTVKKGGASISYLNGAKIN

SEQ ID NO: 5 - protein, 275 aa, Methanomethylophilus alvus
MTVKYTDAQIQRLREYGNGTYEQKVFEDLASRDAAFSKEMSVASTDNEKKIKGMIANPSRHGLTQLMNDIADALVAEGFIEVRTPIFISKDALARMTITEDKPLFKQVFWIDEKRALRPMLAPNLYSVMRDLRDHTDGPVKIFEMGSCFRKESHSGMHLEEFTMLALKDMGPRGDATEVLKNYISVVMKAAGLPDYDLVQEESDVYKETIDVEINGQEVCSAAVGPHYLDAAHDVHEPWSGAGFGLERLLTIREKYSTVKKGGASISYLNGAKIN

SEQ ID NO: 6 - protein, 275 aa, Methanomethylophilus alvus
MTVKYTDAQIQRLREYGNGTYEQKVFEDLASRDAAFSKEMSVASTDNEKKIKGMIANPSRHGLTQLMNDIADALVAEGFIEVRTPIFISKDALARMTITEDKPLFKQVFWIDEKRALRPMLAPNLYSVMRDLRDHTDGPVKIFEMGSCFRKESHSGMHLEEFTMLALADMGPRGDATEVLKNYISVVMKAAGLPDYDLVQEESDVYKETIDVEINGQEVCSAAVGPHYLDAAHDVHEPWSGAGFGLERLLTIREKYSTVKKGGASISYLNGAKIN

SEQ ID NO: 7 - protein, 19 aa, Homo sapiens
GSSHHHHHHSSGLVPRGSH

SEQ ID NO: 8 - protein, 246 aa, Aequorea victoria
MSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGSHHHHHH

SEQ ID NO: 9 - protein, 245 aa, Aequorea victoria
MSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHLSTQSVLSKDPNEKRDHMVLLEFVTAAGITHGMDELYKGSHHHHHH

SEQ ID NO: 10 - protein, 19 aa, Homo sapiens
NHYLSTQSVLSKDPNEKRD

SEQ ID NO: 11 - protein, 17 aa, Homo sapiens
YLSTQSVLSKDPNEKRD

SEQ ID NO: 12 - protein, 11 aa, Homo sapiens
MGVDYKDDDDK

SEQ ID NO: 13 - DNA, 952 bp, Synthetic construct
  source 1..952: mol_type = other DNA
attttgtttaactttaagaaggagatatacatatgggcagcagccatcatcatcatcatcacagcagcggcctggtgccgcgcggcagccatatgacggtgaaatacactgacgcacagatccagcgccttcgcgaatatgggaatggcacgtatgaacagaaagtgttcgaagatttggcttcgcgcgacgcagcctttagcaaagaaatgagtgttgcctcaaccgacaatgagaaaaaaattaagggcatgattgccaacccgtcacgtcatggacttacgcaacttatgaacgacattgccgacgcattagtcgctgagggatttatcgaggtccgcacgccaatctttatctcaaaagacgcgcttgcccgtatgacgattacagaagacaagcccctgttcaagcaagtattctggatcgacgagaagcgtgccttacgcccaatgttggctccaaatttatattccgttatgcgtgatttgcgtgaccacaccgacggcccagtgaagattttcgagatggggagctgttttcgcaaggaaagtcacagtggcatgcatttggaggagttcacgatgctgaaccttgtggatatgggaccgcgtggtgatgcgacagaggttttaaaaaattacattagtgttgtgatgaaagcagcgggattgcccgattatgatttagtccaggaagagagtgacgtctacaaagaaactatcgatgttgagattaacgggcaagaagtatgtagcgctgctgtcggaccccattatctggatgctgcccatgatgtgcatgaaccttggtctggtgctggtttcggtttggagcgcttattaaccattcgtgagaaatattccacagtaaagaaagggggggcaagtatctcgtacctgaacggtgcaaaaattaattaacatatgcaccatcaccaccatcattcttctgg

SEQ ID NO: 14 - DNA, 952 bp, Synthetic construct
  source 1..952: mol_type = other DNA
attttgtttaactttaagaaggagatatacatatgggcagcagccatcatcatcatcatcacagcagcggcctggtgccgcgcggcagccatatgacggtgaaatacactgacgcacagatccagcgccttcgcgaatatgggaatggcacgtatgaacagaaagtgttcgaagatttggcttcgcgcgacgcagcctttagcaaagaaatgagtgttgcctcaaccgacaatgagaaaaaaattaagggcatgattgccaacccgtcacgtcatggacttacgcaacttatgaacgacattgccgacgcattagtcgctgagggatttatcgaggtccgcacgccaatctttatctcaaaagacgcgcttgcccgtatgacgattacagaagacaagcccctgttcaagcaagtattctggatcgacgagaagcgtgccttacgcccaatgttggctccaaatttatattccgttatgcgtgatttgcgtgaccacaccgacggcccagtgaagattttcgagatggggagctgttttcgcaaggaaagtcacagtggcatgcatttggaggagttcacgatgctggcgcttctggatatgggaccgcgtggtgatgcgacagaggttttaaaaaattacattagtgttgtgatgaaagcagcgggattgcccgattatgatttagtccaggaagagagtgacgtctacaaagaaactatcgatgttgagattaacgggcaagaagtatgtagcgctgctgtcggaccccattatctggatgctgcccatgatgtgcatgaaccttggtctggtgctggtttcggtttggagcgcttattaaccattcgtgagaaatattccacagtaaagaaagggggggcaagtatctcgtacctgaacggtgcaaaaattaattaacatatgcaccatcaccaccatcattcttctgg

SEQ ID NO: 15 - DNA, 185 bp, Methanomethylophilus alvus
  source 1..185: mol_type = genomic DNA
tgtgagcggataacaattcccctctagaaataattttgtttaactttaagaaggagatatacaatgggcagcagccatcatcatcatcatcacagcagcggcctggtgccgcgcggcagccatatgacggtgaaatacactgacgcacagatccagcgccttcgcgaatatgggaatggcacgta

SEQ ID NO: 16 - DNA, 1595 bp, Synthetic construct
  source 1..1595: mol_type = other DNA
caatttcacaaaggaggtgcggccgcatgacagtcaaatataccgacgcccagattcagcgtttgcgcgagtacggcaacggtacgtatgagcaaaaggtttttgaagaccttgctagtcgcgacgcggcgttcagcaaggaaatgagtgttgcctccacagataatgaaaagaaaatcaaaggcatgatcgccaacccctctcgtcacggcttgacccagctgatgaacgatattgcagatgcgttggtagctgaggggttcattgaggtgcgtactcctattttcattagtaaggacgcccttgcacgcatgactattacggaggataaacctttgttcaaacaggtcttctggatcgatgagaagcgtgcactgcgccccatgctggcacccaacttatatagtgtcatgcgcgatttacgcgatcacactgacggtccggtgaaaatttttgagatgggctcatgcttccgcaaagagtcgcattctggtatgcacctggaagagtttaccatgttaaacctggtagacatggggccacgcggggacgctacggaggtcctgaagaactacattagcgtcgtgatgaaagcagccgggttacccgattacgatcttgttcaggaagagagtgacgtttacaaagaaacaatcgatgttgaaatcaacggtcaagaagtttgttcagcggccgtggggccgcattatttagacgctgcccacgatgtacatgagccgtggagtggtgcaggcttcggtcttgagcgcttgctgaccattcgtgagaaatatagcactgtcaaaaaaggtggcgcctccatctcttatcttaatggagctaaaatcaactaagcggccgcgtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggccttgtttgtgagctcccggtcatcaatcatccccataatccttgttagcctgcaggtaattccgcttcgcaacatgtgagcaccggtttattgactaccggaagcagtgtgaccgtgtgcttctcaaatgcctgaggccagtttgctcaggctctccccgtggaggtaataattgacgatatgatcagtgcacggctaactaagcggcctgctgactttctcgccgatcaaaaggcattttgctattaagggattgacgagggcgtatctgcgcagtaagataattgtgagcggataacaattagcagacaagatgggtcccttatcatggcaaccatctgaacgggggacggtccggcgaccagcgggtctctaaaacctagccagcggggttcgacgccccggtctctcgccaaattcgaaaagcctgctcaacgagcaggcttttttgcatgctcgagcagctcagggtcgaa

SEQ ID NO: 17 - DNA, 22 bp, Synthetic construct
  source 1..22: mol_type = other DNA
gctggcgcttaaggatatggga

SEQ ID NO: 18 - DNA, 20 bp, Synthetic construct
  source 1..20: mol_type = other DNA
atcgtgaactcctccaaatg

SEQ ID NO: 19 - DNA, 22 bp, Synthetic construct
  source 1..22: mol_type = other DNA
gctggcgcttgcggatatggga

SEQ ID NO: 20 - DNA, 60 bp, Synthetic construct
  source 1..60: mol_type = other DNA
tggtatgcacctggaagagtttaccatgttagcgctggcggacatggggccacgcgggga

SEQ ID NO: 21 - DNA, 60 bp, Synthetic construct
  source 1..60: mol_type = other DNA
tccccgcgtggccccatgtccgccagcgctaacatggtaaactcttccaggtgcatacca

SEQ ID NO: 22 - DNA, 15 bp, Synthetic construct
  source 1..15: mol_type = other DNA
gacatggggccacgc

SEQ ID NO: 23 - DNA, 26 bp, Synthetic construct
  source 1..26: mol_type = other DNA
taacatggtaaactcttccaggtgca

SEQ ID NO: 24 - DNA, 27 bp, Synthetic construct
  source 1..27: mol_type = other DNA
agacaaccattagctgtcgacacaatc

SEQ ID NO: 25 - DNA, 18 bp, Synthetic construct
  source 1..18: mol_type = other DNA
ggtaaaaggacagggcca

SEQ ID NO: 26 - DNA, 20 bp, Synthetic construct
  source 1..20: mol_type = other DNA
taatacgactcactataggg

SEQ ID NO: 27 - DNA, 19 bp, Synthetic construct
  source 1..19: mol_type = other DNA
gctagttattgctcagcgg

SEQ ID NO: 28 - DNA, 60 bp, Methanomethylophilus alvus
  source 1..60: mol_type = other DNA
ctaatacgactcactatagggggacggtccggcgaccagcgggtctctaaaacctagcca

SEQ ID NO: 29 - DNA, 60 bp, Methanomethylophilus alvus
  source 1..60: mol_type = other DNA
  modified_base 2: mod_base = OTHER; note = 2'-O-methyl-deoxymethylguanosine
tggcgagagaccggggcgtcgaaccccgctggctaggttttagagacccgctggtcgccg

SEQ ID NO: 30 - RNA, 71 nt, Methanomethylophilus alvus
  source 1..71: mol_type = tRNA
  modified_base 10: mod_base = OTHER; note = uracil
  modified_base 26: mod_base = OTHER; note = uracil
  modified_base 28: mod_base = OTHER; note = uracil
  modified_base 30: mod_base = OTHER; note = uracil
  modified_base 37: mod_base = OTHER; note = uracil
  modified_base 49: mod_base = OTHER; note = uracil
  modified_base 50: mod_base = OTHER; note = uracil
  modified_base 62: mod_base = OTHER; note = uracil
  modified_base 64: mod_base = OTHER; note = uracil
  modified_base 66: mod_base = OTHER; note = uracil
gggggacggtccggcgaccagcgggtctctaaaacctagccagcggggttcgacgccccggtctctcgcca

SEQ ID NO: 31 - DNA, 60 bp, Methanomethylophilus alvus
  source 1..60: mol_type = other DNA
ctaatacgactcactatagggggacggtccggcgaccagcgggtctaccaaacctagcca

SEQ ID NO: 32 - DNA, 60 bp, Methanomethylophilus alvus
  source 1..60: mol_type = other DNA
  modified_base 2: mod_base = OTHER; note = 2'-O-methyl-deoxymethylguanosine
tggcgagagaccggggcgtcgaaccccgctggctaggtttggtagacccgctggtcgccg

SEQ ID NO: 33 - DNA, 71 bp, Methanomethylophilus alvus
  source 1..71: mol_type = other DNA
gggggacggtccggcgaccagcgggtctaccaaacctagccagcggggttcgacgccccggtctctcgcca

SEQ ID NO: 34 - DNA, 75 bp, Synthetic construct
  source 1..75: mol_type = other DNA
gcgaattaatacgactcactatagggttaactttaacaaggagaaaaacatgggtgtcgactacaaggacgacga

SEQ ID NO: 35 - DNA, 75 bp, Synthetic construct
  source 1..75: mol_type = other DNA
aaacccctccgtttagagaggggttatgctagttacttgtcgtcgtcgtccttgtagtcgacacccatgtttttc